(19) United States
(12) Patent Application Publication
(10) Pub. No.: US 2018/0185415 A1
(43) Pub. Date: Jul. 5, 2018

(54) RETROVIRAL VECTORS CONTAINING A REVERSE ORIENTATION HUMAN UBIQUITIN C PROMOTER

(71) Applicant: The Regents of the University of California, Oakland, CA (US)

(72) Inventors: Donald B. Kohn, Tarzana, CA (US); Aaron Ross Cooper, Los Angeles, CA (US)

(21) Appl. No.: 15/738,545

(22) PCT Filed: Jun. 22, 2016
(86) PCT No.: PCT/US2016/038814

§ 371 Date: Dec. 20, 2017

Related U.S. Application Data
(60) Provisional application No. 62/187,678, filed on Jul. 1, 2015.

(51) Int. Cl. A61K 35/28; C12N 15/86; C12N 5/0789

(52) U.S. Cl. CPC A61K 35/28; C12N 15/86; C12N 2510/00; C12N 2740/16043; C12N 2830/85; C12N 5/0647

(57) ABSTRACT

In certain embodiments a recombinant retroviral vector is provided where the vector comprises a human ubiquitin C (UBC) promoter operably linked to a transgene where the promoter and the transgene are in a reverse orientation so that the direction of transcription of the transgene from the promoter is oriented towards a 5′ long terminal repeat (LTR) of the vector.

### CROSS-REFERENCE TO RELATED APPLICATIONS

This application claims priority to and benefit of U.S. Ser. No. 62/187,678, filed Jul. 1, 2015, which is incorporated herein by reference in its entirety for all purposes.

### STATEMENT OF GOVERNMENTAL SUPPORT

This invention was made with government support under Grant No. HL073104 awarded by the National Heart Lung and Blood Institute at the National Institutes of Health. The Government has certain rights in this invention.

### BACKGROUND

Gene delivery into human cells has been explored as a means to correct or protect against genetic alterations in a variety of human diseases such as congenital enzyme deficiencies or hematological malignancies. Various gene transduction systems have been developed, including oncoretroviral vectors, lentiviral vectors, adenoviral vectors and adeno associated viral vectors. However, despite the variety of vector systems, cell transduction efficiency can still be too low for therapeutic efficacy.

In spite of the well-recognized need for effective vectors, high-level expression of transgenes in the majority of target cells has been a significant challenge for gene transfer technology.

### SUMMARY

In various embodiments retroviral vectors comparing a human ubiquitin C promoter in a reverse orientation are provided as well as viral particles containing such vectors, host cells transduced with such vectors, and methods of treatment utilizing such

Various embodiments contemplated herein may include, but need not be limited to, one or more of the following:

### Embodiment 1

A recombinant retroviral vector, said vector including a human ubiquitin C (UBC) promoter and a multiple cloning site, wherein said UBC promoter is in a reverse orientation in said vector so that the direction of transcription from said promoter is oriented towards a 5′ long terminal repeat (LTR) of said vector and transcribes a nucleic acid inserted in said multiple cloning site.

### Embodiment 2

A recombinant retroviral vector, said vector including a human ubiquitin C (UBC) promoter operably linked to a transgene wherein said promoter and said transgene are in a reverse orientation so that the direction of transcription of said transgene from said promoter is oriented towards a 5′ long terminal repeat (LTR) of said vector.

### Embodiment 3

The vector according to any one of embodiments 1-2, wherein said promoter includes or consists of a fragment from the human ubiquitin C gene UCSC human genome sequence version hg19 minus strand from about position 125398318 to about position 125399530.

### Embodiment 4

The vector according to any one of embodiments 1-3, wherein an intron within said promoter is not lost during retroviral packaging.

### Embodiment 5

The vector according to any one of embodiments 1-4, wherein said vector contains a polyadenylation signal in reverse orientation.

### Embodiment 6

The vector of embodiment 5, wherein said polyadenylation signal (polyA) is inserted 3′ of said promoter which is 5′ of said promoter with respect to the entire vector sequence.

### Embodiment 7

The vector according to any one of embodiments 5-6, wherein said polyadenylation signal is selected from the group consisting of a bovine growth hormone polyadenylation signal sequence, human growth hormone polyadenylation signal, a rabbit β-globin gene polyadenylation signal, a human herpes virus (HSV) polyadenylation signal, a thymidine kinase (TK) gene polyadenylation signal, and other signals derived from existing genomes or designed in silico and synthesized.

### Embodiment 8

The vector according to any one of embodiments 5-6, wherein said polyadenylation signal is a bovine growth hormone polyadenylation signal sequence or a human growth hormone polyadenylation signal.

### Embodiment 9

The vector according to any one of embodiments 1-8, wherein said vector provides at least about a 2-fold increase in expression in transient transfected and stable-transduced cell lines as compared to the same vector with a UBC promoter in a non-reversed orientation.

### Embodiment 10

The vector according to any one of embodiments 1-9, wherein said vector provides at least about a 4-fold increase in expression in transduced primary cells as compared to the same vector with a UBC promoter in a non-reversed orientation.

### Embodiment 11

The vector according to any one of embodiments 1-10, wherein said retroviral vector is selected from group consisting of an HIV-1 lentiviral vector, an HIV-2 lentiviral vector, an alpharetroviral vector, an equine infectious anemia virus (EIAV) lentiviral vector, an MoMLV vector, an X-MLV vector, a P-MLV vector, a A-MLV vector, a GALV vector, an HEV-W vector, an SIV-1 vector, an FIV-1 vector, and an SERV-1-5 vector.

### Embodiment 12

The vector of embodiment 11, wherein said retroviral vector is a lentiviral vector.

### Embodiment 13

The vector of embodiment 12, wherein said retroviral vector is an HIV-1 based lentiviral vector.

### Embodiment 14

The vector according to any one of embodiments 12-13, wherein said lentiviral vector is a TAT-independent and self-inactivating (SIN) lentiviral vector

### Embodiment 15

The vector according to any one of embodiments 1-14, wherein said vector is a bidirectional vector.

### Embodiment 16

The vector according to any one of embodiments 1-15, further including an insulator in the 3′ LTR.

### Embodiment 17

The vector of embodiment 16, wherein said insulator includes FB (FII/BEAD-A), a 77 bp insulator element, which contains the minimal CTCF binding site enhancer-blocking components of the chicken β-globin 5′ DnaseI-hypersensitive site 4 (5′ HS4).

### Embodiment 18

The vector according to any one of embodiments 1-17, wherein said vector includes a ψ region vector genome packaging signal.

### Embodiment 19

The vector according to any one of embodiments 1-18, wherein said vector includes a Rev Responsive Element (RRE).

### Embodiment 20

The vector according to any one of embodiments 1-19, wherein said vector includes a central polypurine tract.

### Embodiment 21

The vector according to any one of embodiments 1-20, wherein said vector includes a post-translational regulatory element.

### Embodiment 22

The vector of embodiment 21, wherein the posttranscriptional regulatory element is modified Woodchuck Post-transcriptional Regulatory Element (WPRE).

### Embodiment 23

The vector according to any one of embodiments 1-22, wherein said vector is incapable of reconstituting a wild-type lentivirus through recombination.

### Embodiment 24

The vector according to any one of embodiments 2-23, wherein said vector includes a transgene operably linked to said UBC promoter wherein said transgene expresses a gene product for the treatment of a pathology selected from the group consisting of SCID, sickle cell disease, a liposomal storage disease, cystic fibrosis, muscular dystrophy, phenylketonuria, Parkinson's disease, and haemophilia.

### Embodiment 25

The vector according to any one of embodiments 2-15, wherein said vector expresses one or more gene products selected from the group consisting of adenosine deaminase (ADA), IL-2 receptor gamma (IL-2Rγ), purine nucleoside phosphorylase (PNP) gene, Janus kinase-3 (JAK3), Artemis gene, anti-sickling human β-globin gene, Factor VIII, Factor IX, CFTR, full length or shortened dystrophin, ABCD1 gene, TH, AADC, and GCH1, Aspartylglucosaminidase, α-Galactosidase A, Palmitoyl Protein Thioesterase, Tripeptidyl Peptidase, Lysosomal Transmembrane Protein, Cysteine transporter, Acid ceramidase, Acid α-L-fucosidase, Protective protein/cathepsin A, Acid β-glucosidase, Acid β-galactosidase, Iduronate-2-sulfatase, α-L-Iduronidase, Galactocerebrosidase, Acid α-mannosidase, Acid β-mannosidase, Arylsulfatase B, Arylsulfatase A, N-Acetylgalactosamine-6-sulfate, Acid β-galactosidase, N-Acety lglucosamine-1-phosphotransferase, Acid sphingomyelinase (aSM), NPC-1, α-glucosidase, β-Hexosaminidase B, Heparan N-sulfatase, α-N-Acetylglucosaminidase, Acetyl-CoA: α-glucosaminide, N-Acetylglucosamine-6-sulfate, α-N-Acetylgalactosaminidase, α-N-Acetylgalactosaminidase, α-Neuramidase, β-Glucuronidase, β-Hexosaminidase A, Acid Lipase

### Embodiment 26

The vector of embodiment 24, wherein said transgene expresses adenosine deaminase (ADA) for the treatment of ADA-SCID.

### Embodiment 27

The vector of embodiment 24, wherein said transgene expresses IL-2 receptor gamma (IL-2Rγ) gene/cDNA for the treatment of X-SCID.

### Embodiment 28

The vector of embodiment 24, wherein said transgene expresses an anti-sickling human β-globin gene.

### Embodiment 29

The vector of embodiment 28, wherein said anti-sickling human β-globin gene includes about 2.3 kb of recombinant human β-globin gene including exons and introns under the control of the human β-globin gene 5′ promoter and the human β-globin 3′ enhancer.

### Embodiment 30

The vector embodiment 29, wherein said β-globin gene includes β-globin intron 2 with a 375 bp RsaI deletion from IVS2, and a composite human β-globin locus control region including HS2, HS3, and HS4.

### Embodiment 31

A viral particle including a vector according to any one of embodiments 1-23.

### Embodiment 32

A host cell transduced with a vector according to any one of embodiments 2-23.

### Embodiment 33

The host cell of embodiment 32, wherein the cell is a stem cell.

### Embodiment 34

The host cell of embodiment 33, wherein said cell is a stem cell derived from bone marrow.

### Embodiment 35

The host cell of embodiment 33, wherein said cell is a stem cell that is not derived from an embryo or embryonic tissue.

### Embodiment 36

The host cell of embodiment 32, wherein the cell is a 293T cell.

### Embodiment 37

The host cell of embodiment 32, wherein, wherein the cell is a human hematopoietic progenitor cell.

### Embodiment 38

The host cell of embodiment 37, wherein the human hematopoietic progenitor cell is a CD34⁺ cell.

### Embodiment 39

The host cell of embodiment 37, wherein the human hematopoietic progenitor cell is a CD34⁺/CD38⁻ cell.

### Embodiment 40

A composition for the treatment of a pathology shown in column A below, including a pharmaceutically acceptable carrier and a stem cell and/or progenitor cell transfected with a vector according to any one of embodiments 2-23, wherein said vector contains one or more transgenes for the treatment of said pathology as shown in column B below:

| A | B |
|---|---|
| Pathology | Transgene/gene product |
| ADA-SCID | adenosine deaminase (ADA) |
| X-SCID | IL-2 receptor gamma (IL-2Rγ) |
| PNP-SCID | PNP gene |
| JAK3 | Janus kinase-3 (JAK3) |
| Artemis/DCLRE1C | Artemis gene |
| Sickle Cell Disease | anti-sickling human β-globin gene |
| Haemophilia A | Factor VIII |
| Haemophilia B | Factor IX |
| Cystic fibrosis | CFTR |
| Muscular Dystrophy | full length or shortened dystrophin |
| Adrenoleukodystrophy (ALD) | ABCD1 gene |
| Parkinson's Disease | TH, AADC, and GCH1 |
| Phenylketonuria | phenylalanine hydroxylase (PAH) |
| Aspartylglucosaminuria | Aspartylglucosaminidase |
| Fabry | α-Galactosidase A |
| Infantile Batten Disease | Palmitoyl Protein Thioesterase |
| Classic Late Infantile Batten Disease | Tripeptidyl Peptidase |
| Juvenile Batten Disease (CNL2) | Lysosomal Transmembrane Protein |
| Cystinosis | Cysteine transporter |
| Farber | Acid ceramidase |
| Fucosidosis | Acid α-L-fucosidase |
| Galactosidosialidosis | Protective protein/cathepsin A |
| Gaucher types 1, 2, and 3 | Acid β-glucosidase |
| GMl gangliosidosis | Acid β-galactosidase |
| Hunter | Iduronate-2-sulfatase |
| Hurler-Scheie | α-L-Iduronidase |
| Krabbe | Galactocerebrosidase. |
| α-Mannosidosis | Acid α-mannosidase. |
| β-Mannosidosis | Acid β-mannosidase |
| Maroteaux-Lamy | Arylsulfatase B |
| Metachromatic leukodystrophy | Arylsulfatase A |
| Morquio A | N-Acetylgalactosamine-6-sulfate |
| Morquio B | Acid β-galactosidase |
| Mucolipidosis II/III | N-Acety lglucosamine-1 -phospho- |
| | transferase |
| Niemann-PickA, B | Acid sphingomyelinase (aSM) |
| Niemann-Pick C | NPC-1 |
| Pompe Acid | α-glucosidase |
| Sandhoff | β-Hexosaminidase B |
| Sanfilippo A | Heparan N-sulfatase |
| Sanfilippo B | α-N-Acetylglucosaminidase |
| Sanfilippo C | Acetyl-CoA: α-glucosaminide |
| Sanfilippo D | N-Acetylglucosamine-6-sulfate |
| Schindler Disease | α-N-Acetylgalactosaminidase |
| Schindler-Kanzaki. | α-N-Acetylgalactosaminidase |
| Sialidosis | α-Neuramidase |
| Sly | β-Glucuronidase |
| Tay-Sachs | β-Hexosaminidase A |
| Wolman | Acid Lipase. |

### Embodiment 41

The composition of embodiment 40, wherein said composition is for the treatment of ADA-SCID and said transgene expresses adenosine deaminase (ADA).

### Embodiment 42

The composition of embodiment 40, wherein said composition is for the treatment of X-SCID and said transgene expresses IL-2 receptor gamma (IL-2Rγ).

### Embodiment 43

The composition of embodiment 40, wherein said composition is for the treatment of sickle cell disease and said transgene expresses an anti-sickling human β-globin gene.

### Embodiment 44

The composition of embodiment 43, wherein said anti-sickling human β-globin gene includes about 2.3 kb of recombinant human β-globin gene including exons and introns under the control of the human β-globin gene 5′ promoter and the human β-globin 3′ enhancer.

### Embodiment 45

The composition of embodiment 44, wherein said β-globin gene includes β-globin intron 2 with a 375 bp RsaI deletion from IVS2, and a composite human β-globin locus control region including HS2, HS3, and HS4.

### Embodiment 46

The composition according to any one of embodiments 40-45, wherein said host cell is a CD34⁺ cell.

### Embodiment 47

The composition of embodiment 46, wherein said host cell is a CD34⁺/CD38⁻ cell.

### Embodiment 48

A method for treating a subject for a pathology shown in column A below, including introducing into said subject progenitor or stem cells transfected with a vector according to any one of embodiments 2-23, wherein said vector contains one or more transgenes for the treatment of said pathology as shown in column B below:

| A | B |
|---|---|
| Pathology | Transgene/gene product |
| ADA-SCID | adenosine deaminase (ADA) |
| X-SCID | IL-2 receptor gamma (IL-2Rγ) |
| PNP-SCID | PNP gene |
| JAK3 | Janus kinase-3 (JAK3) |
| Artemis/DCLRE1C | Artemis gene |
| Sickle Cell Disease | anti-sickling human β-globin gene |
| Haemophilia A | Factor VIII |
| Haemophilia B | Factor IX |
| Cystic fibrosis | CFTR |
| Muscular Dystrophy | full length or shortened dystrophin |
| Adrenoleukodystrophy (ALD) | ABCD1 gene |
| Parkinson's Disease | TH, AADC, and GCH1 |
| Phenylketonuria | phenylalanine hydroxylase (PAH) |
| Aspartylglucosaminuria | Aspartylglucosaminidase |
| Fabry | α-Galactosidase A |
| Infantile Batten Disease | Palmitoyl Protein Thioesterase |
| Classic Late Infantile Batten Disease | Tripeptidyl Peptidase |
| Juvenile Batten Disease (CNL2) | Lysosomal Transmembrane Protein |
| Cystinosis | Cysteine transporter |
| Farber | Acid ceramidase |
| Fucosidosis | Acid α-L-fucosidase |
| Galactosidosialidosis | Protective protein/cathepsin A |
| Gaucher types 1, 2, and 3 | Acid β-glucosidase |
| GMl gangliosidosis | Acid β-galactosidase |
| Hunter | Iduronate-2-sulfatase |
| Hurler-Scheie | α-L-Iduronidase |
| Krabbe | Galactocerebrosidase. |
| α-Mannosidosis | Acid α-mannosidase. |
| β-Mannosidosis | Acid β-mannosidase |
| Maroteaux-Lamy | Arylsulfatase B |
| Metachromatic leukodystrophy | Arylsulfatase A |
| Morquio A | N-Acetylgalactosamine-6-sulfate |
| Morquio B | Acid β-galactosidase |
| Mucolipidosis II/III | N-Acety lglucosamine-1 -phospho- |
| | transferase |
| Niemann-PickA, B | Acid sphingomyelinase (aSM) |
| Niemann-Pick C | NPC-1 |
| Pompe Acid | α-glucosidase |
| Sandhoff | β-Hexosaminidase B |
| Sanfilippo A | Heparan N-sulfatase |
| Sanfilippo B | α-N-Acetylglucosaminidase |
| Sanfilippo C | Acetyl-CoA: α-glucosaminide |
| Sanfilippo D | N-Acetylglucosamine-6-sulfate |
| Schindler Disease | α-N-Acetylgalactosaminidase |
| Schindler-Kanzaki. | α-N-Acetylgalactosaminidase |
| Sialidosis | α-Neuramidase |
| Sly | β-Glucuronidase |
| Tay-Sachs | β-Hexosaminidase A |
| Wolman | Acid Lipase. |

### Embodiment 49

The method of embodiment 48, wherein said method is for the treatment of ADA-SCID and said transgene expresses adenosine deaminase (ADA).

### Embodiment 50

The method of embodiment 48, wherein said method is for the treatment of X-SCID and said transgene expresses IL-2 receptor gamma (IL-2Rγ).

### Embodiment 51

The method of embodiment 48, wherein said method is for the treatment of sickle cell disease and said transgene expresses an anti-sickling human β-globin gene.

### Embodiment 52

The method of embodiment 51, wherein said anti-sickling human β-globin gene includes about 2.3 kb of recombinant human β-globin gene including exons and introns under the control of the human β-globin gene 5′ promoter and the human β-globin 3′ enhancer.

### Embodiment 53

The method of embodiment 52, wherein said β-globin gene includes β-globin intron 2 with a 375 bp RsaI deletion from IVS2, and a composite human β-globin locus control region including HS2, HS3, and HS4.

### Embodiment 54

The method according to any one of embodiments 48-53, wherein said introducing includes transducing a stem cell and/or progenitor cell from said subject with said vector; and transplanting said transduced cell or cells derived therefrom into said subject where said cells or derivatives therefrom express said transgene.

### Embodiment 55

The method according to any one of embodiments 48-54, wherein, wherein the cell is a progenitor cell.

### Embodiment 56

The method according to any one of embodiments 48-54, wherein the cell is a stem cell.

### Embodiment 57

The method according to any one of embodiments 48-56, wherein said cell is a derived from bone marrow.

### Embodiment 58

The method according to any one of embodiments 48-57, wherein said cell is a CD34⁺ cell.

### Embodiment 59

The method of embodiment 58, wherein said cell is a CD34⁺/CD38⁻ cell.

### Embodiment 60

The method according to any one of embodiments 48-59, wherein said cell is derived from said subject.

### Embodiment 61

A population of cells that provide improved transduction with a recombinant lentivirus, said population of cells being enriched for CD34⁺/CD38⁻ cells.

### Embodiment 62

The population of cells of embodiment 61, wherein said CD34+/CD38− cells are derived from blood or bone marrow.

### Embodiment 63

The population of according to any one of embodiments 61-62, wherein said CD34+/CD38− cells are transfected with a retroviral vector containing a transgene.

### Embodiment 64

The population of cells of embodiment 63, wherein said CD34+/CD38− cells are transduced with a retroviral vector selected from group consisting of an HIV-1 lentiviral vector, an HIV-2 lentiviral vector, an alpharetroviral vector, an equine infectious anemia virus (EIAV) lentiviral vector, an MoMLV vector, an X-MLV vector, a P-MLV vector, a A-MLV vector, a GALV vector, an HEV-W vector, an SIV-1 vector, an FIV-1 vector, and an SERV-1-5 vector.

### Embodiment 65

The population of cells of embodiment 63, wherein said CD34+/CD38− cells are transduced with a lentiviral vector.

### Embodiment 66

The population of cells of embodiment 65, wherein said CD34+/CD38− cells are transduced with a TAT-independent and self-inactivating (SIN) lentiviral vector.

### Embodiment 67

The population of cells according to any one of embodiments 63-66, wherein said transgene is a transgene to treat a pathology listed in Table 1.

### Embodiment 68

The population of cells according to any one of embodiments 63-66, wherein said transgene encodes ADA, IL-2γR, or an antisickling gene.

### Embodiment 69

The population of cells of embodiment 63, wherein said cells are transfected with a CCL-βAS3-FB LV.

### Embodiment 70

A method of improving transduction of stem cells or progenitor cells including providing for said transduction a population of stem cells or progenitor cells that are enriched for CD34+/CD38− cells.

### Definitions

“Recombinant” is used consistently with its usage in the art to refer to a nucleic acid sequence that comprises portions that do not naturally occur together as part of a single sequence or that have been rearranged relative to a naturally occurring sequence. A recombinant nucleic acid is created by a process that involves the hand of man and/or is generated from a nucleic acid that was created by hand of man (e.g., by one or more cycles of replication, amplification, transcription, etc.). A recombinant virus is one that comprises a recombinant nucleic acid. A recombinant cell is one that comprises a recombinant nucleic acid.

As used herein, the term “recombinant lentiviral vector” or “recombinant LV) refers to an artificially created polynucleotide vector assembled from an LV and a plurality of additional segments as a result of human intervention and manipulation.

By “globin nucleic acid molecule” is meant a nucleic acid molecule that encodes a globin polypeptide. In various embodiments the globin nucleic acid molecule may include regulatory sequences upstream and/or downstream of the coding sequence.

By “globin polypeptide” is meant a protein having at least 85%, or at least 90%, or at least 95%, or at least 98% amino acid sequence identity to a human alpha, beta or gamma globin.

The term “therapeutic functional globin gene” refers to a nucleotide sequence the expression of which leads to a globin that does not produce a hemoglobinopathy phenotype, and which is effective to provide therapeutic benefits to an individual with a defective globin gene. The functional globin gene may encode a wild-type globin appropriate for a mammalian individual to be treated, or it may be a mutant form of globin, preferably one which provides for superior properties, for example superior oxygen transport properties or anti-sickling properties. The functional globin gene includes both exons and introns, as well as globin promoters and splice donors/acceptors.

By “an effective amount” is meant the amount of a required agent or composition comprising the agent to ameliorate or eliminate symptoms of a disease relative to an untreated patient. The effective amount of composition(s) used to practice the methods described herein for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

### BRIEF DESCRIPTION OF THE DRAWINGS

FIG. 1 schematically illustrates one embodiment of a reverse orientation UBC lentiviral vector (pCCLc-roUBC) transfer plasmid map. Dotted lines indicate plasmid backbone sequence outside of lentiviral sequences.

FIG. 2 illustrates expression from forward orientation CCLc-UBC-EmGFP vector compared to improved reverse orientation CCLc-roUBC-EmGFP vector.

FIG. 3 schematically illustrates the expression vectors used for studies. Lentiviral diagram depicts location in CCLc vectors. roUBC and roUBCs vectors contain a bovine growth hormone polyadenylation signal (not depicted), in the proper reverse orientation, after the end of the EmGFP reading frame. pCafe expression plasmids contained identical cassettes upstream of an SV40 polyadenylation signal.

FIG. 4, panels A and B, illustrates a genetic analysis of UBC splicing. Panel A: PCR strategy with primer locations and expected product sizes. Panel B: Electrophoresis of PCR products from controls and gDNA from cells transduced with lentiviral vectors bearing UBC promoter variants.

FIG. 5, panels A-C, illustrates a quantitative analysis of UBC intron loss during packaging and transduction. Panel A: Duplex ddPCR strategy for quantifying UBC intron copies (FAM-UBC intron), normalized to total proviral integrations (HEX-LV psi). Panel B: Representative raw data from ddPCR, illustrating separation between positive and negative droplets. Panel C: Ratio of UBC intron copies to total proviral copies in controls and samples transduced with LV bearing UBC promoter variants. Error bars represent 95% confidence interval based on ddPCR Poisson statistics.

FIG. 6, panels A-C, illustrates a flow cytometric expression analysis of UBC promoter variants. Panel A: Geometric mean fluorescence intensity (gMFI) of 293T cells 48 h posttransfection with expression plasmids. Error bars represent standard deviation of three biological replicates. UBC versus UBCs unpaired t-test P=0.0122, roUBC versus roUBCs P=0.0134. Panel B: gMFI of K562 cells 10 days post-transduction with CCLc lentiviral vectors bearing UBC promoter variants. Data are representative of multiple experiments. Panel C: gMFI of 293T cells 48 h post-transfection. Error bars represent SD of three biological replicates. * indicates P<0.05. UBC versus dEnh unpaired t-test P=0.0267, UBCs versus dEnh P=0.0008.

FIG. 7, panels A-E, illustrates EEF1A1 analysis. Panel A: Diagrams of lentiviral vectors bearing EEF1A1 promoter variants. Panel B: Gel electrophoresis of PCR product amplifying across EEF1A1 intron in stably transduced K562 cells, greater than 2 weeks post-transduction. Panel C: ddPCR quantification of the ratio of intron copies to proviral copies in samples analyzed in (panel B). Panel D: gMFI of transiently transfected 293T cells 48 h post-transfection with expression plasmids, measured by flow cytometry. Error bars in panel C represent 95% confidence interval, and in panels D and E represent SD of three biological replicates. * indicates P<0.05. Unpaired t-test P=0.0064. (E) gMFI of stably transduced K562 cells 10 days post-transduction, measured by flow cytometry.

FIG. 8 panels A-C, illustrated bidirectional vector analysis. Panel A: Vector schematics. Panel B: ddPCR analysis of intron loss in BD and roBD vectors. Panel C: gMFI of stably transduced 293T cells 2 weeks post-transduction, measured by flow cytometry. Error bars represent 95% confidence interval.

FIG. 9 illustrates digital PCR quantification of spliced vector junctions in viral vector supernatant and transduced K562 cells.

FIG. 10 illustrates expression of EmGFP measured by flow cytometry in myeloid cells differentiated from transduced human CD34+ HSPCs enriched from mobilized peripheral blood of a healthy donor. Expression was analyzed 10 days after transduction in populations that were approximately 10% transduced. Twotailed t-test p-value 0.0013.

FIG. 11 illustrates digital PCR quantification of UBC intron in proviral forms in transduced CD34+ HSPCs 10 days after transduction.

FIG. 12 illustrates results of a luciferase assay for enhancer activity of intron sequences in pGL4.25-based plasmids. Luciferase activity was measured in cell lysates 48 hours after transfection of 293T cells.

FIG. 13, panels A-C, illustrates expression and genetic analysis of UBC and EEF1A1 lentiviral vectors with introns swapped. Panel A: Expression analysis of K562 cells transduced with lentiviral vectors containing the indicated promoters, measured by flow cytometry 7 days after transduction. Panel B: Genetic analysis using primers diagrammed in FIG. 4, panel A. Product of intermediate length in three lanes on the right is a non-specific product from K562 genomic DNA and should be ignored. Panel C: Genetic analysis of transduced K562 cells using primers diagrammed in FIG. 7, panel A.

FIG. 14, panels A-B, illustrates isolation and growth properties of human CD34⁺ and CD34⁺/CD38⁻ cells. Panel A: Flow cytometry of CD34⁻ enriched cells showing gating strategy used to define CD34⁺/CD38⁺ cells (region P5) and CD34⁺/CD38⁻ cells (region P3). Panel B: Cell expansion from CD34⁺ and CD34⁺/CD38⁻ cells from cord blood. Cells were cocultured with irradiated MS5 stromal cells in long-term culture medium. The mean fold increase over cell number plated on day 0 is shown at each time point of long-term culture. Data represent cell expansion±SEM over time (n=3, p<0.0001). Abbreviation: APC, allophycocyanin.

FIG. 15, panels A-F, illustrates nalysis of transduction of CD34⁺ and CD34⁺/CD38⁻ cells with the CCL-β^{AS3}-FB LV vector. Panel A: Vector copy number (VCN)±SEM in transduced CD34⁺ and CD34⁺/CD38⁻ cells (n=9, p=0.02). Panel B: Distribution of hematopoietic colony types (n=80 colonies) formed by nontransduced cord blood (CB) CD34⁺ (NT-CD34⁺), transduced CD34⁺ (CD34⁺), and CD34⁺/CD38⁻ cells. Panel C: Percentage of plated NT-CD34⁺, CD34⁺, and CD34⁺/CD38⁻ cells that grew into hematopoietic colonies in vitro. Values represent the mean±SD. Panel D: Single CFU grown from transduced CD34⁺ (left) and CD34⁺/CD38⁻ (right) CB cells were analyzed for VCN by ddPCR (n=80 colonies). Graph indicates percentages of the CFU that were negative for vector by digital PCR (0 VC/cell) or that had VC/cell of 1-2, 3-4, 5-6, or >6. Panel E: Vector transduction dose-response for CD34⁺ and CD34⁺/CD38⁻ cells (n=3, p=0.05 at 6.6×10⁶ TU/ml, p=0.002 at 2×10⁷ TU/ml). Panel F: VCN over time in long-term culture (±SEM [n=3]) (time trend difference p=0.03, VCN difference p=0.004, linear mixed model). Asterisk indicates significance, *, p≤0.05; **, p≤0.01.

FIG. 16, panels A-C, illustrates analysis of transduction of CD34⁺ and CD34⁺/CD38⁻ cells by the CCL-MND-GFP LV vector. Panel A: Comparison of mean vector copy number±SEM after transduction with a dose range of CCL-MND-GFP LV analyzed by qPCR at day 14 of culture. Panel B: Representative histogram showing relative GFP expression of transduced CD34⁺ and CD34⁺/CD38⁻ cells. Panel C: Percentages of GFP1 cells determined by flow cytometry in CCL-MND-GFP-transduced CD34⁺ and CD34⁺/CD38⁻ cells (n=6, p=0.02). Abbreviation: GFP, green fluorescent protein.

FIG. 17, panels A-B, illustrates erythroid differentiation of CD34⁺ and CD34⁺/CD38⁻ cells transduced by the CCL-β^{AS3}-FB LV vector. Panel A: Comparison of vector copy number (VCN)±SEM during differentiation, at day 14 after transduction (n=3). Panel B: Percentage of HBBAS3 mRNA expression of all β-globin transcripts per VCN (% AS3/VCN) in erythroid cells differentiated from transduced CD34⁺ and CD34⁺/CD38⁻ cells analyzed by qRT-PCR (n=3).

FIG. 18, panels A-E, illustrates the role of vector envelope and receptor on transduction by the CCL-β^{AS3}-FB LV vector. LDL receptor expression by CD34⁺ and CD34⁺/CD38⁻ cells on: (panel A) freshly isolated CD34⁺ cells, (panel B) CD34⁺ cells at 48 hours of culture in cytokines, (panel C) freshly isolated CD34⁺/CD38⁻ cells, and (panel D) CD34⁺/CD38⁻ cells at 48 hours of culture in cytokines. Panel E: Transduction of CD34⁺ and CD34⁺/CD38⁻ cells with the RD114 pseudotyped CCL-β^{AS3}-FB LV vector. The graph represents the mean vector copy number of CD34⁺ and CD34⁺/CD38⁻ cells±SEM analyzed by qPCR at day 14 of culture (n=3). Abbreviation: LDL, low density lipoprotein.

FIG. 19, panels A and B, shows a comparison of engraftment of NOD. Cg-Prkd^{scid}Il2rg^{tm1wjil}/SzJ (NSG) mice. Panel A: Contribution to human CD451 cell engraftment in NSG mice by transduced, transplanted cell populations. Mock mice were transplanted with nontransduced human CB CD34⁺ cells; control mice were transplanted with transduced CD34⁺ cells; all other mice were transplanted with a combination of CD34⁺/CD38⁻ (1%) and CD34⁺/CD38⁺ cells (99%). Vectors used for transduction (CCLc-UBC-mStrawberry-FB, CCLc-UBC-mCitrine-FB, and CCLc-UBCmCerulean-FB LV) were alternated among the cell populations for each transplant. BM harvested from NSG mice with human cell engraftment (% huCD451/% huCD4511muCD451 cells) was further analyzed for percent vector expression using flow cytometry. (B): Vector copy number (VCN) of cells analyzed in vivo mouse transplantation. In vivo VCN was analyzed from BM harvested 80-90 days after transplantation into NSG mice using ddPCR with primers and probes specific to each fluorescent reporter. The in vivo VCN/mouse for each population of cells is displayed separately.

FIG. 20, panels A-C, illustrates an analysis of CD34⁺ and CD34⁺/CD38⁻ cells transduction with the CCL-β^{AS3}-FB LV vector and hematopoietic potential at day 30 of long-term culture. Panel A: Distribution of hematopoietic colony types formed by non-transduced (NT)-CD34⁺ cells (n=37 colonies), transduced CD34⁺ cells (n=29 colonies) and CD34⁺/CD38⁻ cells (n=81 colonies). Panel B: Percentage of plated NT-CD34⁺, transduced CD34⁺ and CD34⁺/CD38⁻ cell that grew into hematopoietic colonies in vitro. Values represent the mean±SD; asterisk indicates significance, ****p≤0.0001. Panel C: VCN distribution of in vitro single CFU grown from transduced CD34⁺ analyzed by ddPCR (n=22 colonies). Graph indicates percentages of the CFU that were negative for vector (0 VC/cell) or that had VC/cell of 1-2, 3-4, 5-6 or >6. VCN distribution for in vitro CFU grown from transduced CD34⁺/CD38⁻ cells (n=43 colonies).

FIG. 21, panels A-C, illustrates an analysis of CD34⁺ and CD34⁺/CD38⁻ cells transduction with the CCL-β^{AS3}-FB LV vector and hematopoietic potential at day 60 of long-term culture. Panel A: Distribution of hematopoietic colony types formed by non-transduced (NT)-CD34⁺ cells (n=5 colonies), transduced CD34⁺ cells (n=3 colonies) and CD34⁺/CD38⁻ cells (n=22 colonies). Panel B: Percentage of plated NT-CD34⁺, transduced CD34⁺ and CD34⁺/CD38⁻ cell that grew into hematopoietic colonies in vitro. Panel C: VCN distribution of in vitro single CFU grown from transduced CD34⁺/CD38⁻ cells analyzed by ddPCR (n=18 colonies). Graph indicates percentages of the CFU that were negative for vector (0 VC/cell) or that had VC/cell of 1-2, 3-4, 5-6 or >6.

FIG. 22, panels A-C, illustrates erythroid differentiation of CD34⁺ and CD34⁺/CD38⁻ cells transduced by the CCL-β^{AS3}-FB LV vector. Flow cytometry analysis of cells from erythroid cultures from (panel A) unfractionated CD34⁺ cell and (panel B) CD34+/CD38− cells. Enucleated erythrocytes are present in the left upper quadrant as DRAQ5 negative, glycophorin A (GpA) positive cells. Panel C: Percentage of enucleated RBC at the end of erythroid differentiation.

FIG. 23 illustrates the contribution to total human engraftment in NSG mice by transduced, transplanted cell populations. Mock mice were transplanted with non-transduced human CB CD34⁺ cells; control mice were transplanted with transduced CD34⁺ cells; all other mice were transplanted with a combination of CD34⁺/CD38⁻ (1%) and CD34⁺/CD38⁺ cells (99%). Vectors used for transduction (CCLc-UBC-mStrawberry-FB, CCLc-UBC-mCitrine-FB and CCLc-UBC-mCerulean-FB LV) were alternated among the cell populations for each transplant. BM harvested from NSG mice with human cell engraftment (% huCD45⁺/% huCD45⁺+muCD45⁺ cells) was further analyzed for percent vector expression using flow cytometry.

### DETAILED DESCRIPTION

The HIV-1-based lentiviral vector (LV) is one of the most common tools used for genetic modifications in biological experiments and in gene therapy. Most LVs used are self-inactivating, meaning that the region within the long terminal repeat containing the promoter and enhancers has been removed (Zufferey et al. (1998) J Virol., 72: 9873-9880). In order to express a transgene within such a vector, a promoter must therefore be placed within the vector payload along with the transgene. Typically, in order to express a protein-coding gene, a heterologous RNA Pol II viral or cellular promoter will be used, and common examples are viral promoters from cytomegalovirus, murine leukaemia virus, and spleen focus-forming virus, and cellular promoters from human genes such as elongation factor 1 alpha (EEF1A1), ubiquitin C (UBC) and phosphoglycerate kinase (PGK1) (Schambach et al. (2006) Mol. Ther., 13, 391-400; Dull et al. (1998) J. Viral., 72: 8463-8471).

During the viral production process, RNA Pol II transcribes the vector genome, typically from a transfer plasmid that has been transfected into the producer cells. Virtually all systems incorporate the Rev protein from HIV-1, which binds to the Rev response element (RRE) within the HIV-1 genome and mediates splicing-independent nuclear export of the viral genome. Despite the incorporation of the RRE sequence into LV constructs, however, introns within the vector payload can be lost during packaging if the splicing event retains the packaging signal (Psi) in the transcript. With some expression cassettes, though, such as one including the intron-containing promoter of EEF1A1 and one containing the hybrid CAG promoter, intron loss has not been observed during lentiviral packaging (Ramezani et al. (2000) Mol. Ther., 2: 458-469; Zaiss et al. (2002) J. Virol., 76: 7209-7219). From these observations, it has sometimes been inferred that lentiviral gene transfer allows for the transmission of introns (Logan et al. (2002) Curr. Opin. Biotechnol., 13: 429-436).

We set out to investigate whether the intron contained by the human UBC promoter is faithfully transmitted from a transfer plasmid through to proviral forms in stably transduced cells. We hypothesized that a loss of the UBC intron would result in a significant reduction in transgene expression, as the UBC intron has been reported to possess strong enhancer activity (Bianchi et al. (2009) Gene, 448: 88-101). In contrast to previous findings with the EEF1A1 intron, the UBC intron was found to be missing in the majority of proviral forms in cells transduced with vector produced from intron-containing plasmids. The lack of the UBC intron resulted in a roughly 2-fold decrease in expression in both transient transfection and stable transduction experiments in cell lines, and a 4-fold decrease in transduction experiment in primary cells. This contrasted strikingly with experiments with the EEF1A1 promoter, in which the majority of proviral forms maintained the intron. Reversal of the UBC expression cassette prevented this splicing-mediated intron loss and maximized expression in uni- and bidirectional LVs. Without being bound by a particular theory, it is believed the difference in intron maintenance between the UBC and EEF1A1 promoters is caused by promoter exonic sequences, rather than the intronic sequences themselves.

In view of the foregoing, in various embodiments, recombinant retroviral vectors are provided comprising a human ubiquitin C (UBC) promoter where the UBC promoter is in a reverse orientation in the vector so that the direction of transcription from the promoter is oriented towards a 5′ long terminal repeat (LTR) of the vector. In various embodiments the vector comprises a multiple cloning site located so that a gene/cDNA inserted in the multiple cloning site is operably linked to the reverse orientation UBC so that the direction of transcription of the gene controlled by the promoter is oriented towards a 5′ long terminal repeat (LTR) of said vector.

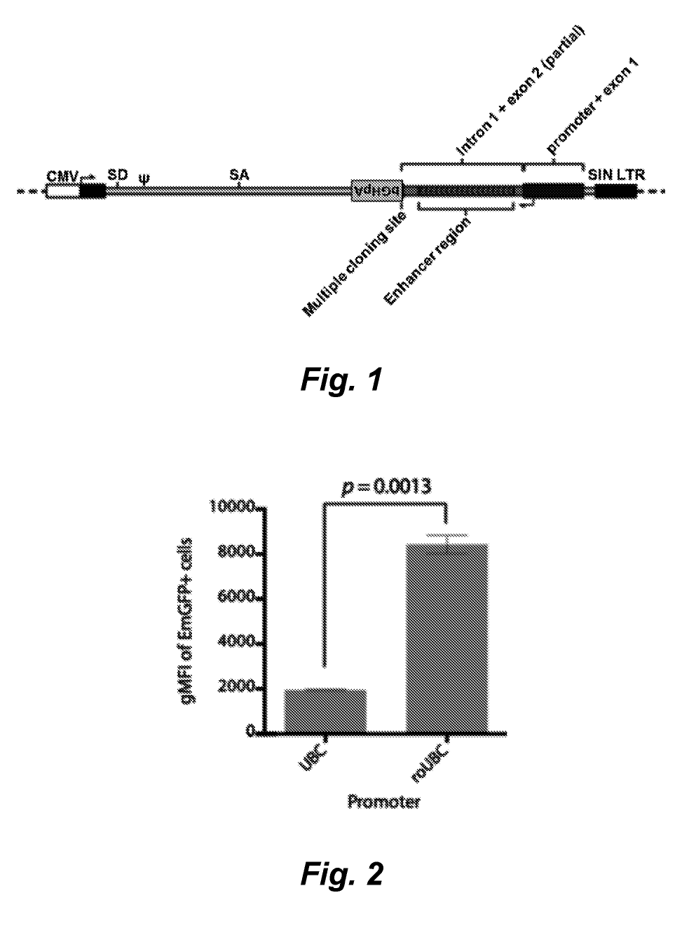

By way of non-limiting illustration, one such viral vector is illustrated in FIG. 1. In order to make this lentiviral vector “pCCLc-roUBC”, a fragment from the human ubiquitin C gene (UCSC human genome sequence version hg19, minus strand from position 125398318 to position 125399530) was inserted into the multiple cloning site of pCCLc using standard molecular cloning techniques such as restriction digestion and ligation, or assembly techniques In-Fusion, Gibson assembly, or sequence- and ligation-independent cloning (SLIC).

The direction of insertion is such that the direction of transcription from the UBC promoter is oriented towards the 5′ long terminal repeat (LTR) of the pCCLc vector, unlike typical lentiviral vectors which have the direction of transcription oriented towards the 3′ LTR.

Upon lentiviral transduction of target cells, this roUBC vector expresses transgenes at an approximately four-fold higher level than vectors with a UBC promoter oriented such that transcription progresses towards the 3′ LTR. This value was determined in human hematopoietic stem and progenitor cells transduced with vectors encoding the Emerald variant of the green fluorescent protein (EmGFP) transgene (FIG. 2).

The utility of this promoter orientation is not limited to the pCCLc lentiviral vector, and is believed to be beneficial in other retroviral vectors as well. Accordingly in certain embodiments, other retroviral vectors comprising a reverse orientation UBC promoter are contemplated. Such vectors include, but are not limited to an HIV-2 lentiviral vector, an alpharetroviral vector, an equine infectious anemia virus (EIAV) lentiviral vector, an MoMLV vector, an X-MLV vector, a P-MLV vector, a A-MLV vector, a GALV vector, an HEV-W vector, an SIV-1 vector, an FIV-1 vector, an SERV-1-5 vector, and the like.

In various embodiments the vector additional contains a polyadenylation signal (polyA) inserted in the same (reverse) orientation 3′ of the promoter fragment (5′ of the promoter, with respect to the entire vector sequence) in order to effect efficient polyadenylation of the transgene. Suitable polyadenylation signals include, but are not limited to bovine growth hormone polyA, human growth hormone polyA, a rabbit β-globin gene polyadenylation signal, a human herpes virus (HSV) polyadenylation signal, a thymidine kinase (TK) gene polyadenylation signal, and the like.

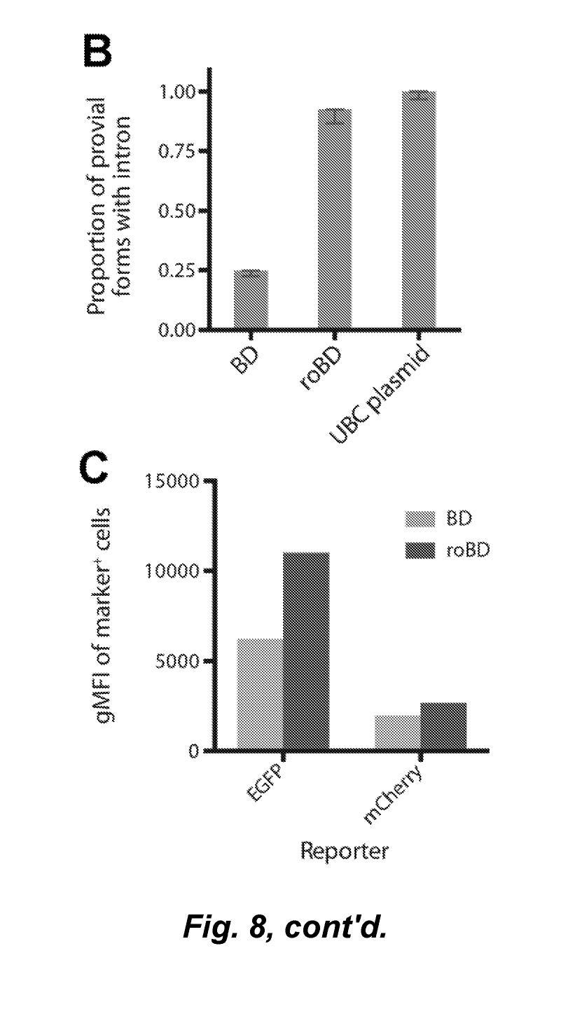

It was also discovered that the reversal of the UBC promoter also improves expression from bidirectional vectors, such as the one described in U.S. Pat. No. 8,501,464 B2. This was demonstrated by the increased expression of EGFP in a bidirectional vector with a reversed UBC promoter, “roBD”, compared to the forward orientation counterpart, “BD” (see, e.g., FIG. 8, panel C). Accordingly, in certain embodiments, bidirectional retroviral vectors (e.g., bidirectional lentiviral vectors) comprising a human UBC gene in reverse orientation are also contemplated.

In various embodiments viral particles comprising the vectors described herein are also contemplated as well as host cells (e.g., stem cells, progenitor cells, etc.) transduced with the vectors described herein. In certain embodiments the host cell is a CD34+ hematopoietic stem cell. As described herein in Example 2, it was discovered that using isolated CD34⁺/CD38⁻ permits the use of significantly less vector and appears to improve transduction for HSC gene therapy. Accordingly in certain embodiments, the host cell is a CD34⁺/CD38⁻ cell and in certain embodiments a population of cells enriched for CD34+/CD38− cells is provided.

The use of CD34+/CD38− cells need not be limited to transduction with vectors containing a reverse orientation UBC. In certain embodiments such cells can be transduced with essentially any retroviral vector (e.g., an anti-sickling retroviral vector such as CCL-βAS3-FB LV described in PCT Publication No: WO2014043131 A1 (PCT/US2013/059073)).

In various embodiments, compositions for the treatment of a pathology are provided where the composition comprises a stem cell and/or progenitor cell transfected with a vector as described herein where the vector contains one or more transgenes for the treatment of the pathology (e.g., as shown in Table 1, below) where the composition additionally comprises a pharmaceutically acceptable carrier.

In certain embodiments method of treating a pathology (e.g., a pathology that can be treated by introduction of a transgene are contemplated. In certain embodiments the methods comprise introducing into a subject having or at risk for the pathology progenitor or stem cells transfected with a vector described herein where the vector contains one or more transgenes for the treatment of the pathology (e.g., as shown in Table 1, below).

It will be noted that while the discussion provided below is with respect to SIN lentiviral vectors, using the various elements, constructs, and teachings provided herein other retroviral vectors containing a reverse orientation UBC promoter or a bidirectional promoter comprising a reverse orientation UBC promoter can readily be produced.

### TAT-Independent and Self Inactivating Lentiviral Vectors.

As noted above, it is contemplated that the reverse orientation UBC promoter can be used in essentially any retroviral vector. In certain embodiments the retroviral vector is a lentiviral vector (LV) and in certain embodiments the lentiviral vectors (LVs) comprises a TAT-independent, self-inactivating (SIN) configuration. Thus, in various embodiments it is desirable to employ in the LVs described herein an LTR region that has reduced promoter activity relative to wild-type LTR. Such constructs can be provided that are effectively “self-inactivating” (SIN) which provides a biosafety feature. SIN vectors are ones in which the production of full-length vector RNA in transduced cells is greatly reduced or abolished altogether. This feature minimizes the risk that replication-competent recombinants (RCRs) will emerge. Furthermore, it reduces the risk that that cellular coding sequences located adjacent to the vector integration site will be aberrantly expressed.

Furthermore, a SIN design reduces the possibility of interference between the LTR and the promoter that is driving the expression of the transgene. SIN LVs can often permit full activity of the internal promoter.

The SIN design increases the biosafety of the LVs. The majority of the HIV LTR is comprised of the U3 sequences. The U3 region contains the enhancer and promoter elements that modulate basal and induced expression of the HIV genome in infected cells and in response to cell activation. Several of these promoter elements are essential for viral replication. Some of the enhancer elements are highly conserved among viral isolates and have been implicated as critical virulence factors in viral pathogenesis. The enhancer elements may act to influence replication rates in the different cellular target of the virus

As viral transcription starts at the 3′ end of the U3 region of the 5′ LTR, those sequences are not part of the viral mRNA and a copy thereof from the 3′ LTR acts as template for the generation of both LTR's in the integrated provirus. If the 3′ copy of the U3 region is altered in a retroviral vector construct, the vector RNA is still produced from the intact 5′ LTR in producer cells, but cannot be regenerated in target cells. Transduction of such a vector results in the inactivation of both LTR's in the progeny virus. Thus, the retrovirus is self-inactivating (SIN) and those vectors are known as SIN transfer vectors.

In certain embodiments self-inactivation is achieved through the introduction of a deletion in the U3 region of the 3′ LTR of the vector DNA, i.e., the DNA used to produce the vector RNA. During RT, this deletion is transferred to the 5′ LTR of the proviral DNA. Typically, it is desirable to eliminate enough of the U3 sequence to greatly diminish or abolish altogether the transcriptional activity of the LTR, thereby greatly diminishing or abolishing the production of full-length vector RNA in transduced cells. However, it is generally desirable to retain those elements of the LTR that are involved in polyadenylation of the viral RNA, a function typically spread out over U3, R and U5. Accordingly, in certain embodiments, it is desirable to eliminate as many of the transcriptionally important motifs from the LTR as possible while sparing the polyadenylation determinants.

The SIN design is described in detail in Zufferey et al. (1998) J Virol. 72(12): 9873-9880, and in U.S. Pat. No. 5,994,136. As described therein, there are, however, limits to the extent of the deletion at the 3′ LTR. First, the 5′ end of the U3 region serves another essential function in vector transfer, being required for integration (terminal dinucleotide+att sequence). Thus, the terminal dinucleotide and the att sequence may represent the 5′ boundary of the U3 sequences which can be deleted. In addition, some loosely defined regions may influence the activity of the downstream polyadenylation site in the R region. Excessive deletion of U3 sequence from the 3′LTR may decrease polyadenylation of vector transcripts with adverse consequences both on the titer of the vector in producer cells and the transgene expression in target cells.

Additional SIN designs are described in U.S. Patent Publication No: 2003/0039636. As described therein, in certain embodiments, the lentiviral sequences removed from the LTRs are replaced with comparable sequences from a non-lentiviral retrovirus, thereby forming hybrid LTRs. In particular, the lentiviral R region within the LTR can be replaced in whole or in part by the R region from a non-lentiviral retrovirus. In certain embodiments, the lentiviral TAR sequence, a sequence which interacts with TAT protein to enhance viral replication, is removed, preferably in whole, from the R region. The TAR sequence is then replaced with a comparable portion of the R region from a non-lentiviral retrovirus, thereby forming a hybrid R region. The LTRs can be further modified to remove and/or replace with non-lentiviral sequences all or a portion of the lentiviral U3 and U5 regions.

Accordingly, in certain embodiments, the SIN configuration provides a retroviral LTR comprising a hybrid lentiviral R region that lacks all or a portion of its TAR sequence, thereby eliminating any possible activation by TAT, wherein the TAR sequence or portion thereof is replaced by a comparable portion of the R region from a non-lentiviral retrovirus, thereby forming a hybrid R region. In a particular embodiment, the retroviral LTR comprises a hybrid R region, wherein the hybrid R region comprises a portion of the HIV R region (e.g., a portion comprising or consisting of the nucleotide sequence shown in SEQ ID NO: 10 in US 2003/0039636) lacking the TAR sequence, and a portion of the MoMSV R region (e.g., a portion comprising or consisting of the nucleotide sequence shown in SEQ ID NO: 9 in 2003/0039636) comparable to the TAR sequence lacking from the HIV R region. In another particular embodiment, the entire hybrid R region comprises or consists of the nucleotide sequence shown in SEQ ID NO: 11 in 2003/0039636.

Suitable lentiviruses from which the R region can be derived include, for example, HIV (HIV-1 and HIV-2), EIV, SIV and FIV. Suitable retroviruses from which non-lentiviral sequences can be derived include, for example, MoMSV, MoMLV, Friend, MSCV, RSV and Spumaviruses. In one illustrative embodiment, the lentivirus is HIV and the non-lentiviral retrovirus is MoMSV.

In another embodiment described in US 2003/0039636, the LTR comprising a hybrid R region is a left (5′) LTR and further comprises a promoter sequence upstream from the hybrid R region. Preferred promoters are non-lentiviral in origin and include, for example, the U3 region from a non-lentiviral retrovirus (e.g., the MoMSV U3 region). In one particular embodiment, the U3 region comprises the nucleotide sequence shown in SEQ ID NO: 12 in US 2003/0039636. In another embodiment, the left (5′) LTR further comprises a lentiviral U5 region downstream from the hybrid R region. In one embodiment, the U5 region is the HIV U5 region including the HIV att site necessary for genomic integration. In another embodiment, the U5 region comprises the nucleotide sequence shown in SEQ ID NO: 13 in US 2003/0039636. In yet another embodiment, the entire left (5′) hybrid LTR comprises the nucleotide sequence shown in SEQ ID NO: 1 in US 2003/0039636.

In another illustrative embodiment, the LTR comprising a hybrid R region is a right (3′) LTR and further comprises a modified (e.g., truncated) lentiviral U3 region upstream from the hybrid R region. The modified lentiviral U3 region can include the att sequence, but lack any sequences having promoter activity, thereby causing the vector to be SIN in that viral transcription cannot go beyond the first round of replication following chromosomal integration. In a particular embodiment, the modified lentiviral U3 region upstream from the hybrid R region consists of the 3′ end of a lentiviral (e.g., HIV) U3 region up to and including the lentiviral U3 att site. In one embodiment, the U3 region comprises the nucleotide sequence shown in SEQ ID NO: 15 in US 2003/0039636. In another embodiment, the right (3′) LTR further comprises a polyadenylation sequence downstream from the hybrid R region. In another embodiment, the polyadenylation sequence comprises the nucleotide sequence shown in SEQ ID NO: 16 in US 2003/0039636. In yet another embodiment, the entire right (5′) LTR comprises the nucleotide sequence shown in SEQ ID NO: 2 or 17 of US 2003/0039636.

Thus, in the case of HIV based LV, it has been discovered that such vectors tolerate significant U3 deletions, including the removal of the LTR TATA box (e.g., deletions from −418 to −18), without significant reductions in vector titers. These deletions render the LTR region substantially transcriptionally inactive in that the transcriptional ability of the LTR in reduced to about 90% or lower.

It has also been demonstrated that the trans-acting function of Tat becomes dispensable if part of the upstream LTR in the transfer vector construct is replaced by constitutively active promoter sequences (see, e.g., Dull et al. (1998) J Virol. 72(11): 8463-8471. Furthermore, we show that the expression of rev in trans allows the production of high-titer HIV-derived vector stocks from a packaging construct which contains only gag and pol. This design makes the expression of the packaging functions conditional on complementation available only in producer cells. The resulting gene delivery system, conserves only three of the nine genes of HIV-1 and relies on four separate transcriptional units for the production of transducing particles.

In one embodiments illustrated in Example 1, the cassette expressing an anti-sickling β-globin (e.g., βAS3) is placed in the pCCL LV backbone, which is a SIN vector with the CMV enhancer/promoter substituted in the 5′ LTR.

It will be recognized that the CMV promoter typically provides a high level of non-tissue specific expression. Other promoters with similar constitutive activity include, but are not limited to the RSV promoter, and the SV40 promoter. Mammalian promoters such as the beta-actin promoter, ubiquitin C promoter, elongation factor lapromoter, tubulin promoter, etc., may also be used.

The foregoing SIN configurations are illustrative and non-limiting. Numerous SIN configurations are known to those of skill in the art. As indicated above, in certain embodiments, the LTR transcription is reduced by about 95% to about 99%. In certain embodiments LTR may be rendered at least about 90%, at least about 91%, at least about 92%, at least about 93%, at least about 94%, at least about 95% at least about 96%, at least about 97%, at least about 98%, or at least about 99% transcriptionally inactive.

### Insulator Element

In certain embodiments, to further enhance biosafety, insulators are inserted into the vectors described herein. Insulators are DNA sequence elements present throughout the genome. They bind proteins that modify chromatin and alter regional gene expression. The placement of insulators in the vectors described herein offer various potential benefits including, inter alia: 1) Shielding of the vector from positional effect variegation of expression by flanking chromosomes (i.e., barrier activity); and 2) Shielding flanking chromosomes from insertional trans-activation of gene expression by the vector (enhancer blocking). Thus, insulators can help to preserve the independent function of genes or transcription units embedded in a genome or genetic context in which their expression may otherwise be influenced by regulatory signals within the genome or genetic context (see, e.g., Burgess-Beusse et al. (2002) Proc. Natl. Acad. Sci. USA, 99: 16433; and Zhan et al. (2001) Hum. Genet., 109: 471). In the present context insulators may contribute to protecting lentivirus-expressed sequences from integration site effects, which may be mediated by cis-acting elements present in genomic DNA and lead to deregulated expression of transferred sequences. In various embodiments LVs are provided in which an insulator sequence is inserted into one or both LTRs or elsewhere in the region of the vector that integrates into the cellular genome.

The first and best characterized vertebrate chromatin insulator is located within the chicken β-globin locus control region. This element, which contains a DNase-I hypersensitive site-4 (cHS4), appears to constitute the 5′ boundary of the chicken β-globin locus (Prioleau et al. (1999) EMBO J. 18: 4035-4048). A 1.2-kb fragment containing the cHS4 element displays classic insulator activities, including the ability to block the interaction of globin gene promoters and enhancers in cell lines (Chung et al. (1993) Cell, 74: 505-514), and the ability to protect expression cassettes in Drosophila (Id.), transformed cell lines (Pikaart et al. (1998) Genes Dev. 12: 2852-2862), and transgenic mammals (Wang et al. (1997) Nat. Biotechnol., 15: 239-243; Taboit-Dameron et al. (1999) Transgenic Res., 8: 223-235) from position effects. Much of this activity is contained in a 250-bp fragment. Within this stretch is a 49-bp cHS4 core (Chung et al. (1997) Proc. Natl. Acad. Sci., USA, 94: 575-580) that interacts with the zinc finger DNA binding protein CTCF implicated in enhancer-blocking assays (Bell et al. (1999) Cell, 98: 387-396).

One illustrative and suitable insulator is FB (FII/BEAD-A), a 77 bp insulator element, that contains the minimal CTCF binding site enhancer-blocking components of the chicken β-globin 5′ HS4 insulators and a homologous region from the human T-cell receptor alpha/delta blocking element alpha/delta I (BEAD-I) insulator described by Ramezani et al. (2008) Stem Cell 26: 3257-3266. The FB “synthetic” insulator has full enhancer blocking activity. This insulator is illustrative and non-limiting. Other suitable insulators may be used including, for example, the full length chicken beta-globin HS4 or insulator sub-fragments thereof, the ankyrin gene insulator, and other synthetic insulator elements.

### Packaging Signal.

In various embodiments the vectors described herein further comprise a packaging signal. A “packaging signal,” “packaging sequence,” or “psi sequence” is any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art.

### Rev Responsive Element (RRE).

In certain embodiments the vectors described herein comprise a Rev response element (RRE) to enhance nuclear export of unspliced RNA. RREs are well known to those of skill in the art. Illustrative RREs include, but are not limited to RREs such as that located at positions 7622-8459 in the HIV NL4-3 genome (Genbank accession number AF003887) as well as RREs from other strains of HIV or other retroviruses. Such sequences are readily available from Genbank or from the database with URL hiv-web.lanl.gov/content/index.

Central PolyPurine Tract (cPPT).

In various embodiments the vectors described herein further include a central polypurine tract. Insertion of a fragment containing the central polypurine tract (cPPT), e.g., in lentiviral (e.g., HIV-1) vector constructs is known to enhance transduction efficiency drastically, reportedly by facilitating the nuclear import of viral cDNA through a central DNA flap.

### Expression-Stimulating Posttranscriptional Regulatory Element (PRE)

In certain embodiments the vectors described herein may comprise any of a variety of posttranscriptional regulatory elements (PREs) whose presence within a transcript increases expression of the heterologous nucleic acid (e.g., ADA, IL-2Rγ, βAS3, and the like) at the protein level. PREs may be particularly useful in certain embodiments, especially those that involve lentiviral constructs with modest promoters.

One type of PRE is an intron positioned within the expression cassette, which can stimulate gene expression. However, introns can be spliced out during the life cycle events of a lentivirus. Hence, if introns are used as PRE's they are typically placed in an opposite orientation to the vector genomic transcript.

Posttranscriptional regulatory elements that do not rely on splicing events offer the advantage of not being removed during the viral life cycle. Some examples are the posttranscriptional processing element of herpes simplex virus, the posttranscriptional regulatory element of the hepatitis B virus (HPRE) and the woodchuck hepatitis virus (WPRE). Of these the WPRE is typically preferred as it contains an additional cis-acting element not found in the HPRE. This regulatory element is typically positioned within the vector so as to be included in the RNA transcript of the transgene, but outside of stop codon of the transgene translational unit.

The WPRE is characterized and described in U.S. Pat. No. 6,136,597. As described therein, the WPRE is an RNA export element that mediates efficient transport of RNA from the nucleus to the cytoplasm. It enhances the expression of transgenes by insertion of a cis-acting nucleic acid sequence, such that the element and the transgene are contained within a single transcript. Presence of the WPRE in the sense orientation was shown to increase transgene expression by up to 7 to 10 fold. Retroviral vectors transfer sequences in the form of cDNAs instead of complete intron-containing genes as introns are generally spliced out during the sequence of events leading to the formation of the retroviral particle. Introns mediate the interaction of primary transcripts with the splicing machinery. Because the processing of RNAs by the splicing machinery facilitates their cytoplasmic export, due to a coupling between the splicing and transport machineries, cDNAs are often inefficiently expressed. Thus, the inclusion of the WPRE in a vector results in enhanced expression of transgenes.

### Transduced Host Cells and Methods of Cell Transduction.

The recombinant vectors and resulting virus described herein are capable of transferring a nucleic acid sequence (e.g., a nucleic acid encoding an anti-sickling β-globin, ADA, IL-2Rγ gene, any of the other targets/transgenes listed in Table 1, and the like) into a mammalian cell. For delivery to cells, vectors of the present invention are can be used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

Typically, the vectors are introduced via transfection into the packaging cell line. The packaging cell line produces viral particles that contain the vector genome. Methods for transfection are well known by those of skill in the art. After cotransfection of the packaging vectors and the transfer vector to the packaging cell line, the recombinant virus is recovered from the culture media and tittered by standard methods used by those of skill in the art. Thus, the packaging constructs can be introduced into human cell lines by calcium phosphate transfection, lipofection or electroporation, generally together with a dominant selectable marker, such as neomycin, DHFR, Glutamine synthetase, followed by selection in the presence of the appropriate drug and isolation of clones. In certain embodiments the selectable marker gene can be linked physically to the packaging genes in the construct.

Stable cell lines where the packaging functions are configured to be expressed by a suitable packaging cell are known (see, e.g., U.S. Pat. No. 5,686,279, which describes packaging cells). In general, for the production of virus particles, one may employ any cell that is compatible with the expression of lentiviral Gag and Pol genes, or any cell that can be engineered to support such expression. For example, producer cells such as 293T cells and HT1080 cells may be used.

The packaging cells with a retroviral vector (e.g., a lentiviral vector) incorporated in them form producer cells. Producer cells are thus cells or cell-lines that can produce or release packaged infectious viral particles carrying the therapeutic gene of interest (e.g., anti-sickling β-globin, ADA, IL-2Rγ gene, etc.). These cells can further be anchorage dependent which means that these cells will grow, survive, or maintain function optimally when attached to a surface such as glass or plastic. Some examples of anchorage dependent cell lines used as lentiviral vector packaging cell lines when the vector is replication competent are HeLa or 293 cells and PERC.6 cells.

Accordingly, in certain embodiments, methods are provided of delivering a gene to a cell which is then integrated into the genome of the cell, comprising contacting the cell with a virion containing a lentiviral vector described herein. The cell (e.g., in the form of tissue or an organ) can be contacted (e.g., infected) with the virion ex vivo and then delivered to a subject (e.g., a mammal, animal or human) in which the gene (e.g., anti-sickling β-globin, ADA, IL-2Rγ gene, etc.) will be expressed. In various embodiments the cell can be autologous to the subject (i.e., from the subject) or it can be non-autologous (i.e., allogeneic or xenogenic) to the subject. Moreover, because the vectors described herein are capable of being delivered to both dividing and non-dividing cells, the cells can be from a wide variety including, for example, bone marrow cells, mesenchymal stem cells (e.g., obtained from adipose tissue), and other primary cells derived from human and animal sources. Alternatively, the virion can be directly administered in vivo to a subject or a localized area of a subject (e.g., bone marrow).

Of course, as noted above, the vectors described herein are particularly useful in the transduction of human hematopoietic progenitor cells or a hematopoietic stem cells, obtained either from the bone marrow, the peripheral blood or the umbilical cord blood, as well as in the transduction of a CD4⁺ T cell, a peripheral blood B or T lymphocyte cell, and the like. In certain embodiments particularly preferred targets are CD34⁺ cells. In certain embodiments the targets are CD34⁺/CD38⁻ cells.

### Gene Therapy.

In certain embodiments the vectors described herein are useful for introducing transgenes into subjects e.g., to treat a pathology that can be ameliorated by correction of a genetic defect and/or by expression of one or more heterologous gene(s). In one illustrative, but non-limiting embodiment, the method involves contacting a population of human cells that include hematopoietic stem cells with a vector described herein comprising the transgene(s) of interest under conditions to effect the transduction of a human stem cell or progenitor cell in the population by the vector. The stem cells may be transduced in vivo or in vitro, depending on the ultimate application. Even in the context of human gene therapy, such as gene therapy of human stem cells, one may transduce the stem cell or progenitor cell in vivo or, alternatively, transduce in vitro followed by infusion of the transduced cell(s) into a human subject. In one aspect the human cells can be removed from a human, e.g., a human patient, using methods well known to those of skill in the art and transduced as noted above. The transduced cells are then reintroduced into the same or a different human where expression of the transgene(s) ameliorates one or more symptoms of the pathology, or effectively cures the pathology, or slows the progression or the pathology.

Pathologies and Targets for Gene Therapy.

The vectors described herein are useful for the delivery of transgenes in the treatment of essentially any condition that can be treated using gene therapy techniques. can be used to deliver transgenes for the treatment of a number of pathologies. In this regard, it is noted that a large number gene therapy clinical protocols are approved or in review (see, e.g., Misra (2013) J. A. P. I., 61: 127-133, and the like).

In certain embodiments the vectors contain a transgene for the treatment of a pathology such as SCID, sickle cell disease, a liposomal storage disease, cystic fibrosis, muscular dystrophy, phenylketonuria, Parkinson's disease, or haemophilia. An illustrative, but non-limiting, list of pathologies and associated “targets” that may be treated with gene transfer (e.g., gene therapy) methods using the vectors described herein are shown in Table 1.

TABLE 1
Illustrative, but non-limiting examples of pathologies treatable
by gene therapy and associated target/gene product.

| Pathology | Target Gene/Gene Product |
|---|---|
| SCID | |
| ADA-SCID | adenosine deaminase (ADA) |
| X-SCID | IL-2 receptor gamma (IL-2Rγ) |
| PNP-SCID | purine nucleoside phosphorylase (PNP) |
| | gene |
| JAK3 | Janus kinase-3 (JAK3) |
| Artemis/DCLRE1C | Artemis gene |
| Sickle Cell Disease | anti-sickling human β-globin gene |
| Haemophilia | |
| Haemophilia A | Factor VIII |
| Haemophilia B | Factor IX |
| Cystic fibrosis | CFTR |
| Muscular Dystrophy | full length or shortened dystrophin |
| Adrenoleukodystrophy (ALD) | ABCD1 gene |
| Parkinson's Disease | TH, AADC, and GCH1 to medium |
| | spiny neurons (MSN) in the |
| | striatum, to induce ectopic dopamine |
| | synthesis from tyrosine |
| Familial hypercholesterolemia | Low-density-lipoprotein receptor |
| Fanconi's anemia | Complement group C gene |
| Gaucher's disease | Glucocerebrosidase gene |
| Alpha-1-antitrypsin deficiency | Alpha-1-antitrypsin gene |
| Phenylketonuria | phenylalanine hydroxylase (PAH) |
| Liposomal storage diseases | |
| Aspartylglucosaminuria | Aspartylglucosaminidase |
| Fabry | α-Galactosidase A |
| Infantile Batten Disease | Palmitoyl Protein Thioesterase |
| Classic Late Infantile Batten | Tripeptidyl Peptidase |
| Disease (CLN1) | |
| Juvenile Batten Disease | Lysosomal Transmembrane Protein |
| (CNL2) | |
| Cystinosis | Cysteine transporter |
| Farber | Acid ceramidase |
| Fucosidosis | Acid α-L-fucosidase |
| Galactosidosialidosis | Protective protein/cathepsin A |
| Gaucher types 1, 2, and 3 | Acid β-glucosidase |
| GMl gangliosidosis | Acid β-galactosidase |
| Hunter | Iduronate-2-sulfatase |
| Hurler-Scheie | α-L-Iduronidase |
| Krabbe | Galactocerebrosidase. |
| α-Mannosidosis | Acid α-mannosidase. |
| β-Mannosidosis | Acid β-mannosidase |
| Maroteaux-Lamy | Arylsulfatase B |
| Metachromatic leukodystrophy | Arylsulfatase A |
| Morquio A | N-Acetylgalactosamine-6-sulfate |
| Morquio B | Acid β-galactosidase |
| Mucolipidosis II/III | N-Acety lglucosamine-1 - |
| | phosphotransferase |
| Niemann-PickA, B | Acid sphingomyelinase (aSM) |
| Niemann-Pick C | NPC-1 |
| Pompe Acid | α-glucosidase |
| Sandhoff | β-Hexosaminidase B |
| Sanfilippo A | Heparan N-sulfatase |
| Sanfilippo B | α-N-Acetylglucosaminidase |
| Sanfilippo C | Acetyl-CoA: α-glucosaminide |
| Sanfilippo D | N-Acetylglucosamine-6-sulfate |
| Schindler Disease | α-N-Acetylgalactosaminidase |
| Schindler-Kanzaki. | α-N-Acetylgalactosaminidase |
| Sialidosis | α-Neuramidase |
| Sly | β-Glucuronidase |
| Tay-Sachs | β-Hexosaminidase A |
| Wolman | Acid Lipase |

In one illustrative embodiment the vectors described herein are used to treat ADA-SCID by introduction of an adenosine deaminase (ADA) gene/cDNA. In another embodiment, the vectors described herein are used for the treatment of X-SCID by introduction of an IL-2 receptor gamma (IL-2γ) gene. In certain embodiments vectors described herein are useful for the treatment of sickle cell disease by the introduction of an anti-sickling human β-globin gene (e.g., as described in PCT Publication No: WO2014043131 A1 (PCT/US2013/059073).

The foregoing pathologies and targets are illustrative and non-limiting. Using the teachings provided herein, the vectors described herein can be used to deliver any of a large number of genes/cDNAs.

Stem Cell/Progenitor Cell Gene Therapy.

In various embodiments the vectors described herein are particularly useful for the transduction of human hematopoietic progenitor cells or haematopoietic stem cells (HSCs), obtained either from the bone marrow, the peripheral blood or the umbilical cord blood, as well as in the transduction of a CD4⁺ T cell, a peripheral blood B or T lymphocyte cell, and the like. In certain embodiments particularly preferred targets are CD34⁺ cells. In certain embodiments preferred targets are CD34⁺/CD38⁻ cells.

When cells, for instance CD34⁺ cells, CD34⁺/CD38⁻ cells, dendritic cells, peripheral blood cells or tumor cells are transduced ex vivo, the vector particles are incubated with the cells using a dose generally in the order of between 1 to 50 multiplicities of infection (MOI) which also corresponds to 1×10⁵ to 50×10⁵ transducing units of the viral vector per 10⁵ cells. This of course includes amount of vector corresponding to 1, 2, 3, 4, 5, 6, 7, 8, 9, 10, 15, 20, 25, 30, 35, 40, 45, and 50 MOI. Typically, the amount of vector may be expressed in terms of HeLa transducing units (TU).

It is noted that as shown in Example 1 in PCT Publication No: WO2014043131 A1 (PCT/US2013/059073), a dose-related increase in gene transfer achieved (the average VC/cell measured by qPCR) was found only for vector concentrations below 2×10⁷ TU/ml. Higher vector concentrations did not increase the transduction efficacy and, in fact, often had a negative effect on the extent of transduction (data not shown). Based on these findings, the CCL-βAS3-FB vector was used at a standard concentration of 2×10⁷ TU/ml (MOI=40).

In certain embodiments cell-based therapies involve providing stem cells and/or hematopoietic precursors, transduce the cells with the virus encoding the transgene of interest (e.g., an anti-sickling human β-globin, and then introduce the transformed cells into a subject in need thereof (e.g., a subject with the sickle cell mutation).

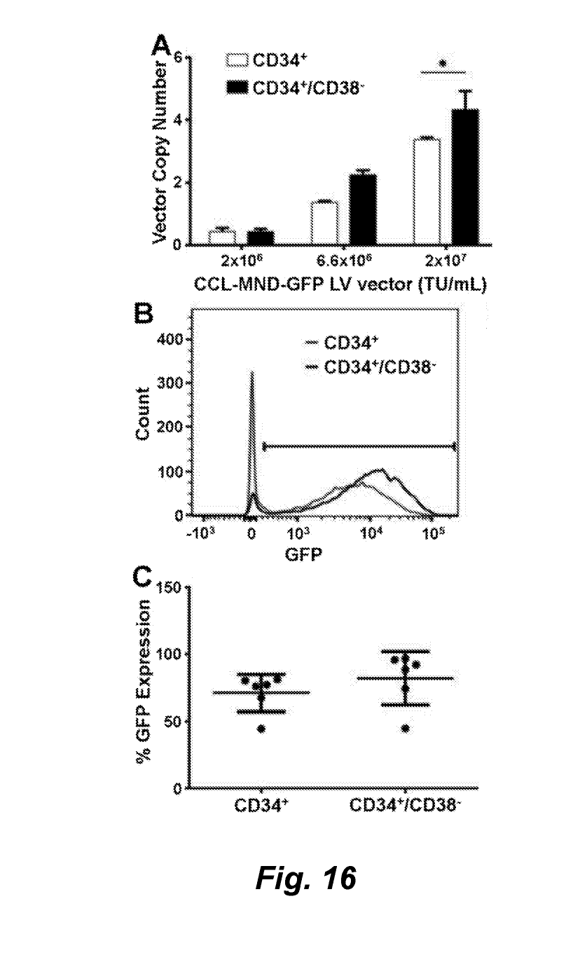

In certain embodiments the methods involve isolating population of cells, e.g., stem cells from a subject, optionally expand the cells in tissue culture, and administer the lentiviral vector whose presence within a cell results in production of an anti-sickling β-globin in the cells in vitro. The cells are then returned to the subject, where, for example, they may provide a population of red blood cells that produce the anti-sickling β globin see, e.g., FIG. 16 in in PCT Publication No: WO2014043131 A1 (PCT/US2013/059073).

In some embodiments of the invention, a population of cells, which may be cells from a cell line or from an individual other than the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

Where stem cells are to be used, it will be recognized that such cells can be derived from a number of sources including bone marrow (BM), cord blood (CB) CB, mobilized peripheral blood stem cells (mPBSC), and the like. In certain embodiments the use of induced pluripotent stem cells (IPSCs) is contemplated. Methods of isolating hematopoietic stem cells (HSCs), transducing such cells and introducing them into a mammalian subject are well known to those of skill in the art.

Direct Introduction of Vector.

In certain embodiments direct treatment of a subject by direct introduction of the vector is contemplated. The vector compositions may be formulated for delivery by any available route including, but not limited to parenteral (e.g., intravenous), intradermal, subcutaneous, oral (e.g., inhalation), transdermal (topical), transmucosal, rectal, and vaginal. Commonly used routes of delivery include inhalation, parenteral, and transmucosal.

In various embodiments pharmaceutical compositions can include an vector in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

In some embodiments, active agents, i.e., a vector described herein and/or other agents to be administered together with the vector, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art. Suitable materials can also be obtained commercially from Alza Corporation and Nova Pharmaceuticals, Inc. Liposomes can also be used as pharmaceutically acceptable carriers. These can be prepared according to methods known to those skilled in the art, for example, as described in U.S. Pat. No. 4,522,811. In some embodiments the composition is targeted to particular cell types or to cells that are infected by a virus. For example, compositions can be targeted using monoclonal antibodies to cell surface markers, e.g., endogenous markers or viral antigens expressed on the surface of infected cells.

It is advantageous to formulate compositions in dosage unit form for ease of administration and uniformity of dosage. Dosage unit form as used herein refers to physically discrete units suited as unitary dosages for the subject to be treated; each unit comprising a predetermined quantity of a vector (e.g., an LV) calculated to produce the desired therapeutic effect in association with a pharmaceutical carrier.

A unit dose need not be administered as a single injection but may comprise continuous infusion over a set period of time. Unit dose of the vector(s) described herein may conveniently be described in terms of transducing units (T.U.) of vector, as defined by titering the vector on a cell line such as HeLa or 293. In certain embodiments unit doses can range from 10³, 10⁴, 10⁵, 10⁶, 10⁷, 10⁸, 10⁹, 10¹⁰, 10¹¹, 10¹², 10¹³ T.U. and higher.

Pharmaceutical compositions can be administered at various intervals and over different periods of time as required, e.g., one time per week for between about 1 to about 10 weeks; between about 2 to about 8 weeks; between about 3 to about 7 weeks; about 4 weeks; about 5 weeks; about 6 weeks, etc. It may be necessary to administer the therapeutic composition on an indefinite basis. The skilled artisan will appreciate that certain factors can influence the dosage and timing required to effectively treat a subject, including but not limited to the severity of the disease or disorder, previous treatments, the general health and/or age of the subject, and other diseases present. Treatment of a subject with a vector (e.g., LV) as contemplated herein, can include a single treatment or, in many cases, can include a series of treatments.

Illustrative doses for administration of gene therapy vectors and methods for determining suitable doses are known in the art. It is furthermore understood that appropriate doses of a vector may depend upon the particular recipient and the mode of administration. The appropriate dose level for any particular subject may depend upon a variety of factors including the pathology at issue, target tissues, age, body weight, general health, gender, and diet of the subject, the time of administration, the route of administration, the rate of excretion, other administered therapeutic agents, and the like.

In certain embodiments the vectors can be delivered to a subject by, for example, intravenous injection, local administration, or by stereotactic injection (see, e.g., Chen et al. (1994) Proc. Natl. Acad. Sci. USA, 91: 3054). In certain embodiments vectors may be delivered orally or via inhalation and may be encapsulated or otherwise manipulated to protect them from degradation, enhance uptake into tissues or cells, etc. Pharmaceutical preparations can include a vector in an acceptable diluent, or can comprise a slow release matrix in which a vector is imbedded. Alternatively or additionally, where a vector can be produced intact from recombinant cells, as is the case for retroviral vectors as described herein, a pharmaceutical preparation can include one or more cells that produce vectors. Pharmaceutical compositions comprising a vector described herein can be included in a container, pack, or dispenser, optionally together with instructions for administration.

The foregoing compositions, methods and uses are intended to be illustrative and not limiting. Using the teachings provided herein other variations on the compositions; methods and uses will be readily available to one of skill in the art.

### EXAMPLES

The following examples are offered to illustrate, but not to limit the claimed invention.

### Example 1

### Rescue of Splicing-Mediated Intron Loss Maximizes Expression in Lentiviral Vectors Containing the Human Ubiquitin C Promoter

Lentiviral vectors almost universally use heterologous internal promoters to express transgenes. One of the most commonly used promoter fragments is a 1.2-kb sequence from the human ubiquitin C (UBC) gene, encompassing the promoter, some enhancers, first exon, first intron and a small part of the second exon of UBC. Because splicing can occur after transcription of the vector genome during vector production, we investigated whether the intron within the UBC promoter fragment is faithfully transmitted to target cells. As described in this example, genetic analysis revealed that more than 80% of proviral forms lack the intron of the UBC promoter. The human elongation factor 1 alpha (EEF1A1) promoter fragment intron was not lost during lentiviral packaging, and this difference between the UBC and EEF1A1 promoter introns was conferred by promoter exonic sequences. UBC promoter intron loss caused a 4-fold reduction in transgene expression. Movement of the expression cassette to the opposite strand prevented intron loss and restored full expression. This increase in expression was mostly due to non-classical enhancer activity within the intron, and movement of putative intronic enhancer sequences to multiple promoter-proximal sites actually repressed expression. Reversal of the UBC promoter also prevented intron loss and restored full expression in bidirectional lentiviral vectors.

### Materials and Methods.

Plasmid Construction

All plasmid sequences used in these studies are included as in the accompanying Sequence listing which is incorporated herein by reference for all purposes.

The human ubiquitin C promoter was amplified from FUGW (Lois et al. (2002) Science, 295: 868-872), phosphorylated with T4 polynucleotide kinase and ligated into linearized and blunted pCafe (Cassette for expression) to generate pCafe-UBC. The woodchuck hepatitis virus post-transcriptional regulatory element sequence (herein ‘PRE,’ referred to as ‘LPRE’ in Schambach et al.) was polymerase chain reaction (PCR) amplified and cloned into pCafe-UBC linearized with KpnI using In-Fusion (Clontech Laboratories, Mountain View, Calif., USA, Cat. No. 639645). The Emerald variant of EGFP was PCR amplified from pRSET-EmGFP (Life Technologies, Carlsbad, Calif., USA, Cat. No. V353-20) and cloned into HpaI-linearized pCafe-UBC-PRE using In-Fusion to generate pCafe-UBC-EmGFP-PRE. pCafe-UBCs-EmGFP-PRE was generated in a similar fashion, with UBC cloning primers designed to omit the UBC intron sequence.

For the expression cassettes in the reverse orientation (ro) plasmids, pCafe-roUBC-EmGFP-bGHpA and pCafe-roUBCs-EmGFP-bGHpA, the bovine growth hormone polyadenylation signal sequence was amplified from pcDNA4/HisMax A (Life Technologies, Cat. No. V864-20) and inserted after the transgene.

For constructs with the UBC intron repositioned (i), pCafe-iUBC-EmGFP-PRE, pCafe-roiUBC-EmGFP-PRE and pCafe-rofiUBC-EmGFP-PRE, UBC intronic sequences were amplified from pCafe-UBC-PRE and cloned into EcoRV-linearized pCafe-UBCs-EmGFP-PRE using In-Fusion.

For a construct with the UBC enhancer deleted (dEnh), pCafe-dEnhUBC-EmGFP-PRE, pCafe-UBC-EmGFP-PRE was amplified using overlapping, outward-facing primers flanking the putative intronic enhancer region and recircularized with In-Fusion after DpnI treatment.

For all pCCLc (Dull et al. (1998) J. Viral., 72: 8463-8471) LVs, expression cassettes were removed from pCafe plasmids with EcoRV/KpnI digestion and ligated into EcoRV/KpnI-linearized pCCLc.

The bidirectional (BD) vector was constructed by assembly of PCR amplicons of bovine growth hormone polyA (bGHpA) and bidirectional mCMV/UBC promoter (Kamata et al. PLoS ONE, 5: e11834) and EGFP and WPRE from FUGW, and mCherry from EFS-single-IDLV (Joglekar et al. (2013) Mol. Ther., 21: 1705-1717) designed with overlapping homology with the pCCLc backbone using the In-Fusion Cloning Kit (Clontech, Mountain View, Calif., USA). The roBD vector was constructed by restriction digest of BD to invert the mCherry-bidirectional promoter-EGFP cassette between inverse bGHpA and WPRE, and ligated with NEB Quick Ligase Kit (New England Biolabs, Ipswitch, Mass., USA).

Cell Culture

D10 medium was prepared by adding 50-ml heat-inactivated fetal calf serum (Gemini Bio-Products, West Sacramento, Calif., USA, Cat. No. 900-208) and 5.5-ml 100×L-Glutamine:Penicillin:Streptomycin solution (Gemini Bio-Products Cat. No. 400-110) to 500-ml Dulbecco's modified Eagle's medium without L-glutamine (Mediatech, Herndon, Va., USA, Cat. No. 15-013-CV). R10 medium was prepared by adding the same two components to 500-ml RPMI 1640 medium without L-glutamine (Mediatech Cat. No. 15-040). 293T cells (ATCC, Manassas, Va., USA, Cat. No. CRL-1268) were maintained in D10 medium, and K562 (ATCC Cat. No. CCL-243) cells were maintained in R10 medium.

Vector Production

LV supernatant was produced by transfection of 1×107 HEK293T cells with 10-μg pCMVΔR8.91 (Zufferey et al. (1997) Nat. Biotechnol., 15: 871-875), 10 ng of the appropriate pCCLc vector plasmid and 2-μg pCAG-VSV-G (Hanawa et al. (2002) Mol. Ther., 5: 242-251). Transfection mixtures were prepared in 1.5-ml DPBS by adding the plasmids and 66-μl-mg/ml branched PEI solution (Sigma-Aldrich, St. Louis, Mo., USA, Cat. No. 408727-100ML), and then vortexing for several seconds. After incubation at room temperature for 5-10 min, transfection mixes were added dropwise to 293T cells plated 24 h earlier in 10-cm dishes. After ˜16 h, the medium was changed to UltraCULTURE medium (Lonza, Basel, Switzerland, Cat. No. 12-725F) supplemented with 50-U/ml penicillin, 50-μg/ml streptomycin, 2-mM L-glutamine and 20-mM HEPES. Viral supernatant was harvested 24-48 h after this medium change.

For roUBC vectors, 5-μg pcDNA3-NovB2 was included to prevent a drop in titers caused by the presence of transcripts antisense to the vector genomic RNA (Maetzig et al. (2010) Gene Ther., 17: 400-411). An additional 15-μl 1-μg/ml PEI solution was added to compensate for the increased plasmid DNA.

Vector was concentrated ˜150-fold by ultracentrifugation at 26 000 rpm for 90 min at 4° C. in a Beckman Coulter SW-32Ti rotor for transduction of human CD34+ HSPCs.

Transfection

293T cells were seeded at 8×10⁵ cells/well in 6-well plates (Corning, Corning, N.Y., USA, Cat. No. 3516) in D10 medium. Twenty-four hours later, 1.5 μg of plasmid was prepared for transfection in 200-μl Opti-MEM I medium (Life Technologies, Carlsbad, Calif., USA, Cat. No. 31985-062) in 1.5-ml microcentrifuge tubes. 4.5 μl of TransIT-293 transfection reagent (Mirus Bio, Madison, Wis., USA, Cat. No. MIR 2700) was added, and the mixtures were vortexed briefly and incubated at room temperature for 5 min before being added dropwise to the cells. Cells were collected 48 h after transfection by brief trypsinization and analyzed for green fluorescent protein reporter expression on a BDLSR Fortessa flow cytometer.

Transduction

For lentiviral expression analysis, K562 cells were plated in 24-well plates at 50 000 cells/well and treated with a range of vector doses to obtain populations with 10% transduction or lower, thus ensuring that the majority of cells received only single integrations. Cells were cultured for 1-2 weeks before flow cytometric analysis to dilute out non-integrated vector and to allow fluorescent protein levels to reach steady state.

For expression analysis in primary human CD34+ HSPCs from mobilized peripheral blood, cryopreserved cells were thawed and prestimulated overnight in X-VIVO 15 medium (Lonza) supplemented with 50-ng/ml human FLT-3 ligand, 50-ng/ml human stem cell factor and 50 ng/ml human thrombopoietin (PeproTech, Rocky Hill, N.J., USA). Viral vector was then added in an equal volume of the same medium to achieve a final vector concentration of 3×10⁵ transducing units/ml, as determined by transduction of K562 cells. This vector dose yielded ˜10% transduction. Twenty-four hours after vector addition, 2 ml of myeloid differentiation medium was added. This was composed of IMDM supplemented with 20% FBS, 0.5% bovine serum albumin, 5-ng/ml human interleukin-3, 10-ng/ml human interleukin-6 and 25-ng/ml human stem cell factor (PeproTech).

PCR Analysis of Splicing

Genomic DNA from transduced K562 cells was analyzed via PCR using KAPA HiFi Hot Start polymerase and primers UBC intron F (AAG TAG TCC CTT CTC GGC GAT, (SEQ ID NO:1)), UBC intron R (GGT CAG CTT GCC GTA GGT, (SEQ ID NO:2)), EEF1A1 intron F (GTT CTT TTT CGC AAC GGG TTT G, (SEQ ID NO:3)) and EEF1A1 intron R (TGT GGC CGT TTA CGT CGC, (SEQ ID NO:4)).

Quantitative droplet digital PCR (ddPCR) was carried out by analysis of genomic DNA from UBC vector-transduced K562 cells using primers UBCint F (GGC GAG TGT GTT TTG TGA AGT TT, (SEQ ID NO:5)) and EmGFP R (TAC GTC GCC GTC CAG CTC, (SEQ ID NO:6)), and probe FAM-EmGFP (FAM-CAC CAC CCC GGT GAA CAG CTC CTC G, (SEQ ID NO:7)). For EEF1A1 vector analysis, the UBCint F primer was substituted with EEF1A1int F (TCT CAA GCC TCA GAC AGT GGT, (SEQ ID NO:8)).

The spliced form of UBC was quantified using UBCs F (GCT GTG ATC GTC ACT TGA CA, (SEQ ID NO:9)) instead of UBCint F. ddPCR was carried out according to the manufacturer's instructions, using 100 ng of template gDNA. One unit of DraI enzyme (New England Biolabs) was added to the ddPCR master mix containing ddPCR Supermix for Probes (Bio-Rad, Hercules, Calif., USA), and predigestion was carried out in the PCR reaction mixes for 1-2 h at 37° C. before droplet generation and thermal cycling.

For analysis of vector genomes in vector supernatant, RNA was purified from 500 μl of raw vector supernatant using the PureLink RNA Mini Kit liquid sample procedure (Life Technologies). Reverse transfection was carried out before PCR using iScript cDNA Synthesis Kit (Bio-Rad).

Luciferase Assay

pGL4.25 vector (Promega, Madison, Wis., USA) containing an optimized luciferase ORF driven by a minimal TATA box promoter was used to assay for enhancer activity of the UBC and EEF1A1 introns. A promoterless enhancer sequence from the CMV promoter was used as a positive control. All inserts were cloned via PCR and Gibson assembly into pGL4.25 linearized with EcoRV and KpnI. Luciferase assays were performed in 293T cells plated on 96 well tissue culture-treated plates. Fifty thousand cells per well were plated in D10 medium, and 18 h later, transfection mixes were prepared in OPTI-MEM with 100-μg reporter plasmid and 0.3-μl TransIT-293 per well. Samples were prepared 48 h after transfection with the Dual-Luciferase Reporter Assay System (Promega) and luminescence readings were taken with a Tecan Infinite M1000 PRO plate reader (Tecan, Mannedorf, Switzerland).

### Results.

UBC Intron is Missing from Proviral Forms, and Expression Cassette Reversal Prevents Loss

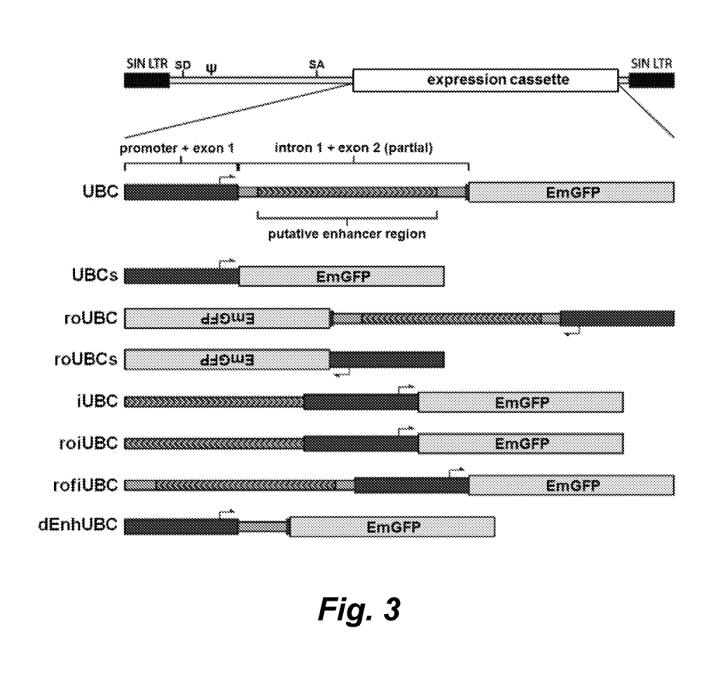

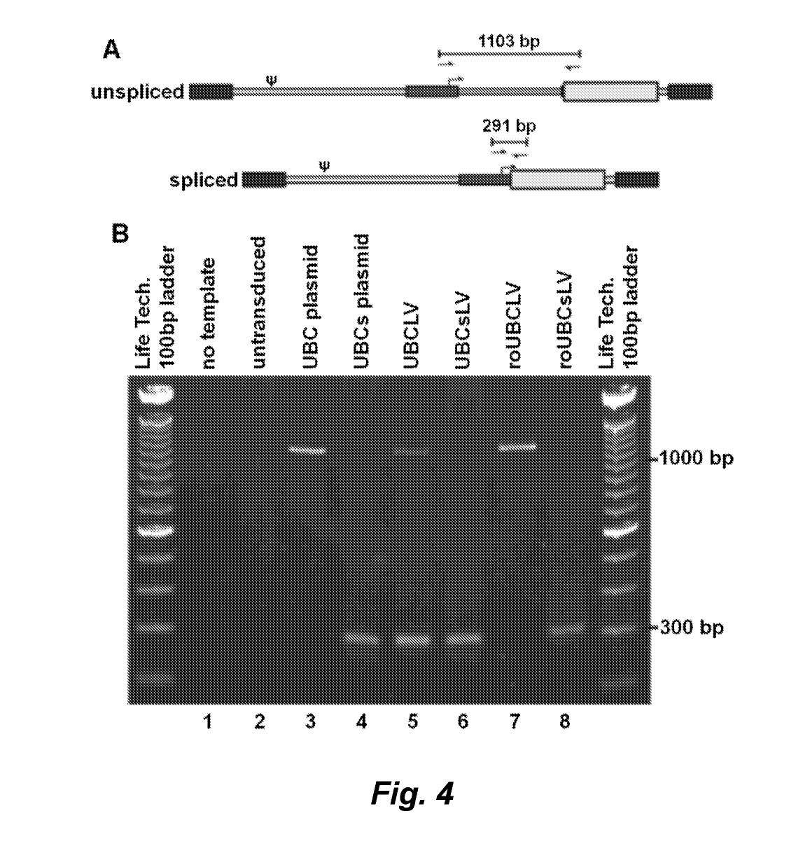

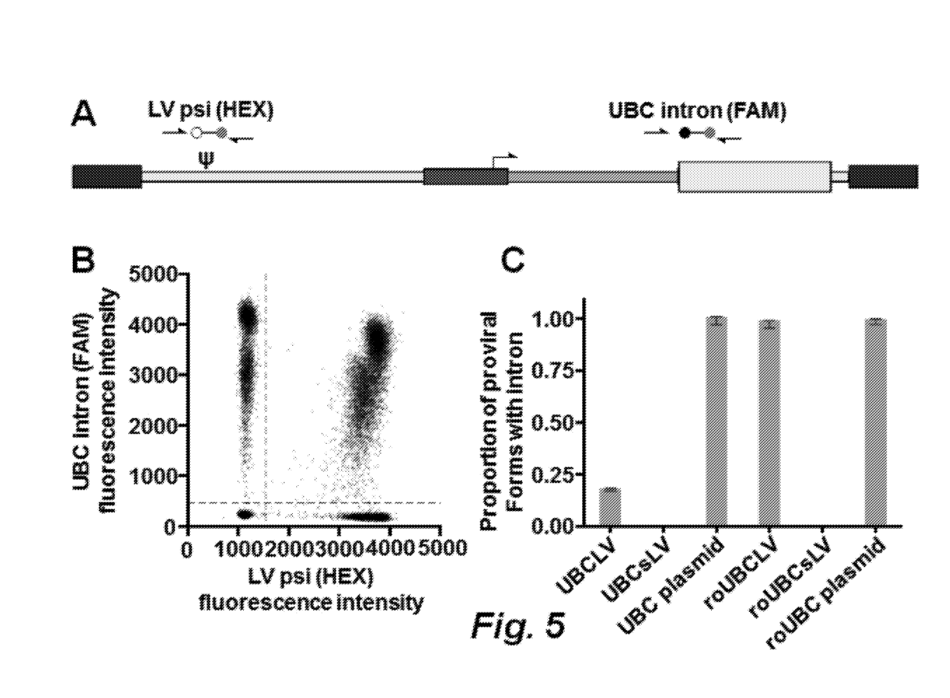

To assess whether UBC intron 1 is maintained during packaging, pCCLc LV DNA constructs and simpler pCafe expression plasmid constructs for transient transfection were created with various modifications of the UBC promoter (FIG. 3). All constructs contained the Emerald variant of green fluorescent protein (EmGFP), which allowed for expression analysis via flow cytometry (Tsien (1998) Annu. Rev. Biochem. 67: 509-544). UBC constructs contained the full UBC promoter fragment, as it exists in the human genome, whereas shorter UBCs constructs were designed with a full deletion of UBC intron 1, which would be the expected proviral form if canonical splicing occurred during packaging. To test whether movement of the expression cassette to the opposite strand would avoid splicing-mediated loss of the intron, reverse orientation (ro) constructs roUBC and roUBCs were created by reversing the promoter and transgene and inserting a polyadenylation signal after the transgene. Importantly, while the payloads of the pCCLc LVs pass through an RNA intermediate stage and are susceptible to splicing-mediated loss, payloads of the pCafe expression plasmids have no RNA intermediate and can therefore not lose genetic elements due to splicing. Viral vectors were produced in 293T cells and used to transduce K562 cells for PCR-based genetic analysis of proviral forms (FIG. 4, panel A). PCR analysis of gDNA two weeks post-transduction revealed that many CCLc-UBCEmGFP-PRE proviral forms contained an amplicon consistent with intron loss, as indicated by analysis of UBCs proviral forms (FIG. 4, panel B, lanes 5 and 6). Sanger sequencing of the short product confirmed that the expected canonical splicing had occurred (data not shown). In contrast, roUBC proviral forms yielded no truncated PCR product, suggesting that reversal of the expression cassette fully prevented intron loss (FIG. 4, panel B, lane 7). Because of the significant difference in predicted PCR product size between the intron-containing templates and intron-lacking templates, there could be a substantial bias toward amplification of the intron-lacking templates and overestimation of the amount of intron loss from this result. Therefore, to quantify the frequency of intron loss a duplex digital PCR assay was set up, in which the signal from a primer and probe set spanning the intron and EmGFP transgene was normalized using a primer and probe set to the LV packaging signal (FIG. 5, panels A and B). This analysis showed that only 18% of UBC vector forms retained the UBC intron (FIG. 5, panel C), while roUBC vector forms fully retained the intron.

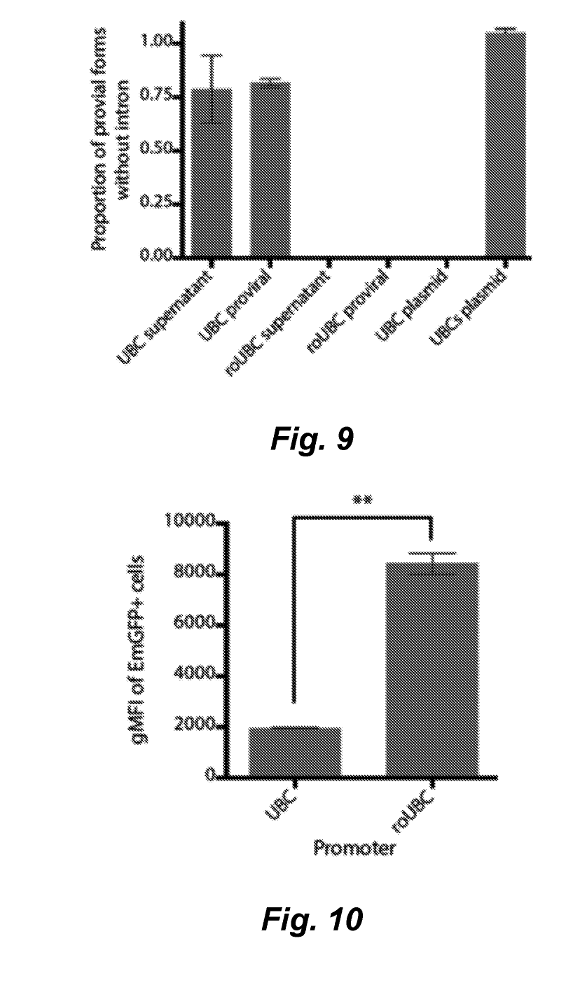

In order to assess whether events during transduction and reverse transcription influenced the proportion of proviral forms containing introns, we collected RNA from UBC viral supernatants and quantified the fraction of RNA genomes containing spliced UBC introns. We then compared this to the fraction of vector proviral forms containing spliced introns in K562 cells transduced with the same supernatants. These values agreed very closely, suggesting that the introns were already missing in vector particles and were therefore removed in the packaging cells (FIG. 9).

Loss of Intron Lowers Expression from UBC Promoter

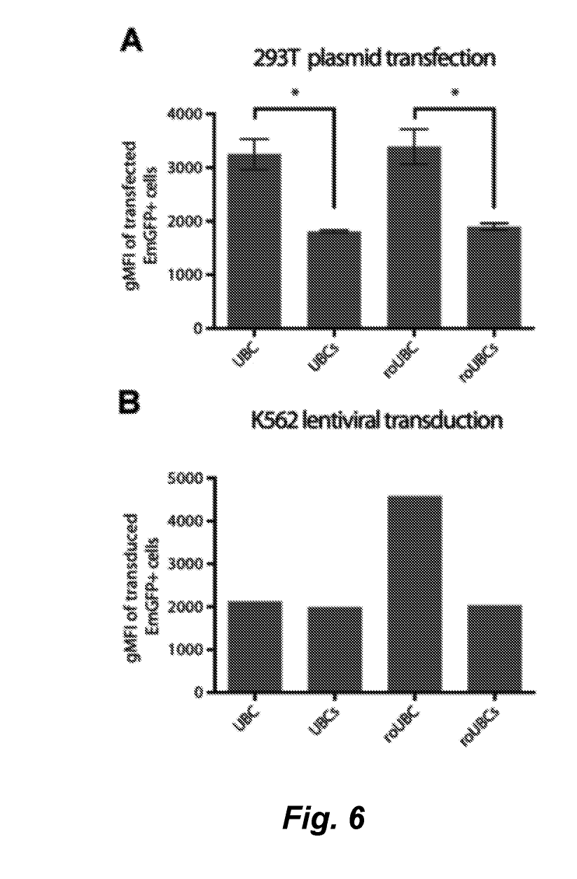

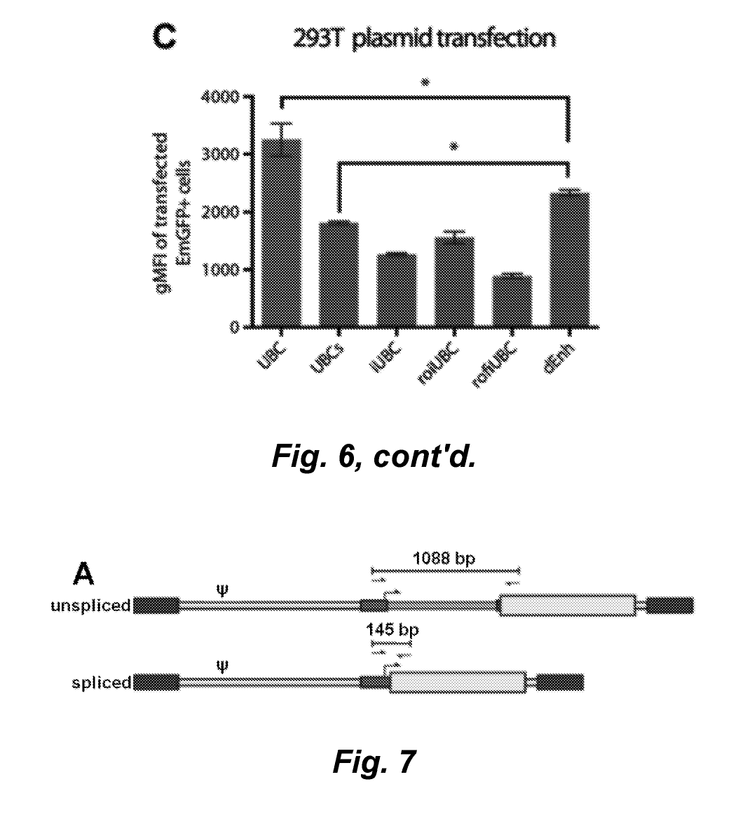

To assess the effect of intron loss on transgene expression, pCafe expression plasmids containing the full UBC promoter element or the truncated UBCs promoter with the intron region deleted were transiently transfected into 293T cells and analyzed at 48 h post-transfection via flow cytometry. The UBC promoter yielded significantly higher expression than the UBCs promoter, by a margin of ˜2-fold (FIG. 6, panel A). A similar 2-fold difference was observed between the roUBC and roUBCs constructs. Because these plasmids were transfected directly into cells, no intron loss was possible, and the UBC promoter plasmids tested all contained the intron.

Having established that the presence of the intron confers higher expression in these transfection experiments where intron loss was not possible, we next examined expression from the various constructs packaged as LVs 2 weeks after transduction of K562 cells. As the genetic analysis revealed that the majority of UBC LV forms lack the intron, we reasoned that the UBC vector would express levels of EmGFP similar to the UBCs vector. Indeed, the fluorescence of EmGFP-expressing cells in populations transduced with the UBC vector was nearly equivalent to that in populations transduced with UBCs vector (FIG. 6, panel B). In contrast, the roUBC vector showed ˜2-fold higher fluorescence in cells than the UBC vector, consistent with the genetic analysis indicating that the roUBC vector retains the intron.

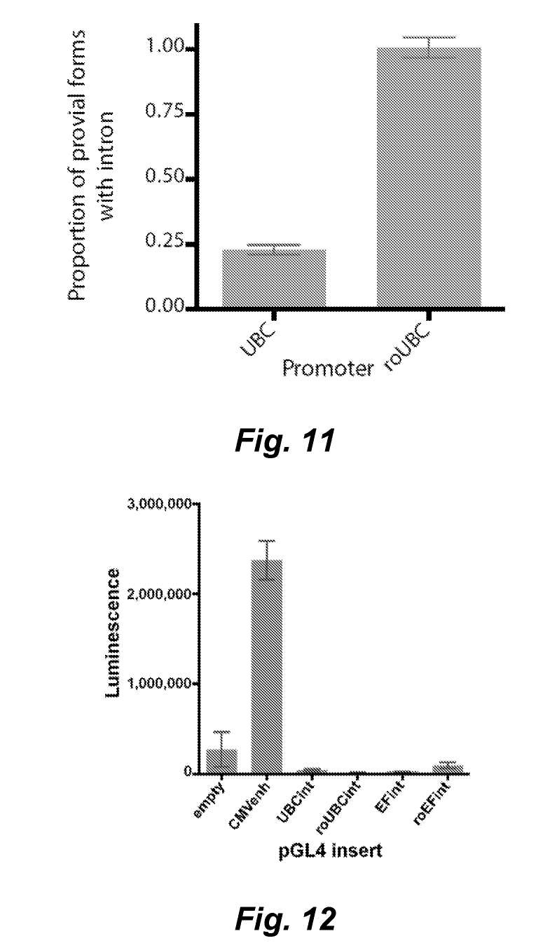

We also transduced human CD34+ hematopoietic stem and progenitor cells enriched from the peripheral blood of a healthy donor treated with granulocyte-colony stimulating factor to determine if the improved expression from the roUBC vector compared to the UBC vector would also be observed in a primary cell type relevant to lentiviral gene therapy. After 10 days of culture post-transduction in myeloid differentiation conditions, cells transduced with roUBC vector showed 4-fold higher expression than cells transduced with UBC (FIG. 10). Genetic analysis showed that intron loss was similar in the UBC-transduced cells to that observed in K562 cells and that the intron was fully maintained in roUBC-transduced cells (FIG. 11).

Positive Effect of UBC Intron on Expression is not Through Classical Enhancer Activity

Aside from reversal of the expression cassette, we also sought other ways to retain full expression of the UBC promoter fragment in an LV. We first investigated whether movement of the reported intronic enhancer sequence to a site immediately upstream of the promoter would lead to equivalent expression compared to the full-length UBC promoter fragment (Bianchi et al. (2009) Gene, 448: 88-101). Importantly, this variant lacked the intronic splice sites, which should allow its transmission in LVs. However, the resulting iUBC construct performed worse than UBCs (FIG. 6, panel C). roiUBC and rofiUBC were created and analyzed to assess whether the orientation of the enhancer sequence relative to the promoter was important, but these promoter variants expressed no better than iUBC (FIG. 6, panel C). We finally constructed dEnhUBC, in which the putative enhancer sequence was deleted, but the splicing sites were retained. This variant expressed slightly more EmGFP than UBCs, presumably due to improved nuclear export from splicing, but significantly less than UBC (FIG. 6, panel C). These results are consistent with a follow-up study on the UBC promoter fragment intron, which found that its enhancer activity was fully dependent on its position within the intron (Bianchi et al. (2013) PLoS ONE, 8: e65932). This behavior, termed intron-mediated enhancement, is poorly understood.

We reasoned that if the UBC intron sequence were indeed not a classical enhancer, then it should not increase expression from a heterologous minimal promoter. Indeed, when the intron sequence was placed in a luciferase reporter plasmid upstream of a minimal promoter in a forward or reverse orientation, no increase in luciferase expression over background was observed, in contrast to a plasmid in which a CMV enhancer sequence was placed upstream (FIG. 12). In fact, expression from these plasmids was significantly lower than from plasmids with the minimal promoter alone, consistent with the UBC intron sequence being repressive when placed outside the transcription unit. This repressive effect mirrors the reduction in expression seen when intronic sequences were placed upstream of the UBCs promoter form (FIG. 6, panel C). Interestingly, the same was true for EEF1A1 intron 1 in forward or reverse orientation (FIG. 12).

EEF1A1 Intron is Maintained in Proviral Forms and Aids in Maximal Expression

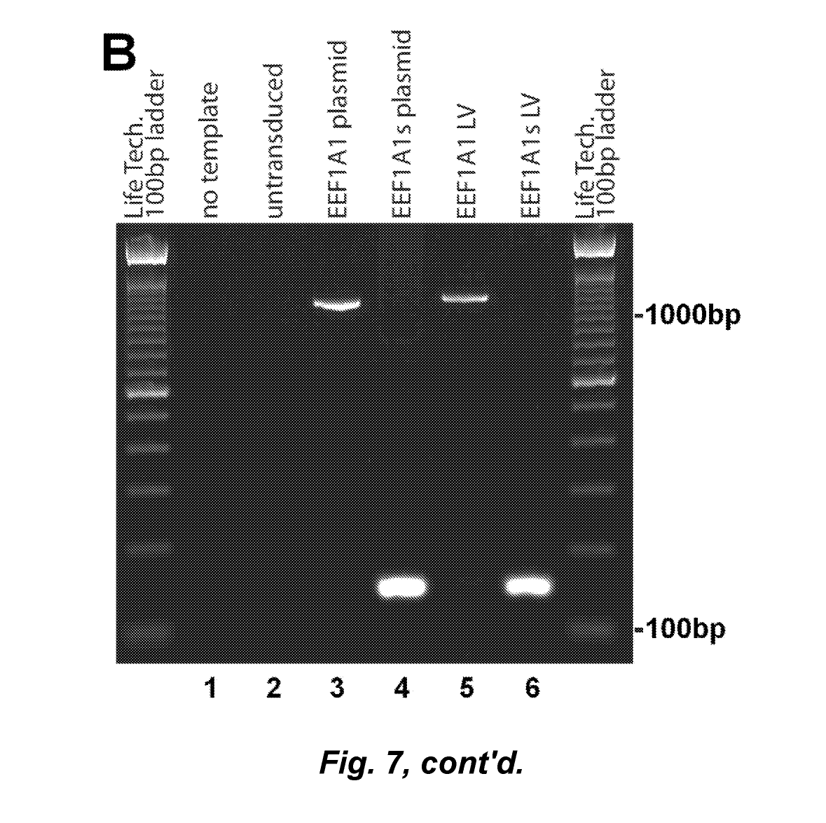

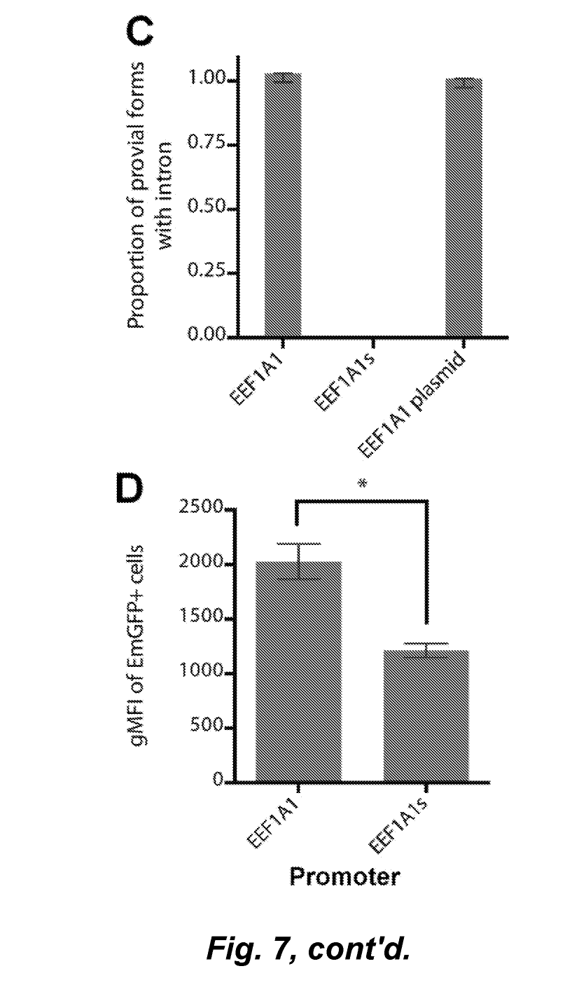

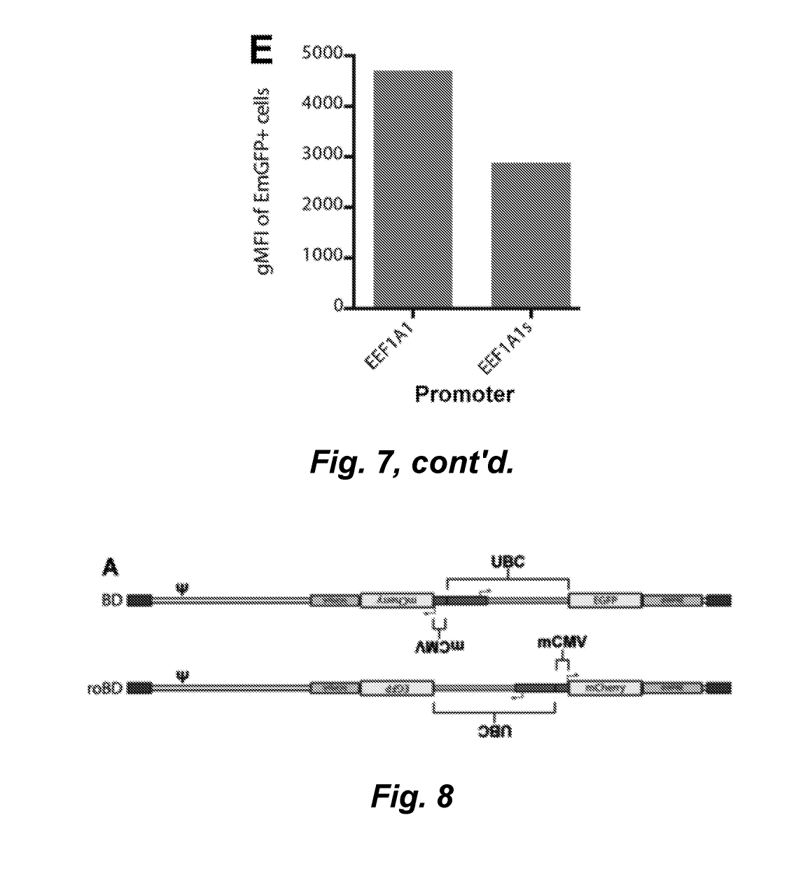

Because the observation of intron loss from the UBC promoter contrasts so starkly with reports on the elongation factor 1 alpha (EEF1A1) promoter fragment in LVs, we created expression vectors for transient transfection and lentiviral production with the EEF1A1 promoter fragment and an EmGFP reporter. PCR and ddPCR analysis of gDNA from transduced cells showed that nearly all vector forms retained the intron within the promoter (FIG. 7, panel B, lane 5). Extreme contrast adjustment of the gel electrophoresis image can reveal a barely detectable amount of short product at the length expected upon intron loss, but quantitative ddPCR analysis does not detect this small population of intron-lacking proviral forms (FIG. 7, panel C). Consistent with these observations and with a previous report (2), an ˜2-fold difference in expression between the intron-containing and intron-lacking promoters was observed both in transient transfection (FIG. 7, panel D) and transduction (FIG. 7, panel E) experiments, suggesting that the EEF1A1 promoter element's intron is indeed being faithfully transmitted in almost all cases.

Difference in Intron Transmission is Determined by Promoter Exon Sequences

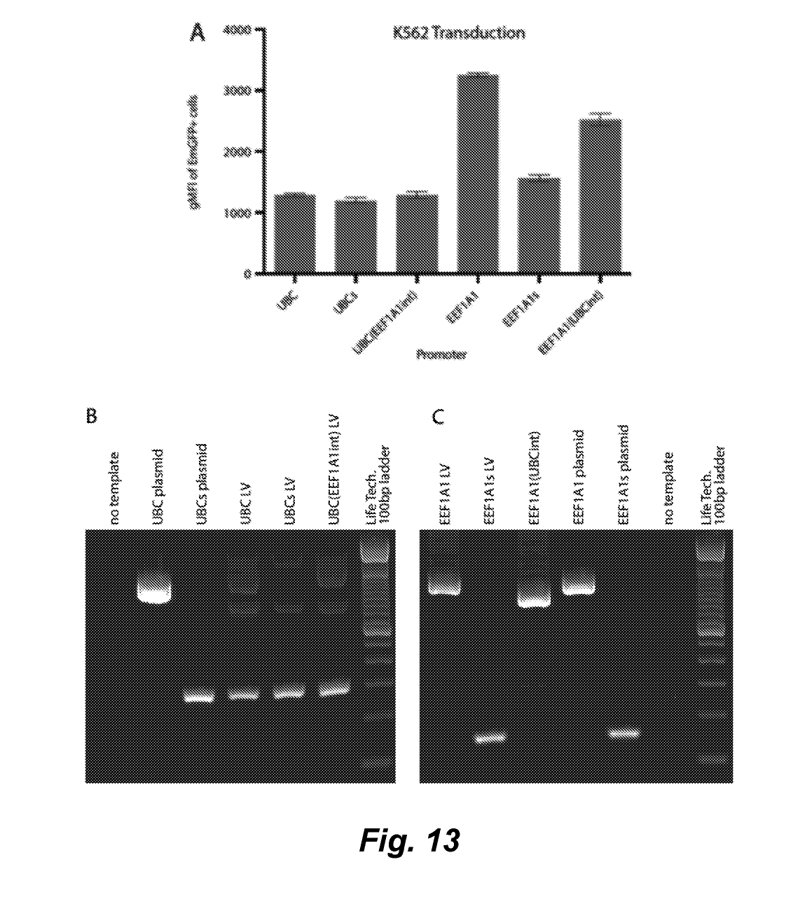

We hypothesized that the difference in intron retention between the UBC and EEF1A1 promoters was due to sequence determinants of splicing efficiency or splicing kinetics within the introns. To test this, we swapped the introns from one promoter to the other, creating UBC (EEF1A1int) and EEF1A1(UBCint) vectors. Surprisingly, we found that the UBC (EEF1A1int) LV lost the EEF1A1 intron and expressed similar levels of EmGFP to the intronless UBCs vector, while the EEF1A1(UBCint) maintained the UBC intron and expressed significantly more EmGFP than the intronless EEF1A1s vector (FIG. 13). These results suggest that the distinct exon sequences of the two promoters are determining whether the introns are retained during lentiviral production.

Expression Cassette Modification Maximizes Expression from UBC Bidirectional Vectors

We finally sought to improve expression from UBC promoter-based bidirectional vectors mediating coordinated expression of two transgenes in LVs (Kamata et al. PLoS ONE, 5: e11834; Amendola et al. (2005) Nat. Biotechnol., 23: 108-116). Because the vector design calls for a sense-strand orientation of the UBC promoter, we reasoned that the majority of proviral forms would lose the UBC intron and that reversal of the dual, divergent UBC and minimal cytomegalovirus (CMV) promoters would lead to increased expression due to intron inclusion with the UBC-promoted transgene (FIG. 8, panel A).

Genetic analysis of stably transduced 293T cells revealed that the UBC intron was lost 75% of the time from BD vectors, in which the UBC promoter is on the vector sense strand, whereas in roBD vectors, nearly all of the proviral forms contained the UBC intron (FIG. 8, panel B). This led to an increase in EGFP expression driven by the UBC promoter in stably transduced cells (FIG. 8, panel C). Surprisingly, in light of the expression data suggesting that the intron does not contain a traditional enhancer, mCherry expression driven by the minimal CMV promoter was also increased in retained UBC intron in roBD-transduced cells.

### Discussion

Lentiviral gene transfer has recently advanced into clinical gene therapy trials, with multiple successes and no clinically significant adverse events, and has also shown promise in many pre-clinical studies that will soon move into the clinic (Cartier et al. (2009) Science, 326(5954): 818-823; Cavazzana-Calvo et al. (2010) Nature, 467: 318-322; Aiuti et al. (2013) Science, 341(6148): 1233151; Biffi et al. (2013) Science, 341(6148): 1233158; Romero et al. (2013) J. Clin. Invest. 123(8): 3317-3330; Carbonaro et al. (2014) Mol. Ther., 22: 607-622). As therapies are developed for additional disorders, new vectors will be created bearing various genomic fragments for transgene regulation. Past promoter/transgene combinations have required the presence of introns for full activity and regulation, and it is likely that some future designs will require them as well.

Our results suggest that introns differ in terms of their likelihood of loss during vector production and transduction. While the human UBC promoter fragment was missing its intron in most proviral forms, the human EEF1A1 promoter fragment was not similarly affected. Inclusion of the UBC intron requires that the transgene cassette be reversed to avoid the processing of splicing machinery, but the EEF1A1 intron is maintained in almost every proviral form even though it is theoretically exposed to the spliceosome. A previous study indicates that the hybrid CAG promoter is also maintained throughout vector production and transduction (Ramezani et al. (2000) Mol. Ther., 2: 458-469). An intronless version of the EEF1A1 promoter has moved into clinical trials for both adenosine-deaminase-deficient severe combined immunodeficiency (ADA-SCID) and X-linked SCID (SCID-X1), and preclinical studies suggested that it will drive sufficient transgene expression for therapeutic effect. Our data indicate that a full EEF1A1 promoter containing intron 1 leads to roughly 2-fold higher transgene expression, an increase that could be necessary or beneficial for future vector designs. We also found that almost no proviral forms resulting from transduction with this vector lost their introns. Overall, these results illustrate the importance of full genetic characterization of retroviral vectors, as known or unknown introns can lead to transduced cells bearing highly variant vector forms. In the area of gene therapy, where product characterization is important from a regulatory standpoint, this variation is unlikely to find acceptance.

It has been reported that antisense RNA targeted to splice donor or acceptor sites can prevent splicing of primary transcripts (Morcos (2007) Biochem. Biophys. Res. Comm. 358:521-527). We therefore attempted to inhibit splicing of UBC vector genomes using U6-driven plasmids expressing 50 nt anti sense sequences to either the splice donor or splice acceptor site during lentiviral production. Unfortunately, these constructs did not lead to higher expression from UBC vectors upon transduction when used alone or in combination (data not shown). It is possible that this strategy could lead to retention of other introns in LVs, but it was ineffective for the UBC intron in our experiments. We found that the UBC promoter intron does indeed increase expression, as previously reported, but that the enhancer-like activity within the intron sequence is dependent on its placement inside the intron. This could be paralleled by future vector designs incorporating transgenes with endogenous introns for full activity, in which regulatory activity contained by intronic sequences might similarly not be mobile. Further research is also warranted to investigate why the UBC intronic sequences have a positive effect on expression when present within the transcription unit, but a negative effect when placed upstream of the promoter. This would likely have important implications for both endogenous gene regulation and transgene regulation for gene therapy and genetic engineering. Importantly, our data suggest that such introns are relatively safe payloads for integrating vectors, as they probably will not transactivate nearby promoters in the manner that has caused adverse events and subclinical clonal expansion in clinical gene therapy trials (Cavazzana-Calvo et al. (2010) Nature, 467: 318-322; Hacein-Bey Abina et al. (2003) Science, 302: 415-419; Hacein-Bey-Abina et al. (2008) J. Clin. Invest., 118: 3132-3142; Howe et al. (2008) J. Clin. Invest., 118: 3143-3150; Stein et al. (2010) Nat. Med., 16: 198-204).

The UBC intron and EEF1A1 intron 1 do not differ noticeably at the sequence level in terms of their adherence to canonical splice donor, acceptor and branch point sites, and our data from vectors in which the introns are swapped indicate that the sequence determinants of intron loss are not within the introns themselves but within the exonic sequences of the UBC and EEF1A1 promoters. This is unfortunate if true generally, as potential modifications to vectors to alter splicing would be limited dramatically in the majority of exons that are coding sequences. Biologically speaking, it is unsurprising, as exons in the human genome are known to contain exonic splicing enhancers as well as exonic splicing suppressors/silencers. These sequences control the efficiency of splicing of human introns, most of which are thought to be suboptimally defined (Zheng (2004) J. Biomed. Sci., 11: 278-294).

We believe that the difference in frequency of loss between these introns is linked to the speed at which they are spliced, which can be largely determined by exonic sequences. Future experiments could assess the splicing kinetics of these two genetic elements, the speed of which would be predicted to correlate inversely with intron transmission. While new work has examined the kinetics of transcript splicing and release from chromatin, the sequence determinants of the range of rates observed for different transcripts are not yet understood (Pandya-Jones et al. (2013) RNA, 19: 811-827). A better understanding of the determinants of splicing kinetics could direct the modification of the UBC promoter fragment to decrease splicing speed sufficiently to get intron-containing genomic RNA into vector particles, while maintaining efficient splicing during transgene expression.

### Example 2

### Enrichment of Human Hematopoietic Stem/Progenitor Cells Facilitates Transduction for Stem Cell Gene Therapy

Autologous hematopoietic stem cell (HSC) gene therapy for sickle cell disease has the potential to treat this illness without the major immunological complications associated with allogeneic transplantation. However, transduction efficiency by b-globin lentiviral vectors using CD34− enriched cell populations is suboptimal and large vector production batches may be needed for clinical trials. Transducing a cell population more enriched for HSC could greatly reduce vector needs and, potentially, increase transduction efficiency. CD34⁺/CD38⁻ cells, comprising ˜1%-3% of all CD34⁺ cells, were isolated from healthy cord blood CD34⁺ cells by fluorescence-activated cell sorting and transduced with a lentiviral vector expressing an anti-sickling form of betaglobin (CCL-β^{AS3}-FB). Isolated CD34⁺/CD38⁻ cells were able to generate progeny over an extended period of long-term culture (LTC) compared to the CD34⁺ cells and required up to 40-fold less vector for transduction compared to bulk CD34⁺ preparations containing an equivalent number of CD34⁺/CD38⁻ cells. Transduction of isolated CD34⁺/CD38⁻ cells was comparable to CD34⁺ cells measured by quantitative PCR at day 14 with reduced vector needs, and average vector copy/cell remained higher over time for LTC initiated from CD34⁺/38⁻ cells. Following in vitro erythroid differentiation, HBBAS3 mRNA expression was similar in cultures derived from CD34⁺/CD38⁻ cells or unfractionated CD34⁺ cells. In vivo studies showed equivalent engraftment of transduced CD34⁺/CD38⁻ cells when transplanted in competition with 100-fold more CD34⁺/CD38⁺ cells. This work provides evidence for the beneficial effects from isolating human CD34⁺/CD38⁻ cells to use significantly less vector and potentially improve transduction for HSC gene therapy.

### Introduction

Hematopoietic stem cell-based therapies can potentially treat a number of inherited and acquired blood cell diseases and exciting clinical progress has been made in recent years (Kohn et al. (2013) Biol. Blood Marrow Transplant. 19(suppl 1): S64-S69). Sickle cell disease (SCD) is a multisystem disease associated with severe acute illnesses and progressive organ damage leading to significant morbidity and early mortality (Platt et al. (1994) N. Engl. J. Med. 330: 1639-1644). It is one of the most common genetic disorders worldwide, affecting approximately 90,000 people in the U.S. Current treatments consist mainly of symptomatic therapy of anemia and pain. Hydroxyurea (HU) is another treatment option that induces fetal hemoglobin (HbF) production to inhibit polymerization of sickle hemoglobin (HbS) under low oxygen tension conditions; however, HU is not widely used for various reasons (Green and Barral (2014) Pediatr. Res. 75: 196-204; Brandow et al. (2013) Am. J. Hematol. 85: 611-613). The only potential cure of SCD is allogeneic hematopoietic stem cell transplant (HSCT). This typically requires a well-matched donor and may be accompanied by the need for long-term immune suppression with the possibility of graft rejection or graft versus host disease, although recent reports of effective reduced intensity condition in adult recipients of matched sibling stem cells holds promise (Hsieh et al. (2014) JAMA 312: 48-56).

Gene therapy with autologous hematopoietic stem cells (HSCs) is a promising treatment for SCD, potentially without the major immunological complications seen with allogeneic HSCT (Chandrakasan et al. (2014) Hematol. Oncol. Clin. North Am. 28: 199-216). The antisickling βAS3-globin gene when added to mouse and human hematopoietic stem/progenitor cells (HSPC) has been shown to have similar activity as HbF to inhibit red blood cell (RBC) sickling and prevent the manifestations of SCD (Levasseur et al. (2003) Blood 102: 4312-4319; Levasseur et al. (2004) J. Biol. Chem. 279: 27518-27524; Romero et al. (2013) J. Clin. Invest. 123: 3317-3330). However, lentiviral vectors carrying complex and relatively large human β-globin genomic expression cassettes have low titers; transduction of human CD34⁺ HSPC is only moderately effective and requires a relatively large amount of vector to be used, while yielding relatively low gene transfer (e.g., average vector copies per cell of 0.5-1.0). Unconcentrated production batches yield titers of <10⁶ transducing units (TU)/ml, necessitating large volumes of vector to be produced to perform transductions at clinical scale. Ideally, identifying ways to use less viral vector would avoid high vector production costs and allow treatment of more patients (Logan et al. (2004) Hum. Gene Ther. 15: 976-988).

CD38 is a type II membrane surface glycoprotein expressed on a variety of mature hematopoietic cells. CD38 expression is either low or absent on early HSPC populations (Hao et al. (1995) Blood, 86: 3745-3753; Hao et al. (1996) Blood, 88: 3306-3313; Albeniz et al. (2012) Oncol. Lett. 3: 55-60) and most definitions of the primitive, pluripotent human HSCs are contained within the CD34⁺/CD38⁻ fraction (Notta et al. (2011) Science, 333: 218-221). CD34⁺/CD38⁻ cells comprise only ˜1%-3% of CD34⁺ cells and thus are 50-100 times more enriched for HSPC than the unfractionated CD34⁺ population. CD34⁺/CD38⁻ cells have the capacity for long-term proliferation and blood cell production exceeding that of unfractionated CD34⁺ cells (Hao et al. (1995) Blood, 86: 3745-3753; Hao et al. (1996) Blood, 88: 3306-3313). Previously published studies have demonstrated the ability to transduce primary human BM CD34⁺ cells with the CCL-β^{AS3}-FB LV vector (Romero et al. (2013) J. Clin. Invest. 123: 3317-3330) with moderate efficiency using relatively high vector concentrations. We postulated that further purification of HSPC beyond the standard CD34⁺ cell-enriched fractions by isolating CD34⁺/CD38⁻ cells would reduce the absolute number of target cells to be treated ex vivo and significantly less vector would be needed per treated subject.

Here, we show that human cord blood (CB) CD34⁺/CD38⁻ cells isolated using fluorescence-activated cell sorting (FACS) could be transduced with up to 40-fold less viral vector and still achieve a vector copy number (VCN) comparable to or higher than that seen in the unfractionated CD34⁺ cell population. These results demonstrate the potential for using CD34⁺/CD38⁻-enriched HSPC to improve transduction of HSC for increased efficacy in gene therapy of SCD.

### Materials and Methods.

Vectors

Construction of the CCL-β^{AS3}-FB and CCL-MND-GFP has been described (Romero et al. (2013) J. Clin. Invest. 123: 3317-3330). CCL-Ubiq-mCitrine-PRE-FB-2XUSE, CCL-UbiqmStrawberry-PRE-FB-2XUSE, and CCL-Ubiq-mCerulean-PRE-FB-2XUSE were constructed using the CCL vector backbone (Zufferey et al. (1998) J Virol. 72: 9873-9880), a human ubiquitin promoter (Lois et al. (2002) Science, 295: 868-872), the fluorescent genes purchased from Addgene (Cambridge, Mass.), an optimized post-transcriptional regulatory element (PRE; (Zanta-Boussif et al. (2009) Gene Ther. 16: 605-619; Schambach et al. (2006) Gene Ther. 13: 641-645)), and two tandem copies of the SV40 polyadenylation enhancer sequences USE (Schambach et al. (2007) Mol. Ther. 15: 1167-1173). Lentiviral vectors were packaged with a VSV-G pseudotype and concentrated and titered as described (Cooper et al. (2011) J. Virol. Meth. 177: 1-9). CCL-β^{AS3}-FB was also packaged with an RD-114 pseudotype using the RD114/TR plasmid (Sandrin et al. (2002) Blood 100: 823-832; Bell et al. (2010) Exp. Biol. Med. 234: 1269-1276; Rasko et al. (1999) Proc. Natl. Acad. Sci. USA, 96: 2129-2134) and titered as described (Cooper et al. (2011) J. Virol. Meth. 177: 1-9). Different preparations of the VSV-G pseudotyped CCL-β^{AS3}-FB had titers of 6×10⁸-6×10⁹ TU/ml after 300-1,000× concentration, compared to 2.7×10⁷ TU/ml for the concentrated RD114/TR pseudotype preparation.

Sample Collection

Umbilical CB was obtained after vaginal and cesarean deliveries at UCLA Medical Center (Los Angeles) after clamping and cutting of the cord by drainage of blood from the placenta into sterile collection tubes containing the anticoagulant citrate-phosphate-dextrose. All CB specimens were obtained according to guidelines approved by the University of California, and have been deemed as anonymous medical waste exempt from IRB review. Cells were processed within 48 hours of collection. Mononuclear cells (MNCs) were isolated from CB using Ficoll Hypaque (Stem Cell Technologies, Vancouver, BC, Canada) density centrifugation. Immunomagnetic column separation was then used to enrich for CD34⁺ cells by incubating the MNCs with anti-CD34 microbeads (Miltenyi Biotec, Inc., Bergisch Gladbach, Germany) at 4° C. for 30 minutes. The cells were then sent through the immunomagnetic column and CD34⁺ cells collected. CD34⁺ cells were placed in cryovials with freezing medium (10% dimethyl sulfoxide [Sigma Aldrich, St. Louis, Mo.], 90% FBS) and cryopreserved in liquid nitrogen until needed.

Fluorescent Antibody Labeling and CD34⁺/CD38⁻ Cell Sorting

The CD34⁺ cells were thawed, washed, and resuspended in 75 ml of phosphate-buffered saline (PBS) for incubation with fluorescent-labeled antibodies. Undiluted phycoerythrin (PE) conjugated anti-CD38 (20 ml) and undiluted allophycocyanin (APC) conjugated anti-CD34 (5 ml) (all antibodies from BD Sciences, San Jose, Calif.) were added and the cells were incubated for 30 minutes at 4° C. in the dark. After incubation, cells were washed once in PBS. FACS was performed on a FACS Aria II (BD Biosciences).

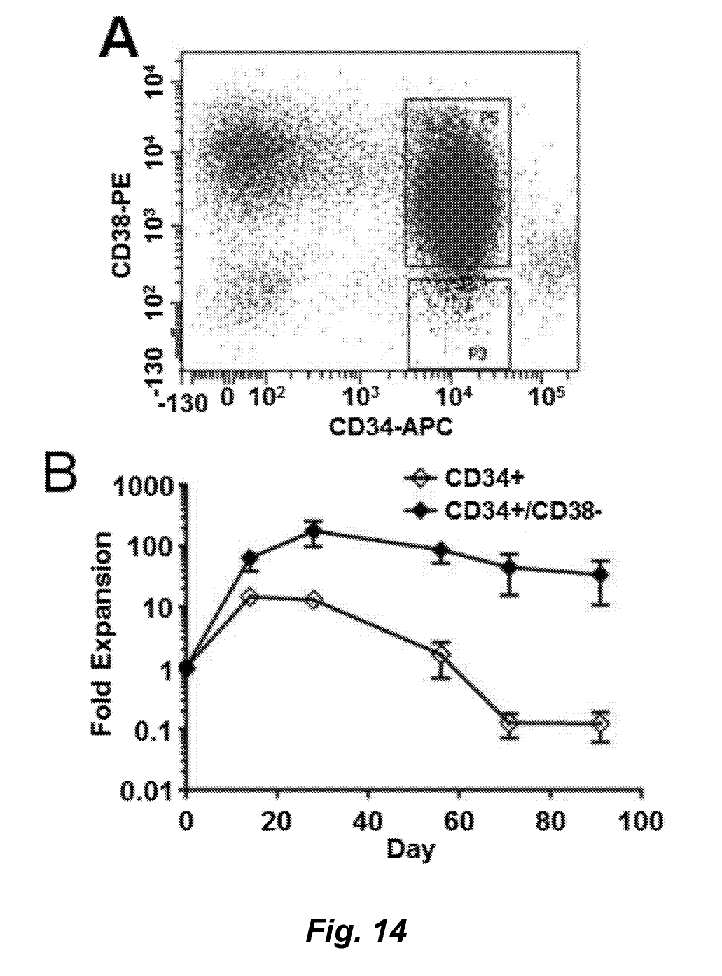

The viable MNC population was gated by forward scatter and 4′,6-diamidino-2-phenylindole (Life Technologies, Grand Island, N.Y.) staining. The gated region was used to define the CD34⁺ cell population (FIG. 14, panel A). P3 was used to define the F1 CD34⁺/38⁻ cell population, which was 2.5% of the APC positive cells that were negative for PE. P5 was used to define the CD34⁺/CD38⁺ cells that were positive for APC and positive for PE. These gating strategies were used for all sorting experiments.

Lentiviral Vector Transduction

After cell sorting, CD34⁺ and CD34⁺/CD38⁻ cells were placed in individual wells of a nontissue culture treated plate coated with retronectin (20 mg/ml retronectin, Takara Shuzo, Co., Japan) at a cell density of 6.3×10⁴-7.5×10⁵ cells per milliliter. Prestimulation was performed for 18-24 hours at 37° C., 5% CO₂ in Transduction Medium (serum free X-vivo 15 medium (Lonza, Basel, Switzerland) containing 1×L-glutamine/penicillin/streptomycin (L-Glut/Pen/Strep) (Gemini Bio-Products, West Sacramento, Calif.), 50 ng/ml human stem cell factor (hSCF) (StemGent, Cambridge, Mass.), 20 ng/ml human interleukin-3 (hIL-3) (R&D Systems, Minneapolis, Minn.), 50 ng/ml human thrombopoietin (R&D Systems), and 50 ng/ml human Flt-3 ligand (Flt-3) (PeproTech, Rocky Hill, N.J.)). After prestimulation, the desired viral vector (CCL-β^{AS3}-FB, CCLMND-GFP, mStrawberry, mCerulean, mCitrine or β^{AS3}-FB-RD114) was added to each well at the specified vector concentration (typically 2×10⁷ TU/ml unless otherwise specified) and again incubated at 37° C., 5% CO₂ for 24 hours. The cells were then washed and transferred to a tissue cultured treated plate for myeloid differentiation in basal bone marrow medium (Iscove's Modified Dulbecco's Medium (IMDM) (Life Technologies, Grand Island, N.Y.), 1× L-Glut/Pen/Strep, 20% FBS, 0.52% bovine serum albumin (BSA)) with 5 ng/ml IL-3, 10 ng/ml IL-6, and 25 ng/ml hSCF at 37° C., 5% CO₂. Fresh medium was added as needed over a 14-day period. After 14 days in culture, VCN was determined by qPCR or digital droplet PCR (ddPCR) (Hindson et al. (2011) Anal. Chem. 15: 8604-8610) and fluorescent reporter gene expression was analyzed using flow cytometry.

Long-Term Stromal Cultures and Methylcellulose Cultures

Two days prior to planned CD34⁺ cell sorting, MS5 murine stromal cells (Suzuki et al. (1991) Leukemia, 6: 452-458) were thawed, irradiated at 10,000 cGy, and then plated (3×10⁴ cells/well) in 96-well plates in stromal medium (IMDM [Life Technologies Grand Island, N.Y.], FBS 10%, 2-mercaptoethanol) to form pre-established stromal layers for the long-term cultures (LTCs). Sorted CD34⁺ and CD34⁺/CD38⁻ cells were cocultured on the irradiated stroma in LTC medium (IMDM, 30% FBS, 10% BSA, 2-mercaptoethanol, 10⁶ mol/l hydrocortisone, 1× L-Glut/Pen/Strep, along with 10 ng/ml interleukin-3 [IL-3], 50 U/ml IL-6, and 50 ng/ml human stem cell factor (hSCF)) (Hao et al. (1996) Blood, 88: 3306-3313: Breems et al. (1998) Blood, 91: 111-117; Koller et al. (1995) Blood, 86: 1784-1793; Bennaceur-Giscelli et al. (2001) Blood, 97: 435-441). At 2-4 week intervals, samples of nonadherent cells were removed from cultures. Their numbers were determined by viable cell counting with a hemocytometer (Thermo Fisher Scientific, Pittsburgh, Pa.) and genomic DNA was extracted using Purelink Genomic DNA Mini kit (Invitrogen, Carlsbad, Calif.) for qPCR, as described below.

Differentiation and mRNA Expression Analysis

The in vitro erythroid differentiation assay is based on a protocol adapted from Douay and Giarratana (2009) Meth. Mol. Biol. 482: 127-140 as modified by Romero et al. (2013) J. Clin. Invest. 123: 3317-3330. After prestimulating and transducing the FACS isolated CD34⁺/CD38⁻ cells and unfractionated CD34⁺ cells, the cells were transferred to erythroid culture. The VCN was analyzed using qPCR of the HIV-1 packaging signal sequence Psi in the LV provirus and normalized to the human cellular autosomal gene syndecan 4 (SDC4) to calculate the VCN as described by Cooper et al. (2011) J Virol. Meth. 177: 1-9. HBβ^{AS3} mRNA expression was determined as previously described by Romero et al. (2013) J. Clin. Invest. 123: 3317-3330.

Transplantation of Transduced Human CB CD34⁺/CD38⁻ Cells in Immune-Deficient Mice

Unfractionated CD34⁺, CD34⁺/CD38⁺, and CD34⁺/CD38⁻ cells from healthy donor CB were transduced separately with lentiviral vectors carrying the different fluorescent marker genes all at 2 3×10⁷ TU/ml (MOI 5-140). Mock transduced (5×10⁵) and control unfractionated transduced CD34⁺ (5×10⁵) cells were individually transplanted by tail vein injection into 6-10 weeks old, immune-deficient NOD.Cg-Prkd^{scid}Il2rg^{tm1Wjil}/SzJ (NSG) mice (Jackson Laboratory, Sacramento, Calif.) after 250 cGy total body irradiation. CD34⁺/CD38⁻ and CD34⁺/CD38⁺ cells were mixed at a 1:99 ratio so that 2×10³ CD34⁺/CD38⁻ cells and 2×10⁵ CD34⁺/CD38⁺ cells were cotransplanted by tail vein injection into 6-10 weeks old NSG mice after irradiation.

After 8-12 weeks, the mice were euthanized and the BM was analyzed for engraftment of human cells by flow cytometry using APC-conjugated anti-human CD45 versus Horizon V450-conjugated anti-murine CD45 (BD Biosciences). After antibody incubation, RBCs were lysed using BD FACS-Lysing Solution (BD Biosciences). The percentage of engrafted human cells was defined as the % huCD45⁺/% huCD45⁺+% muCD45⁺ cells). From among the huCD45⁺ cells, expression of mCitrine, mStrawberry, and mCerulean was analyzed using flow cytometry.

The average VC/human cell was measured in the murine BM samples with positive engraftment of human cells using ddPCR (Table 4). Reaction mixtures were prepared consisting of 22 ml volumes containing 1× ddPCR Master Mix (Bio-Rad, Hercules, Calif.), primers, and probe specific to either the HIV-1 Psi region, to detect all vectors, or to each of the fluorescent reporter genes (400 nM and 100 nM for primers and probe, respectively), DraI (40 U; New England Biolabs, Ipswich, Mass.), and 1.1 ml (4 ml for cfu) of the genomic DNA sample. Droplet generation was performed as described in Hindson et al. (2011) Anal. Chem. 15: 8604-8610. Thermal cycling conditions consisted of 95° C. 10 minutes, 94° C. 30 seconds and 60° C. 1 minute (55 cycles), 98° C. 10 minutes (1 cycle), and 12° C. hold (Table 3). The specific amplified portions of the gene were normalized for the percentage of human cells present in the marrow collected from the NSG mice using primers to the autosomal human gene SDC4 gene to adjust for the presence of murine cells in the samples. All animals involved in experiments were cared for and handled in accordance with protocols approved by the UCLA Animal Research Committee under the Division of Laboratory Medicine.

TABLE 2
In vitro vector copy number of cells transplanted
into NOD.Cg-Prkd^{scid}Il2rg^{tmlWjiL}/SzJ mice.

| Experimental arm | In vitro Vector Copy Number | | |
|---|---|---|---|
| Transplant # | 1 | 2 | 3 |
| Nontransduced CD34+ | 0 | 0 | 0 |
| Transduced CD34+ | — | 1.58 | 2.81 |
| Transduced CD34+/CD38+ | 1.48 | 0.40 | 2.66 |
| Transduced CD34+/CD38− | 1.94 | 7.59 | 11.50 |

TABLE 3
PCR primers.

| | | SEQ |
|---|---|---|
| | | ID |
| Primer/Probe | Sequence | NO |
| SDC4 | | |
| forward primer | 5′-CAGGGTCTGGGAGCCAAGT-3′ | 10 |
| reverse primer | 5′-GCACAGTGCTGGACATTGACA-3′ | 11 |
| Probe | 5′ HEX-CCCACCGAA-ZEN- | 12 |
| | CCCAAGAAACTAGAGGAGAAT-IBFQ* 3′ | |
| Psi U5 | | |
| forward primer | 5′ AAGTAGTGTGTGCCCGTCTG 3′ | 13 |
| reverse primer | 5′ CCTCTGGTTTCCCTTTCGCT 3′ | 14 |
| Probe | 5′ FAM-ATCGTCGGC-ZEN- | 15 |
| | ATCAAGTTGGACATCACCT-IBFQ 3′ | |
| mCerulean | | |
| forward primer | 5′ GACCACCCTGACCTGG 3′ | 16 |
| reverse primer | 5′ CGCTCCTGGACGTAGCCTT 3′ | 17 |
| Probe | 5′ FAM-AGCACGACT-ZEN- | 18 |
| | TCTTCAAGTCCGCCAT-IBFQ 3′ | |
| mStrawberry | | |
| forward primer | 5′ TCAAGACCACCTACAAGGCCAAGA | 19 |
| reverse primer | 5′ ACAGTTCCACGATGGTGTAGTCCT 3′ | 20 |
| Probe | 5′ FAM-ATCGTCGGC-ZEN- | 21 |
| | ATCAAGTTGGACATCACCT-IBFQ 3′ | |
| mCitrine | | |
| forward primer | 5′ TTCGGCTACGGCCTGATCT 3′ | 22 |
| reverse primer | 5′ CGCTCCTGGACGTAGCCTT 3′ | 23 |
| Probe | 5′ FAM-AGCACGACT-ZEN- | 24 |
| | TCTTCAAGTCCGCCAT-IBFQ 3′ | |

*IBFQ = IOWA BLACK ®, ZEN-internal modification from IDT-integrated DNA technologies

Low Density Lipoprotein Receptor Expression Analysis

Cells were collected and sorted as previously described in the fluorescent antibody staining and cell sorting section. CD34⁺ and CD34⁺/CD38⁻ cells were placed into individual wells of a nontissue culture treated plate coated with retronectin (20 mg/ml retronectin, Takara Shuzo, Co., Japan) in Transduction Medium for at 37° C., 5% CO₂. At 24 hours and 48 hours in culture, the cells were harvested for analysis of low density lipoprotein (LDL) receptor expression, compared to the cells prior to culture (0 hours) using flow cytometry. The cells were washed and resuspended in 90 ml of PBS for incubation with fluorescent-labeled antibody, 10 ml undiluted APC conjugated anti-human LDL receptor (R&D Systems, Minneapolis, Minn.). The cells were incubated for 30 minutes at 4° C. in the dark. After incubation, cells were washed once in PBS and analyzed with the LSR Fortessa for analysis. APC-positive cells were considered to be positive for expression of the LDL receptor.

Statistical Analyses

Continuous outcome variables such as means and SEs by experimental conditions are presented in figures. Pairwise comparison was performed by either unpaired t-test within the framework of one-way or two-way ANOVA. Two group comparisons by Wilcoxon rank sum test was performed when the assumption of normality was not met. Mixed linear model was used to compare two groups over time. A p-value of 0.05 was used as the significance threshold.

### Results.

Isolation of CB CD34⁺/CD38⁻ Cells Using FACS

Healthy donor CB was enriched for CD34⁺ cells using immunomagnetic columns. Portions of the unfractionated CD34⁺ cells were sorted by flow cytometry to isolate CD34⁺/CD38⁻ cells, operationally defined as cells with the lowest 2.5% for CD38 expression (FIG. 14, panel A). Starting with 2.2-6.8×10⁶CD34⁺ cells isolated from CB units, 6×10³-6×10⁴ (mean=2.5×10⁴) CD34⁺/CD38⁻ cells were isolated, representing a range of 36%-99% of the theoretical yield (n=11).

When put into long-term culture, the unfractionated CD34⁺ cells expanded ˜10-fold over the first month, and then declined in numbers (FIG. 1B). LTCs initiated with CD34⁺/CD38⁻ cells expanded to a greater extent (˜100-fold) and maintained stable cell numbers for more than 3 months (FIG. 14, panel B), demonstrating the greater generative capacity of the more primitive CD34⁺/CD38⁻ populations, compared to the bulk CD34⁺ cells.

Assessment of Transduction of CB CD34⁺ Versus CD34⁺/CD38⁻ Cells

Transduction of CD34⁺ and CD34⁺/CD38⁻ cells from CB of healthy donors (n511) with the CCL-β^{AS3}-FB lentiviral vector was compared. Cell density and vector concentration, and hence multiplicity of infection (MOI), were kept constant for the two cell types within an experiment, using either equal numbers of CD34⁺ and CD34⁺/CD38⁻ cells in identical volumes or adjusting the total volume of the culture when different cells numbers were transduced. Transduced cells were either cultured for 2 weeks under short-term in vitro myeloid differentiation conditions, grown in methylcellulose colony forming unit (CFU) assay (14 days), or grown in long-term myeloid cultures (90 days) to compare colony-forming capabilities and VCN.

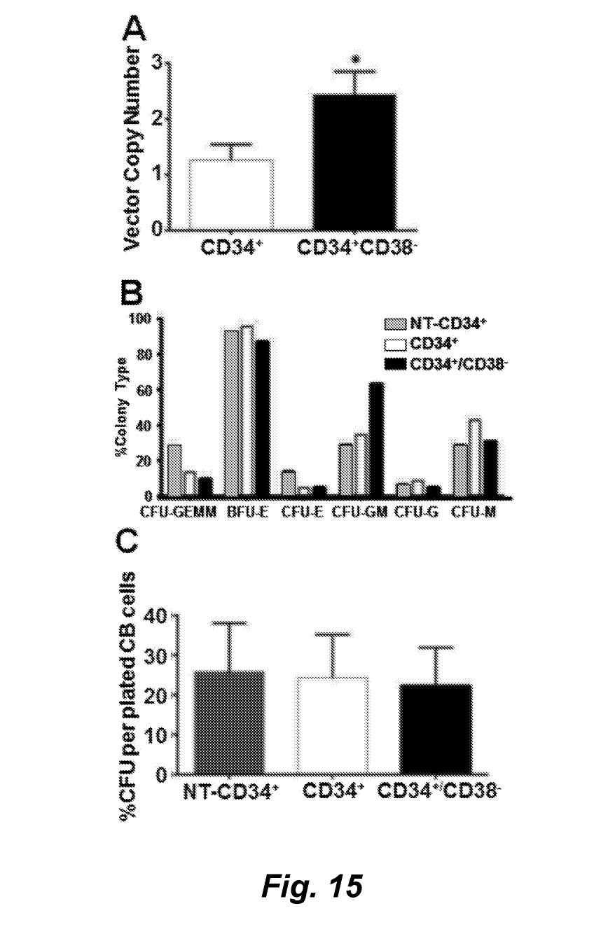

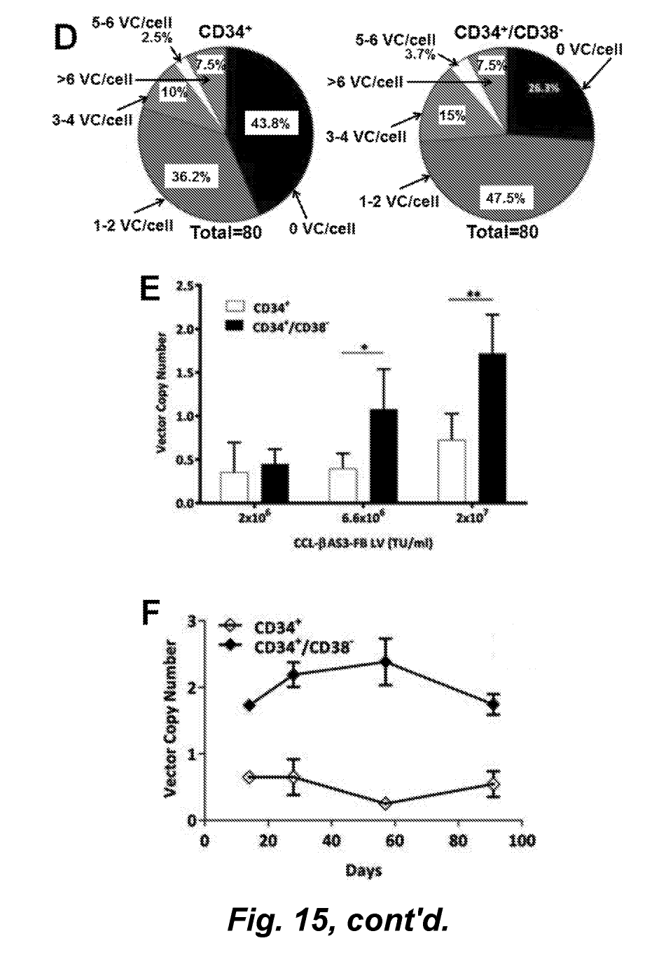

Genomic DNA isolated from cells was analyzed by quantitative (qPCR) for the HIV-1 psi region of the vector at day 14 to determine average vector copy number/cell (VCN). In each sample, transduction of the CD34⁺/CD38⁻ cells was equal to or greater than transduction of CD34⁺ cells (Table 2). The cells produced from the transduced CD34⁺/CD38⁻ cells had a significantly higher VCN of 2.43±0.41 compared to 1.25±0.28 from the transduced CD34⁺ cultures (n=11, p=0.02) (FIG. 15, panel A).

The types of colonies formed by CD34⁺ cells and CD34⁺/CD38⁻ cells were not different (FIG. 15, panel B). Colonies were formed by 25.7% of the nontransduced CD34⁺ (NT-CD34⁺), 24.3% of transduced CD34⁺, and 22.3% of transduced CD34⁺/38⁻ cells plated in methylcellulose (FIG. 15, panel C). qPCR of individual CFU to detect and quantify the CCL-β^{AS3}-FB vector sequences demonstrated that the percentage of transduced colony-forming progenitors from CD34⁺/CD38⁻ cells (73.8% with a mean VCN=2.12) was higher than from CB CD34⁺ cells (56.2% with a mean VCN=1.75) (n=80 colonies, each) (FIG. 15, panel D) (p=0.52). CFU formed from CD34⁺/38⁻ cells showed a larger percentage of colonies with 1-2 VC/cell (47.5%) compared to those formed from unfractionated CD34⁺ cells (36.2%) (FIG. 15, panel D).

Vector dose-response experiments were performed to examine the relative ability of the CCL-β^{AS3}-FB vector to transduce human CB CD34⁺ and CD34⁺/CD38⁻ cells, using a range of vector concentrations during transduction from 2×10⁶ to 2×10⁷ TU/ml. A dose-related increase in gene transfer (VCN measured by qPCR) at day 14 was seen with increasing vector concentrations in both cell populations (FIG. 15, panel E). However, at every vector concentration tested, the resultant VCN was higher for the CD34⁺/CD38⁻ cells (p=0.05 at 6.6×10⁶ TU/ml, p=0.002 at 2×10⁷ TU/ml) than for the CD34⁺ cells; thus considerably lower concentrations of viral vector (2×10⁶ TU/ml) could be used to transduce the CD34⁺/CD38⁻ cells and still match the level of transduction achieved with CD34⁺ cells at higher vector concentration (2×10⁷ TU/ml) (FIG. 15, panel E).

Transduced CB CD34⁺ and CD34⁺/CD38⁻ cells (n=3) were grown for 90 days in LTC on MS5 stromal cells, and cell samples were analyzed at several time points for VCN (FIG. 15, panel F). At each time point, there was a higher VCN in the cultures from CD34⁺/CD38⁻ cells (1.6-2.3) compared to cultures from the unfractionated CD34⁺ population (0.3-0.6) (p=0.0004), with statistically significant time trend difference (p=0.03).

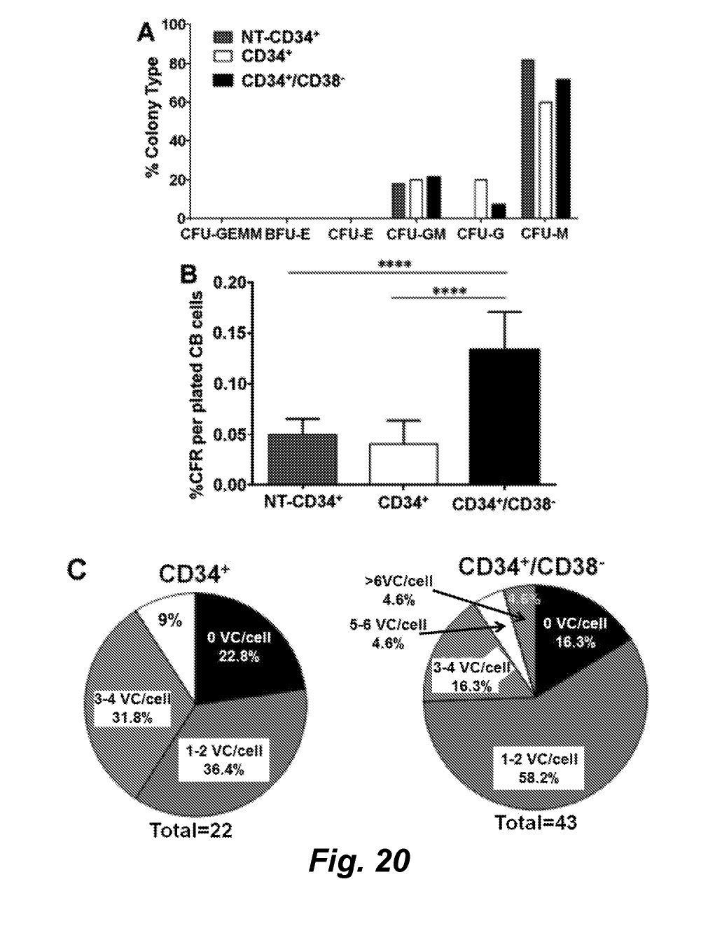

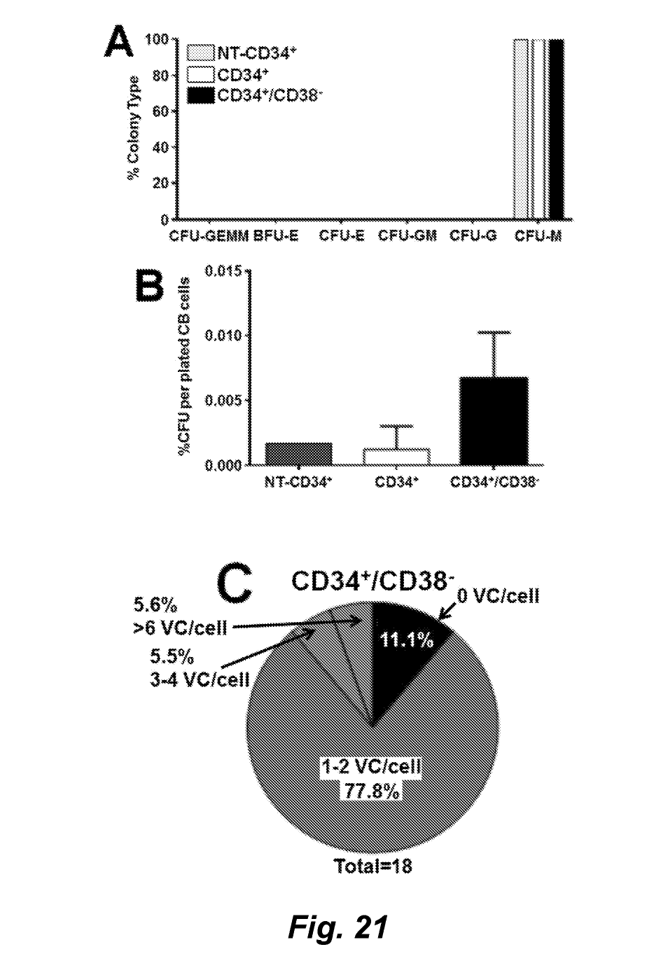

LTCs initiated from CD34⁺/CD38⁻ cells had increasingly higher frequencies of colony-forming cells compared to cultures initiated form CD34⁺ cells. At day 30 of the LTC, 0.05% of the cells from cultures of NT CD34⁺ cells, 0.04% of transduced CD34⁺, and 0.13% of transduced CD34⁺/38⁻ cells (p<0.0001) plated in methylcellulose produced colonies (FIG. 20). At day 60 of the LTC, colonies were produced by 0.0017% of the cells derived from NT CD34⁺ cells, 0.0025% from transduced CD34⁺, and 0.0067% from transduced CD34⁺/CD38⁻ cells plated in methylcellulose (FIG. 21).

Higher percentages of transduced colony-forming progenitors were also present in the cultures initiated from CD34⁺/CD38⁻ cells than from those initiated with CD34⁺ cells. When analyzed at day 30 of LTC by ddPCR, 83.7% (average VCN52.3) formed from CD34⁺/CD38⁻ cells were positive for the CCL-β^{AS3}-FB vector compared to 77.3% (average VCN=2.2) of individual CFU derived from cultures of unfractionated CD34⁺ (FIG. 20). At 60 days, the unfractionated CD34⁺ cells produced only three colonies, one of which had a VCN of 0.5 while the other two had a VCN of 0. Twenty-two CFU were formed from CD34⁺/CD38⁻ cells and 16 (88.8%) were positive for the CCL-β^{AS3}-FB vector (average VCN=1.52) (FIG. 21).

To determine whether the higher transduction of the CD34⁺/CD38⁻ cells would occur with vectors other than the CCL-β^{AS3}-FB vector, the transduction efficiency of CD34⁺ and CD34⁺/CD38⁻ cells by a high titer green fluorescent protein (GFP)-expressing LV vector (CCL-MND-GFP) was assessed in dose response experiments. Both cell populations were transduced at equal cell densities with the CCL-MND-GFP LV vector at the concentration of 2×10⁶, 6.6×10⁶, and 2×10⁷ TU/ml (MOI=4, 40, and 400, respectively). Again a dose-related increase in gene transfer (VCN measured by ddPCR) at day 14 was seen with increasing vector concentrations in both cell populations (FIG. 16, panel A). At vector concentrations of 6.6×10⁶ and 2×10⁷ TU/ml, the resultant VCN was higher for the CD34⁺/CD38⁻ cells (CD34⁺ mean VCN=2.25±0.15 vs. 1.36±0.05 at 6.6×10⁶ TU/ml and 4.32±0.6 vs. 3.37±0.07, p=0.02 at 2×10⁷ TU/ml) than for the CD34⁺ cells. On day 14, the percentages of GFP expressing cells were determined using flow cytometry (FIG. 16, panel B) and ranged from 45% to 81% in CD34⁺ cells compared to 45%-97% in CD34⁺/CD38⁻ cells (FIG. 16, panel C), which was not significantly different, p=0.29.

In Vitro Erythroid Differentiation of CB CD34⁺/CD38⁻ Cells

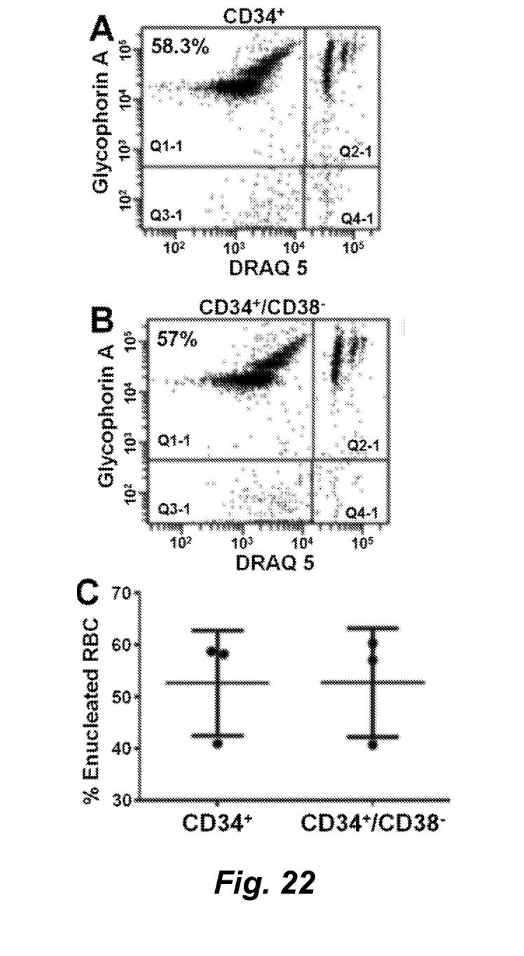

To compare β^{AS3}-globin expression by the CCL-β^{AS3}-FB vector after transduction of CD34⁺ and CD34⁺/CD38⁻ cells, transduced cells were put into an in vitro erythroid differentiation model (Douay and Giarratana (2009) Meth. Mol. Biol. 482: 127-140) to produce mature RBCs that support expression by the β-globin gene cassette. Vector expression was measured using quantitative reverse transcriptase polymerase chain reaction (qRT-PCR) to specifically quantify both the HBβ^{AS3} transcript from the vector and the total b-globin-like transcripts (endogenous HBB and HBβ^{AS3}). CD34⁺/CD38⁻ and CD34⁺ cells from healthy CB donors were transduced with the CCL-β^{AS3}-FB LV vector and control samples were mock transduced. After 24 hours, the cells were differentiated into erythroid cells for 27 days. Enucleated RBCs were identified at the end of the differentiation (day 27) by double staining with an antibody to the erythroid membrane glycoprotein GpA and the DNA labeling fluorescent dye, DRAQ5. Enucleated RBCs were defined as being GpA⁺/DRAQ5⁻ (FIG. 22). Final cell numbers, differentiation markers, and percentage enucleation were similar between the two cell populations, ranging from 52.6%±1.06% enucleated erythrocytes from the unfractionated CD34⁺ cells and 52.7%±1.08% from the CD34⁺/CD38⁻ cells (p=0.87).

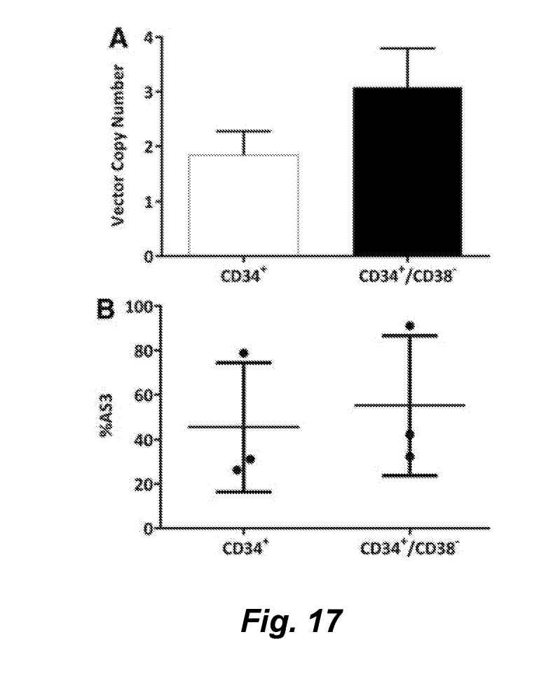

Higher VCN were present in the erythroid progeny of the CD34⁺/CD38⁻ cells, compared to the progeny of the CD34⁺ cells. At 2 weeks of culture, the cultures from the CD34⁺/CD38⁻ cells had an average VCN of 3.08±0.71 compared to an average VCN of 1.84±0.44 from the CD34⁺ cells (n=3, p=0.26) (FIG. 17, panel A).

Cells transduced with the CCL-β^{AS3}-FB LV and collected at day 14 of the erythroid differentiation culture assay were assessed for their HBβ^{AS3} mRNA expression by qRT-PCR and compared to expression from the endogenous adult HBB mRNA. The level of expression was similar in all the cultures; HBβ^{AS3} mRNA levels made up 55.2%±18.1% of the total HBB-like mRNA in erythroid cells from cultures of CB CD34⁺/38⁻ cells, compared to 45.4%±16.7% from cultures of CB CD34⁺ cells (p=0.59) (FIG. 17, panel B), which was similar to amounts reported in previous studies using the CCL-β^{AS3}-FB LV in healthy and SCD bone marrow CD34⁺ cells (Romero et al. (2013) J. Clin. Invest. 123: 3317-3330).

Assessment of LDL Receptor Expression after Prestimulation and Transduction

We evaluated whether differences in the recently identified cellular receptor for VSV-G-pseudotyped vectors (Finkelstein et al. (2013) Proc. Natl. Acad. Sci. USA, 110: 7306-7311), the human LDL receptor, were responsible for the more effective transduction of CD34⁺/CD38⁻ cells compared to CD34⁺ cells. Flow cytometry was used to analyze LDL receptor expression in fresh CB CD34⁺ and CD34⁺/CD38⁻ cells stained with APC anti-human LDL receptor antibodies and then again after 24 and 48 hours in culture in transduction medium with multiple cytokines (time points correlating to completion of prestimulation and transduction, respectively).

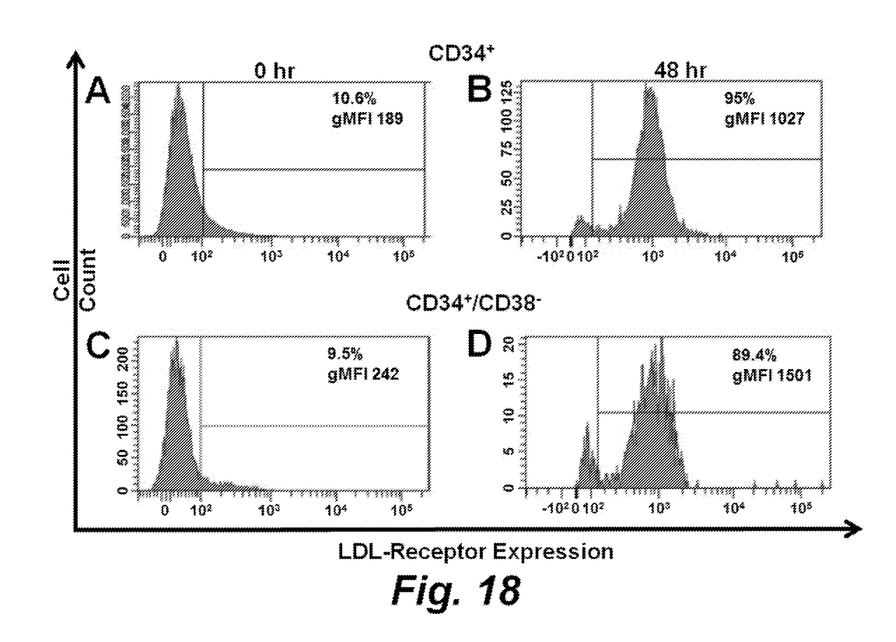

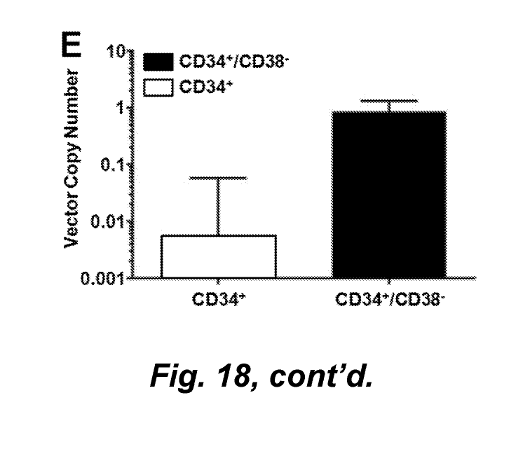

Fresh CD34⁺ cells (7.9%) expressed the LDL receptor compared to 4.8% of fresh CD34⁺/CD38⁻ cells (FIG. 18, panels A, D). At 24 hours, ˜91% of CD34⁺ cells were expressing the LDL receptor compared to 74.6% of CD34⁺/CD38⁻ cells (FIG. 18, panel B, 18, panel E). At 48 hours, LDL receptor was expressed on 99% of the CD34⁺ cells and on 90% of the CD34⁺/CD38⁻ cells (FIG. 18, panel C, F). The geometric mean fluorescence intensity of the LDL receptor was similar on the CD34⁺ and CD34⁺/CD38⁻ cells (1.6×10³-3×10³ and 1.5×10³-3.5×10³, respectively). Fresh CD34⁺ and CD34⁺/CD38⁻ cells had similarly low percentages expressing the LDL receptor, and it was induced equally on these cells by culture under conditions used for prestimulation and transduction. Thus, a difference in LDL receptor expression was not the basis for the better transduction of CD34⁺/CD38⁻ cells.

RD114 Retroviral Envelope Pseudotyped LV

To determine whether the improved transduction of the CD34⁺/CD38⁻ cells is specifically related to the VSV-G envelope, we produced a batch of the CCL-β^{AS3}-FB LV with an alternative pseudotype, using the RD114 retroviral envelope (Sandrin et al. (2002) Blood 100: 823-832; Bell et al. (2010) Exp. Biol. Med. 234: 1269-1276). CD34⁺/CD38⁻ cells transduced with the RD114-pseudotyped CCL-β^{AS3}-FB LV vector and then cultured for 14 days had a higher average VCN (0.86±0.46) compared to CD34⁺ cells transduced and analyzed under the same conditions (0.006±0.05, n=3) (FIG. 18, panel G). Therefore, the higher transduction of CD34⁺/CD38⁻ cells is not specific to the VSVG pseudotype.

Assessment of Engraftment Potential of CD34⁺/CD38⁻ Cells In Vivo

The prior studies compared transduction of CD34⁺/CD38⁻ cells isolated by FACS to unfractionated CD34⁺ cells. For subsequent studies, we compared the transduction and engraftment potential of CD34₊/CD38⁻ cells and CD34⁺/CD38⁺ cells, both isolated by FACS from the same populations of unfractionated CD34⁺. Each population was transduced with a lentiviral vector carrying different fluorescent reporter genes (CCLc-UBC-mCitrine-PRE-FB-2XUSE LV, CCLc-UBC-mCerulean-PRE-FB-2XUSE LV, and CCLc-UBC-mStrawberry-PRE-FB-2XUSE LV vectors). To ensure that the results were not influenced by the vector, the vector used to transduce each cell fraction was alternated for each study (n=3). Transduced CD34⁺, CD34⁺/CD38⁺, and CD34⁺/CD38⁻ cells were xenotransplanted into NSG mice at their appropriate physiologic proportions (99% CD34⁺/CD38⁺ cells+1% CD34⁺/CD38⁻ cells; or 100% CD34⁺ cells). Transduction conditions were the same as used for the in vitro analyses and the cells were transplanted immediately after 24 hours transduction. Each mouse received a total cell dose of 5×10⁵ cells consisting of (a) NT CD34⁺ cells (Mock), (b) transduced CD34⁺ cells (Control), or (c) a combination of transduced CD34⁺/CD38⁺ and transduced CD34⁺/CD38⁻ cells (Test), each mixed with irradiated (10,000 cGy) CD34⁻ cells as “fillers” (Table 5). The mice were euthanized 80-90 days after transplantation and their bone marrow was harvested to analyze engraftment of the human cells by flow cytometry and to measure VCN of engrafted cells. The percent engraftment was defined as the percentage of human CD45⁺ cells of the total CD45⁺ population (murine CD45⁺ plus human CD45⁺). BM from human engrafted mice was then further analyzed by flow cytometry for the percentage of the different transduced cell fractions present in the human engrafted cells, based on the fluorescent markers used and by ddPCR.

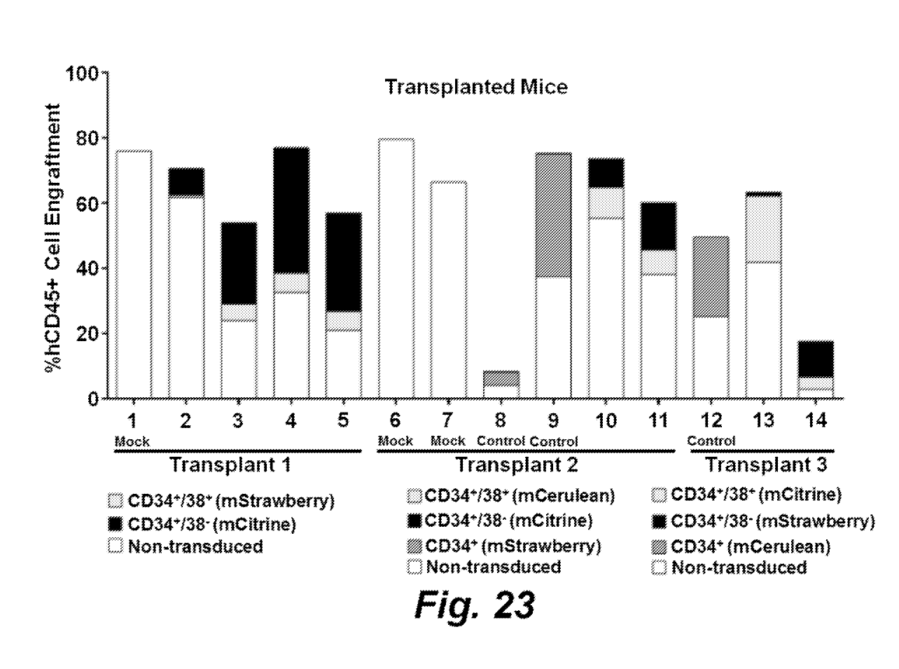

Three in vivo mouse transplants were conducted, each consisting of 6 mice, for a total of 18 mice transplanted (5 mock [NT CD34⁺ cells], 4 controls [transduced CD34⁺ cells], 9 test mice [mixture of transduced CD34⁺/38⁺ and transduced CD34⁺/38⁻ cells]) (Table 5). A portion of the transduced cells were grown in vitro for 2 weeks and assayed for VCN (Table 4). ddPCR primers and probes were designed to specifically detect each of the fluorescent marker genes (Table 3). Samples of transduced cells analyzed after 2 weeks in vitro cultures using the HIV-1 Psi region primers to measure all vectors showed that the CD34⁺/38⁻ cells consistently had a higher VCN (6.89±0.75) than the unfractionated CD34⁺ cells (VCN=2.19±0.47) or the CD34⁺/CD38⁺ cells (VCN=1.66±0.36) irrespective of the vector used for transduction (mCitrine, mStrawberry, mCerulean) (Table 4). Of the 18 total mice, 14 had successful engraftment of human CD45+ cells at the time of BM harvest (FIG. 23). Among the engrafted mice, mice #'s 1, 6, and 7 received only mock-transduced human CD34⁺ cells. Control mice #'s 8, 9, 12 received CD34⁺ cells transduced by a single vector. Mice #'s 2, 3, 4, 5, 10, 11, 13, and 14 received test mixtures of CD34⁺/CD38⁻ and CD34⁺/CD38⁺ cells (at 1:99 cell ratios) transduced with different vectors. Overall, there was a trend toward better engraftment with CD34⁺/CD38⁻ cells compared to unfractionated CD34⁺ cells (p=0.06).

TABLE 4
In vitro vector copy number of cells transplanted
into NOD.Cg-Prkd^{scid}II2rg^{tm1Wjil}/SzJ mice.

| Experimental Arm | In vitro vector copy number | | |
|---|---|---|---|
| Transplant # | 1 | 2 | 3 |
| Nontransduced CD34+ | 0 | 0 | 0 |
| Transduced CD34+ | — | 1.58 | 2.81 |
| Transduced CD34+/CD38+ | 1.48 | 0.40 | 2.66 |
| Transduced CD34+/CD38- | 1.94 | 7.59 | 11.50 |

TABLE 5
Experimental design of xenograft transplantation studies -- cell doses and fluorescent markers.

| | Transplant 1 | | Transplant 2 | | | Transplant 3 | |
|---|---|---|---|---|---|---|---|
| | Mock | Test | Mock | Control | Test | Control | Test |
| | Mouse #'s | | | | | | |
| Experimental Arm | 1 | 2, 3, 4, 5 | 6, 7 | 8, 9 | 10, 11 | 12 | 13, 14 |
| NT* CD34⁺ | 2 × 10⁵ | | 2 × 10⁵ | | | | |
| TD** CD34⁺ | | | | 2 × 10⁵ | | 2 × 10⁵ | |
| | | | | (mStrawberry) | | (mCerulean) | |
| TD CD34⁺/CD38⁺ | | 2 × 10⁵ | | | 2 × 10⁵ | | 2 × 10⁵ |
| | | (mStrawberry) | | | (mCerulean) | | (mCitrine) |
| TD CD34⁺/CD38⁻ | | 2 × 10³ | | | 2 × 10³ | | 2 × 10³ |
| | | (mCitrine) | | | (mCitrine) | | (mStrawberry) |
| NT CD34⁻ | 3 × 10⁵ | 3 × 10⁵ | 3 × 10⁵ | 3 × 10⁵ | 3 × 10⁵ | 3 × 10⁵ | 3 × 10⁵ |

*NT = not transduced;
**TD = transduced.

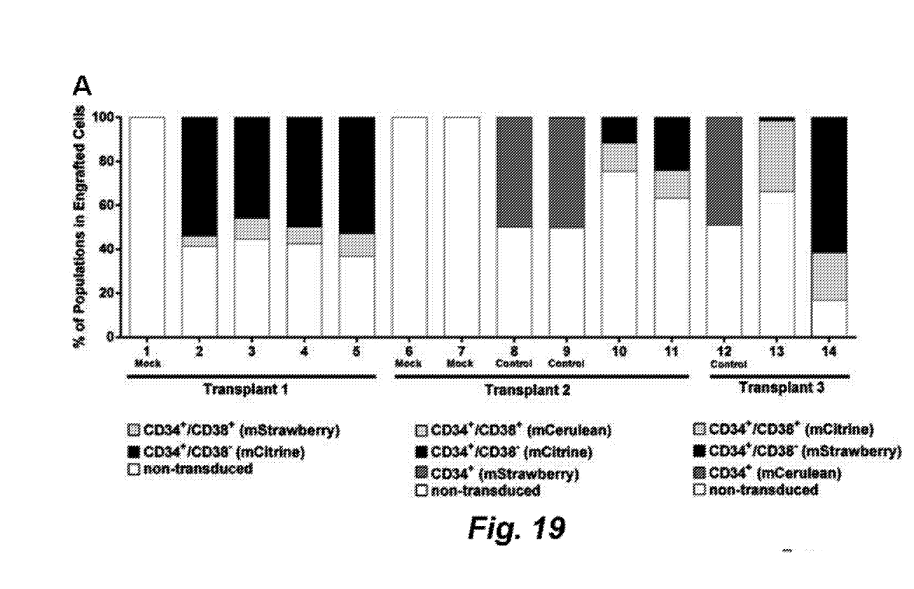

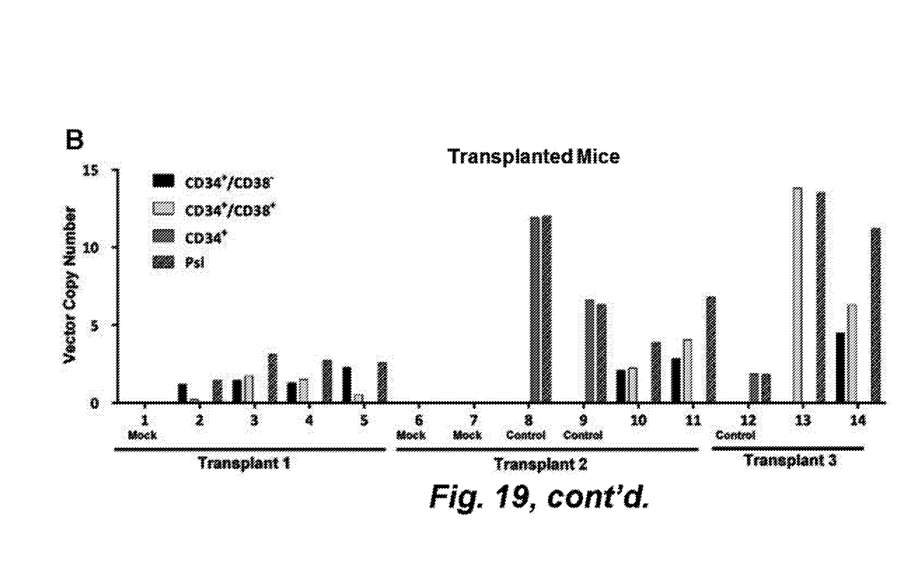

In the eight engrafted test mice, there was higher engraftment by the vector-labeled CD34⁺/CD38⁻ cells in six mice (#'s 2, 3, 4, 5, 11, and 14), equivalent engraftment of CD34⁺/CD38⁺ and CD34⁺/CD38⁻ cells in one mouse (#10), and higher CD34⁺/CD38⁺ engraftment in one mouse (#13) (FIG. 19, panel A, FIG. 23). Overall the gene marking levels by transplanted bulk CD34⁺ cells or the fractionated CD34⁺/CD38⁻ and the CD34⁺/CD38⁻ cells were not different (Table 6) and thus, transduced CD34⁺/CD38⁻ cells were approximately 100-times more potent for engraftment than the unfractionated CD34⁺ or CD34⁺/CD38⁺ cells.

TABLE 6
Digital droplet PCR (ddPCR) of bone marrow from NSG mice for each specific fluorescent marker gene used to mark individual
transplanted cell populations and for total vector proviruses (Psi), normalized to human genomes in each marrow sample.

| | | | | Fluorescent Gene Specific ddPCR | | | Total vector | |
|---|---|---|---|---|---|---|---|---|
| | Transplant | Fluorescent | | | | Sum | ddPCR | p |
| mouse # | Arm | Marker(s) | 34+ | 34+/38− | 34+/38+ | (38− + 38+) | (Psi) | value* |
| 8 | Bulk | mStrawberry | 11.9 | | | | 12 | |
| | CD34⁺ | | | | | | | |
| 9 | Bulk | mStrawberry | 6.61 | | | | 6.29 | |
| | CD34⁺ | | | | | | | |
| 12 | Bulk | mCerulean | 1.86 | | | | 1.81 | |
| | CD34⁺ | | | | | | | |
| Mean +/− S.D. | | | 6.79 +/− 5.02 | | | | 6.70 +/− 5.11 | 0.98 |
| 2 | 34⁺/38⁻ vs | mCitrine vs. | | 1.19 | 0.215 | 1.405 | 1.45 | |
| | C3⁺/38⁺ | mStrawberry | | | | | | |
| 3 | 34⁺/38⁻ vs | mCitrine vs. | | 1.44 | 1.74 | 3.18 | 3.12 | |
| | C3⁺/38⁺ | mStrawberry | | | | | | |
| 4 | 34⁺/38⁻ vs | mCitrine vs. | | 1.29 | 1.52 | 2.81 | 2.72 | |
| | C3⁺/38⁺ | mStrawberry | | | | | | |
| 5 | 34⁺/38⁻ vs | mCitrine vs. | | 2.27 | 0.54 | 2.81 | 2.58 | |
| | C3⁺/38⁺ | mStrawberry | | | | | | |
| 10 | 34⁺/38⁻ vs | mCitrine vs. | | 2.0-8 | 2.25 | 4.33 | 3.88 | |
| | C3⁺/38⁺ | mStrawberry | | | | | | |
| 11 | 34⁺/38⁻ vs | mCitrine vs. | | 2.84 | 4.06 | 6.9 | 6.79 | |
| | C3⁺/38⁺ | mStrawberry | | | | | | |
| 13 | 34⁺/38⁻ vs | mCitrine vs. | | 0 | 13.8 | 13.8 | 13.5 | |
| | C3⁺/38⁺ | mStrawberry | | | | | | |
| 14 | 34⁺/38⁻ vs | mCitrine vs. | | 4.49 | 6.28 | 10.777 | 11.2 | |
| | C3⁺/38⁺ | mStrawberry | | | | | | |
| Mean +/− S.D. | | | | | | 5.76 +/− 4.41 | 5.66 +/− 4.45 | 0.97 |
| Mean +/− S.D. | | | | 1.95 +/− 1.33 | 3.8 +/− 4.5 | | | 0.3 |

*By unpaired T test.

The two mice transplanted with bulk CD34⁺ cells transduced with a single vector had VCN of 12 and 6, with similar values measured using the HIV-1 Psi region primers or with the fluorescent marker-specific primers. The mice transplanted with a mixture of CD34⁺/CD38⁺ and CD34⁺/CD38⁻ cells showed similar levels of gene marking with the two vectors (3.60±0.26 and 2.17±0.15, respectively), and in each mouse, the sum of the VCN for the two individual vectors was similar to the total VCN measured using the Psi region primers (FIG. 19, panel B).

### Discussion

Stem cell gene therapy is advancing toward the clinic for multiple diseases including SCD. For it to be efficacious for SCD, transduction must be efficient with an adequate number of HSC transduced to express enough β^{AS3}-globin to change the pathophysiology of the disease. Clinical scale HSC transduction can be a challenging process made more difficult with large, complex gene cassettes being delivered and inserted, such as the β^{AS3}-globin gene. Although transduction of human CD34⁺ HSPC with the CCL-β^{AS3}-FB LV vector has been demonstrated (Romero et al. (2013) J. Clin. Invest. 123: 3317-3330), due to the suboptimal unconcentrated titers of the viral vector, high volumes of viral vector are required to attain the level of gene transfer to engrafting HSC to correct RBC disease manifestation of SCD. For these reasons, an alternate cell population that can be transduced using less viral vector to achieve comparable gene transduction efficiency with effective engraftment capabilities is appealing. To date, several studies have shown that CD34⁺/CD38⁻ cells can be transduced with LV vectors since these vectors are able to transduce cells that are not actively dividing, unlike c-retroviruses (Case et al. (1999) Proc. Natl. Acad. Sci. USA, 96: 2988-2993; Geronimi et al. (2003) Stem Cells, 21: 472-480; Guenechea et al. (2000) Mol. Ther. 1: 566-573). However, the capacity of CD34⁺/CD38⁻ cells to be transduced and engrafted for gene therapy has not been explored due to the absence of clinical-grade reagents and the challenges of large-scale GMP cell sorting.

We performed studies using human CB CD34⁺/CD38⁻ cells to assess the potential suitability of these cells for gene therapy of SCD while using less viral vector for transduction. Our studies have shown CD34⁺/CD38⁻ cells isolated from CB to be susceptible to transduction with lentiviral vectors, requiring markedly lower amounts of viral vector to achieve comparable or higher gene transfer compared to CD34⁺ cells. Importantly, clonal analysis of colony-forming progenitors indicated that a higher percentage were transduced when targeting the CD34⁺/CD38⁻ populations, compared to bulk CD34⁺ targets, rather than transducing a constant fraction but with higher vector copies. Ideally, clinical applications would lead to a similar higher percentage of transduced HSC for better efficacy, and limit the VCN per transduced cell for better safety.

Interestingly, in vivo transplant studies in NSG mice demonstrated that CD34⁺/CD38⁻ cells were approximately 100-fold more potent for engraftment than the counterpart CD34⁺/CD38⁻ cells, with essentially equivalent engraftment contributions. We were not able to obtain sufficient human cells from the bone marrow of the engrafted NSG mice to sort out the cells derived from the CD34⁺/CD38⁺ and CD34⁺/CD38⁻ cells based on their fluorescent marker, so that absolute VCNs per cell could not be directly measured to determine whether there was higher vector copies in the engrafted descendants of the CD34⁺/CD38⁻ cells, as was seen in vitro. Rather, ddPCR was performed with total marrow cells from engrafted NSG mice to quantify the specific fluorescent reporter genes used to mark the CD34⁺/CD38⁻ cells or the CD34⁺/CD38⁺ cells, normalized for the human cell content of the marrow and indicated similar contribution to hematopoiesis by the 1% CD34⁺/CD38⁻ cells as by the 99% CD34⁺/CD38⁻ that were transplanted. However, it is possible that the lack of higher average VCN of the vectors used to mark the CD34⁺/CD38⁻ cells in the marrow may indicate that high VCN led to cytotoxicity to transduced HSC and decreased contribution to engraftment.

Overall, these findings may have applications to any approach to gene therapy using HSC, in addition to the specific benefits for SCD gene therapy shown here. The use of CD34⁺/CD38⁻ cells in gene therapy would allow the use of lesser amounts of vector to transduce the target cells, but may still result in adequate engraftment, based on our observations in the xeno-transplant studies. These findings are consistent with other studies demonstrating the good engraftment capability of CD34⁺/CD38⁻ cells (Case et al. (1999) Proc. Natl. Acad. Sci. USA, 96: 2988-2993; Geronimi et al. (2003) Stem Cells, 21: 472-480; Guenechea et al. (2000) Mol. Ther. 1: 566-573; Haas et al. (2000) Mol. Ther. 2: 71-80).

Recent publications have described the LDL receptor as the major receptor for vesicular stomatitis virus (VSV) (Finkelstein et al. (2013) Proc. Natl. Acad. Sci. USA, 110: 7306-7311) that is most commonly used to pseudotype lentiviral vectors. If LDL receptor expression was higher in the CD34⁺/CD38⁻ cells, there is the possibility of more vector binding to the cell, being taken into the cell and eventually integrating into the genome, leading to the higher VCN seen. When expression of the LDL receptor was analyzed, in unfractionated CD34⁺ and CD34⁺/CD38⁻ cell populations, there were few cells expressing the LDL receptor at rest and relatively equivalent induction of expression 48 hours after stimulation with cytokines, corresponding to the time for transduction. It is therefore unlikely, that higher expression of the receptor for the VSV-G pseudotyped vector is the mechanism of increased transduction of CD34⁺/CD38⁻ cells.

When both cell populations were transduced with the CCL-β^{AS3}-FB LV vector pseudotyped with the RD-114 retroviral envelope protein, there was again higher transduction of the CD34⁺/CD38⁻ cells compared to CD34⁺ cells. These results reinforce the observation of increased susceptibility to transduction of CD34⁺/CD38⁻ cells despite use of a different envelope protein (Sandrin et al. (2002) Blood 100: 823-832; Bell et al. (2010) Exp. Biol. Med. 234: 1269-1276; Rasko et al. (1999) Proc. Natl. Acad. Sci. USA, 96: 2129-2134).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A recombinant retroviral vector, said vector comprising a human ubiquitin C (UBC) promoter and a multiple cloning site, wherein said UBC promoter is in a reverse orientation in said vector so that the direction of transcription from said promoter is oriented towards a 5′ long terminal repeat (LTR) of said vector and transcribes a nucleic acid inserted in said multiple cloning site.

2. A recombinant retroviral vector, said vector comprising a human ubiquitin C (UBC) promoter operably linked to a transgene wherein said promoter and said transgene are in a reverse orientation so that the direction of transcription of said transgene from said promoter is oriented towards a 5′ long terminal repeat (LTR) of said vector.

3. The vector according to any one of claims 1-2, wherein said promoter comprises or consists of a fragment from the human ubiquitin C gene UCSC human genome sequence version hg19 minus strand from about position 125398318 to about position 125399530.

4. The vector according to any one of claims 1-3, wherein an intron within said promoter is not lost during retroviral packaging.

5. The vector according to any one of claims 1-4, wherein said vector contains a polyadenylation signal in reverse orientation.

6. The vector of claim 5, wherein said polyadenylation signal (polyA) is inserted 3′ of said promoter which is 5′ of said promoter with respect to the entire vector sequence.

7. The vector according to any one of claims 5-6, wherein said polyadenylation signal is selected from the group consisting of a bovine growth hormone polyadenylation signal sequence, human growth hormone polyadenylation signal, a rabbit β-globin gene polyadenylation signal, a human herpes virus (HSV) polyadenylation signal, and a thymidine kinase (TK) gene polyadenylation signal.

8. The vector according to any one of claims 5-6, wherein said polyadenylation signal is a bovine growth hormone polyadenylation signal sequence or a human growth hormone polyadenylation signal.

9. The vector according to any one of claims 1-8, wherein said vector provides at least about a 2-fold increase in expression in transient transfected and stable-transduced cell lines as compared to the same vector with a UBC promoter in a non-reversed orientation.

10. The vector according to any one of claims 1-9, wherein said vector provides at least about a 4-fold increase in expression in transduced primary cells as compared to the same vector with a UBC promoter in a non-reversed orientation.

11. The vector according to any one of claims 1-10, wherein said retroviral vector is selected from group consisting of an HIV-1 lentiviral vector, an HIV-2 lentiviral vector, an alpharetroviral vector, an equine infectious anemia virus (EIAV) lentiviral vector, an MoMLV vector, an X-MLV vector, a P-MLV vector, a A-MLV vector, a GALV vector, an HEV-W vector, an SIV-1 vector, an FIV-1 vector, and an SERV-1-5 vector.

12. The vector of claim 11, wherein said retroviral vector is a lentiviral vector.

13. The vector of claim 12, wherein said retroviral vector is an HIV-1 based lentiviral vector.

14. The vector according to any one of claims 12-13, wherein said lentiviral vector is a TAT-independent and self-inactivating (SIN) lentiviral vector.

15. The vector according to any one of claims 1-14, wherein said vector is a bidirectional vector.

16. The vector according to any one of claims 1-15, further comprising an insulator in the 3′ LTR.

17. The vector of claim 16, wherein said insulator comprises FB (FII/BEAD-A), a 77 bp insulator element, which contains the minimal CTCF binding site enhancer-blocking components of the chicken β-globin 5′ DnaseI-hypersensitive site 4 (5′ HS4).

18. The vector according to any one of claims 1-17, wherein said vector comprises a w region vector genome packaging signal.

19. The vector according to any one of claims 1-18, wherein said vector comprises a Rev Responsive Element (RRE).

20. The vector according to any one of claims 1-19, wherein said vector comprises a central polypurine tract.

21. The vector according to any one of claims 1-20, wherein said vector comprises a post-translational regulatory element.

22. The vector of claim 21, wherein the posttranscriptional regulatory element is modified Woodchuck Post-transcriptional Regulatory Element (WPRE).

23. The vector according to any one of claims 1-22, wherein said vector is incapable of reconstituting a wild-type lentivirus through recombination.

24. The vector according to any one of claims 2-23, wherein said vector comprises a transgene operably linked to said UBC promoter wherein said transgene expresses a gene product for the treatment of a pathology selected from the group consisting of SCID, sickle cell disease, a liposomal storage disease, cystic fibrosis, muscular dystrophy, phenylketonuria, Parkinson's disease, and haemophilia.

25. The vector according to any one of claims 2-15, wherein said vector expresses one or more gene products selected from the group consisting of adenosine deaminase (ADA), IL-2 receptor gamma (IL-2Rγ), purine nucleoside phosphorylase (PNP) gene, Janus kinase-3 (JAK3), Artemis gene, anti-sickling human β-globin gene, Factor VIII, Factor IX, CFTR, full length or shortened dystrophin, ABCD1 gene, TH, AADC, and GCH1, Aspartylglucosaminidase, α-Galactosidase A, Palmitoyl Protein Thioesterase, Tripeptidyl Peptidase, Lysosomal Transmembrane Protein, Cysteine transporter, Acid ceramidase, Acid α-L-fucosidase, Protective protein/cathepsin A, Acid β-glucosidase, Acid β-galactosidase, Iduronate-2-sulfatase, α-L-Iduronidase, Galactocerebrosidase, Acid α-mannosidase, Acid β-mannosidase, Arylsulfatase B, Arylsulfatase A, N-Acetylgalactosamine-6-sulfate, Acid β-galactosidase, N-Acety lglucosamine-1-phosphotransferase, Acid sphingomyelinase (aSM), NPC-1, α-glucosidase, β-Hexosaminidase B, Heparan N-sulfatase, α-N-Acetylglucosaminidase, Acetyl-CoA: α-glucosaminide, N-Acetylglucosamine-6-sulfate, α-N-Acetylgalactosaminidase, α-N-Acetylgalactosaminidase, α-Neuramidase, β-Glucuronidase, β-Hexosaminidase A, Acid Lipase,

26. The vector of claim 24, wherein said transgene expresses adenosine deaminase (ADA) for the treatment of ADA-SCID.

27. The vector of claim 24, wherein said transgene expresses IL-2 receptor gamma (IL-2Rγ) gene/cDNA for the treatment of X-SCID.

28. The vector of claim 24, wherein said transgene expresses an anti-sickling human β-globin gene.

29. The vector of claim 28, wherein said anti-sickling human β-globin gene comprises about 2.3 kb of recombinant human β-globin gene including exons and introns under the control of the human β-globin gene 5′ promoter and the human β-globin 3′ enhancer.

30. The vector claim 29, wherein said β-globin gene comprises β-globin intron 2 with a 375 bp RsaI deletion from IVS2, and a composite human β-globin locus control region comprising HS2, HS3, and HS4.

31. A viral particle comprising a vector according to any one of claims 1-23.

32. A host cell transduced with a vector according to any one of claims 2-23.

33. The host cell of claim 32, wherein the cell is a stem cell.

34. The host cell of claim 33, wherein said cell is a stem cell derived from bone marrow.

35. The host cell of claim 33, wherein said cell is a stem cell that is not derived from an embryo or embryonic tissue.

36. The host cell of claim 32, wherein the cell is a 293T cell.

37. The host cell of claim 32, wherein, wherein the cell is a human hematopoietic progenitor cell.

38. The host cell of claim 37, wherein the human hematopoietic progenitor cell is a CD34⁺ cell.

39. The host cell of claim 37, wherein the human hematopoietic progenitor cell is a CD34⁺/CD38⁻ cell.

40. A composition for the treatment of a pathology shown in column A below, comprising a pharmaceutically acceptable carrier and a stem cell and/or progenitor cell transfected with a vector according to any one of claims 2-23, wherein said vector contains one or more transgenes for the treatment of said pathology as shown in column B below:

| A | B |
|---|---|
| Pathology | Transgene/gene product |
| ADA-SCID | adenosine deaminase (ADA) |
| X-SCID | IL-2 receptor gamma (IL-2Ry) |
| PNP-SCID | PNP gene |
| JAK3 | Janus kinase-3 (JAK3) |
| Artemis/DCLRE1C | Artemis gene |
| Sickle Cell Disease | anti-sickling human β-globin gene |
| Haemophilia A | Factor VIII |
| Haemophilia B | Factor IX |
| Cystic fibrosis | CFTR |
| Muscular Dystrophy | full length or shortened dystrophin |
| Adrenoleukodystrophy (ALD) | ABCD1 gene |
| Parkinson's Disease | TH, AADC, and GCH1 |
| Phenylketonuria | phenylalanine hydroxylase (PAH) |
| Aspartylglucosaminuria | Aspartylglucosaminidase |
| Fabry | α-Galactosidase A |
| Infantile Batten Disease | Palmitoyl Protein Thioesterase |
| Classic Late Infantile Batten Disease | Tripeptidyl Peptidase |
| Juvenile Batten Disease (CNL2) | Lysosomal Transmembrane Protein |
| Cystinosis | Cysteine transporter |
| Farber | Acid ceramidase |
| Fucosidosis | Acid α-L-fucosidase |
| Galactosidosialidosis | Protective protein/cathepsin A |
| Gaucher types 1, 2, and 3 | Acid β-glucosidase |
| GMl gangliosidosis | Acid β-galactosidase |
| Hunter | Iduronate-2-sulfatase |
| Hurler-Scheie | α-L-Iduronidase |
| Krabbe | Galactocerebrosidase. |
| α-Mannosidosis | Acid α-mannosidase. |
| β-Mannosidosis | Acid β-mannosidase |
| Maroteaux-Lamy | Arylsulfatase B |
| Metachromatic leukodystrophy | Arylsulfatase A |
| Morquio A | N-Acetylgalactosamine-6-sulfate |
| Morquio B | Acid β-galactosidase |
| Mucolipidosis II/III | N-Acety lglucosamine-1 -phospho- |
| | transferase |
| Niemann-PickA, B | Acid sphingomyelinase (aSM) |
| Niemann-Pick C | NPC-1 |
| Pompe Acid | α-glucosidase |
| Sandhoff | β-Hexosaminidase B |
| Sanfilippo A | Heparan N-sulfatase |
| Sanfilippo B | α-N-Acetylglucosaminidase |
| Sanfilippo C | Acetyl-CoA: α-glucosaminide |
| Sanfilippo D | N-Acetylglucosamine-6-sulfate |
| Schindler Disease | α-N-Acetylgalactosaminidase |
| Schindler-Kanzaki. | α-N-Acetylgalactosaminidase |
| Sialidosis | α-Neuramidase |
| Sly | β-Glucuronidase |
| Tay-Sachs | β-Hexosaminidase A |
| Wolman | Acid Lipase. |

41. The composition of claim 40, wherein said composition is for the treatment of ADA-SCID and said transgene expresses adenosine deaminase (ADA).

42. The composition of claim 40, wherein said composition is for the treatment of X-SCID and said transgene expresses IL-2 receptor gamma (IL-2Rγ).

43. The composition of claim 40, wherein said composition is for the treatment of sickle cell disease and said transgene expresses an anti-sickling human β-globin gene.

44. The composition of claim 43, wherein said anti-sickling human β-globin gene comprises about 2.3 kb of recombinant human β-globin gene including exons and introns under the control of the human β-globin gene 5′ promoter and the human β-globin 3′ enhancer.

45. The composition of claim 44, wherein said β-globin gene comprises β-globin intron 2 with a 375 bp RsaI deletion from IVS2, and a composite human β-globin locus control region comprising HS2, HS3, and HS4.

46. The composition according to any one of claims 40-45, wherein said host cell is a CD34⁺ cell.

47. The composition of claim 46, wherein said host cell is a CD34⁺/CD38⁻ cell.

48. A method for treating a subject for a pathology shown in column A below, comprising introducing into said subject progenitor or stem cells transfected with a vector according to any one of claims 2-23, wherein said vector contains one or more transgenes for the treatment of said pathology as shown in column B below:

| A | B |
|---|---|
| Pathology | Transgene/gene product |
| ADA-SCID | adenosine deaminase (ADA) |
| X-SCID | IL-2 receptor gamma (IL-2Rγ) |
| PNP-SCID | PNP gene |
| JAK3 | Janus kinase-3 (JAK3) |
| Artemis/DCLRE1C | Artemis gene |
| Sickle Cell Disease | anti-sickling human β-globin gene |
| Haemophilia A | Factor VIII |
| Haemophilia B | Factor IX |
| Cystic fibrosis | CFTR |
| Muscular Dystrophy | full length or shortened dystrophin |
| Adrenoleukodystrophy (ALD) | ABCD1 gene |
| Parkinson's Disease | TH, AADC, and GCH1 |
| Phenylketonuria | phenylalanine hydroxylase (PAH) |
| Aspartylglucosaminuria | Aspartylglucosaminidase |
| Fabry | α-Galactosidase A |
| Infantile Batten Disease | Palmitoyl Protein Thioesterase |
| Classic Late Infantile Batten Disease | Tripeptidyl Peptidase |
| Juvenile Batten Disease (CNL2) | Lysosomal Transmembrane Protein |
| Cystinosis | Cysteine transporter |
| Farber | Acid ceramidase |
| Fucosidosis | Acid α-L-fucosidase |
| Galactosidosialidosis | Protective protein/cathepsin A |
| Gaucher types 1, 2, and 3 | Acid β-glucosidase |
| GMl gangliosidosis | Acid β-galactosidase |
| Hunter | Iduronate-2-sulfatase |
| Hurler-Scheie | α-L-Iduronidase |
| Krabbe | Galactocerebrosidase. |
| α-Mannosidosis | Acid α-mannosidase. |
| β-Mannosidosis | Acid β-mannosidase |
| Maroteaux-Lamy | Arylsulfatase B |
| Metachromatic leukodystrophy | Arylsulfatase A |
| Morquio A | N-Acetylgalactosamine-6-sulfate |
| Morquio B | Acid β-galactosidase |
| Mucolipidosis II/III | N-Acety lglucosamine-1 -phospho- |
| | transferase |
| Niemann-PickA, B | Acid sphingomyelinase (aSM) |
| Niemann-Pick C | NPC-1 |
| Pompe Acid | α-glucosidase |
| Sandhoff | β-Hexosaminidase B |
| Sanfilippo A | Heparan N-sulfatase |
| Sanfilippo B | α-N-Acetylglucosaminidase |
| Sanfilippo C | Acetyl-CoA: α-glucosaminide |
| Sanfilippo D | N-Acetylglucosamine-6-sulfate |
| Schindler Disease | α-N-Acetylgalactosaminidase |
| Schindler-Kanzaki. | α-N-Acetylgalactosaminidase |
| Sialidosis | α-Neuramidase |
| Sly | β-Glucuronidase |
| Tay-Sachs | β-Hexosaminidase A |
| Wolman | Acid Lipase. |

49. The method of claim 48, wherein said method is for the treatment of ADA-SCID and said transgene expresses adenosine deaminase (ADA).

50. The method of claim 48, wherein said method is for the treatment of X-SCID and said transgene expresses IL-2 receptor gamma (IL-2Rγ).

51. The method of claim 48, wherein said method is for the treatment of sickle cell disease and said transgene expresses an anti-sickling human β-globin gene.

52. The method of claim 51, wherein said anti-sickling human β-globin gene comprises about 2.3 kb of recombinant human β-globin gene including exons and introns under the control of the human β-globin gene 5′ promoter and the human β-globin 3′ enhancer.

53. The method of claim 52, wherein said β-globin gene comprises β-globin intron 2 with a 375 bp RsaI deletion from IVS2, and a composite human β-globin locus control region comprising HS2, HS3, and HS4.

54. The method according to any one of claims 48-53, wherein said introducing comprises
transducing a stem cell and/or progenitor cell from said subject with said vector; and
transplanting said transduced cell or cells derived therefrom into said subject where said cells or derivatives therefrom express said transgene.

55. The method according to any one of claims 48-54, wherein, wherein the cell is a progenitor cell.

56. The method according to any one of claims 48-54, wherein the cell is a stem cell.

57. The method according to any one of claims 48-56, wherein said cell is a derived from bone marrow.

58. The method according to any one of claims 48-57, wherein said cell is a CD34⁺ cell.

59. The method of claim 58, wherein said cell is a CD34⁺/CD38⁻ cell.

60. The method according to any one of claims 48-59, wherein said cell is derived from said subject.

61. A population of cells that provide improved transduction with a recombinant lentivirus, said population of cells being enriched for CD34⁺/CD38⁻ cells.

62. The population of cells of claim 61, wherein said CD34+/CD38− cells are derived from blood or bone marrow.

63. The population of according to any one of claims 61-62, wherein said CD34+/CD38− cells are transfected with a retroviral vector containing a transgene.

64. The population of cells of claim 63, wherein said CD34+/CD38− cells are transduced with a retroviral vector selected from group consisting of an HIV-1 lentiviral vector, an HIV-2 lentiviral vector, an alpharetroviral vector, an equine infectious anemia virus (EIAV) lentiviral vector, an MoMLV vector, an X-MLV vector, a P-MLV vector, a A-MLV vector, a GALV vector, an HEV-W vector, an SIV-1 vector, an FIV-1 vector, and an SERV-1-5 vector.

65. The population of cells of claim 63, wherein said CD34+/CD38− cells are transduced with a lentiviral vector.

66. The population of cells of claim 65, wherein said CD34+/CD38− cells are transduced with a TAT-independent and self-inactivating (SIN) lentiviral vector.

67. The population of cells according to any one of claims 63-66, wherein said transgene is a transgene to treat a pathology listed in Table 1.

68. The population of cells according to any one of claims 63-66, wherein said transgene encodes ADA, IL-2γR, or an antisickling gene.

69. The population of cells of claim 63, wherein said cells are transfected with a CCL-βAS3-FB LV.

70. A method of improving transduction of stem cells or progenitor cells comprising providing for said transduction a population of stem cells or progenitor cells that are enriched for CD34+/CD38− cells.
